(19) United States
(12) Patent Application Publication
(10) Pub. No.: US 2006/0182726 A1
(43) Pub. Date: Aug. 17, 2006

(54) Immunomodulating compositions, processes for their production and uses therefor

(75) Inventors: Ranjeny Thomas, Queensland (AU); Brendan John O'Sullivan, Queensland (AU)

Correspondence Address: HELLER EHRMAN WHITE & MCAULIFFE LLP, 1717 RHODE ISLAND AVE, NW, WASHINGTON, DC, 20036-3001, US

(73) Assignee: The University of Queensland, Queensland (AU)

(21) Appl. No.: 10/524,539

(22) PCT Filed: Aug. 12, 2003
(86) PCT No.: PCT/AU03/01021

§ 371 Date: Sep. 28, 2005

Related U.S. Application Data
(60) Provisional application No. 60/403,131, filed on Aug. 12, 2002.

(30) Foreign Application Priority Data
Dec. 4, 2002 (AU) 2002953094

(51) Int. Cl. A61K 48/00; A61K 39/00; C12N 5/08

(52) U.S. Cl. 424/93.21; 435/372; 424/184.1

(57) ABSTRACT

The present invention discloses compositions and methods for antigen-specific suppression of immune responses, including primed immune responses. In particular, the invention discloses antigen-presenting cells, especially dendritic cells, whose level and or functional activity of CD40, or its equivalent, is impaired, abrogated or otherwise reduced, and their use for treating and/or preventing unwanted or deleterious immune responses including those that manifest in autoimmune disease, allergy and transplant rejection.

### FIELD OF THE INVENTION

THIS INVENTION relates generally to modulation of immune responses. More particularly, the present invention relates to compositions and methods for antigen-specific suppression of immune responses, including primed immune responses. Even more particularly, the invention is directed to the use of antigen-presenting cells, especially dendritic cells, whose level and or functional activity of CD40, or its equivalent, is impaired, abrogated or otherwise reduced, for treating and/or preventing unwanted or deleterious immune responses including those that manifest in autoimmune disease, allergy and transplant rejection.

Bibliographic details of various publications referred to by author in this specification are collected at the end of the description.

### BACKGROUND OF THE INVENTION

Antigen-specific suppression of a previously primed immune response is a major challenge for immunotherapy of autoimmune disease. Targeting the antigen-presenting cell (APC)-T cell interaction as a therapeutic approach to human autoimmune disease lags behind the blockade of pro-inflammatory and tissue destructive cytokines. Blockade of products of the innate immune system, including TNF-α and IL-1β, produces dramatic anti-destructive clinical effects in the autoimmune disease rheumatoid arthritis (RA) (reviewed by (Feldmann and Maini, 2001)). However, this approach is non antigen-specific and is reversible in the absence of treatment. Novel approaches include reinstitution of control on self-antigen presentation, derived from regulatory T cells (Treg). Suppression of immune effector cells by a variety of described Treg is a key mechanism for peripheral tolerance (Maloy and Powrie, 2001; Roncarolo and Levings, 2000). However, for control of pre-existing autoimmune or other immune responses, it will be important to understand the major determinants of regulation of antigen presentation by APC.

The complex interactions resulting in the generation of T cell-mediated immune responses are dependent on antigen presentation, cognate interactions between T cells and antigen presenting cells (APCs) including dendritic cells (DCs), and the concomitant production of soluble and membrane costimulatory molecules by APCs and T cells (reviewed by (Banchereau et al., 2000)). Various studies implicate DCs in the establishment and maintenance of self tolerance—both centrally and in the periphery (Ardavin et al., 1993; Mellman and Steinman, 2001). The molecular mechanisms controlling DC function in tolerance and immunity are poorly defined. However, B cells deprived of signalling through CD40 have been shown to induce T cell tolerance (Buhlmann et al., 1995; Hollander et al., 1996). A role for DCs in the induction of peripheral tolerance through Treg has been supported by several recent studies. The ability of myeloid DC to induce immunity or tolerance appears to be linked to its maturation state (Dhodapkar et al., 2001; Jonuleit et al., 2000; Lutz et al., 2000; Mehling et al, 2000; Roncarolo et al., 2001). Immature DCs generated from murine BM induced T cell unresponsiveness in vitro and prolonged cardiac allograft survival in a preventive model (Lutz et al., 2000). Immature myeloid DCs induced CD4⁺ Treg in vitro and CD8⁺ Treg in Vivo which each produced high levels of IL-10 and low levels of IFN-γ, but no IL-4 (Dhodapkar et al., 2001; Jonuleit et al., 2000). Various drugs and cytokines, and inhibitors of NT-κB have been shown to inhibit myeloid DC maturation (de Jong et al., 1999; Griffin et al., 2001; Hackstein et al., 2001; Lee et al., 1999; Mehling et al., 2000; Steinbrink et al., 1997; Yoshimura et al., 2001). DCs generated in the presence of these agents altered T cell function in vitro and in vivo, including promotion of allograft survival (Giannoukakis et al., 2000; Griffin et al., 2001). Despite this, suppression of previously primed CD4⁺ T cell responses by DCs in vivo has not been demonstrated. This is important for therapy of pre-existing autoimmune disease as CD4⁺ effector T cells mediate the perpetuation of tissue damage in autoimmune disease through their interaction with monocytes, B cells and local DCs (Feldmann, 2001; MacDonald et al., 1997; Sakata et al., 1996).

The present invention is predicated in part on the unexpected discovery that inhibition of NF-κB activity, especially RelB inhibition, in precursors of APCs leads to the production of APCs with reduced or abrogated CD40 expression, which can not only prevent priming of immunity but which can also suppress a previously primed immune response. Surprisingly, these APCs induce the differentiation of CD4⁺ regulatory T cells that can transfer tolerance to antigen-primed recipients in an IL-10 dependent manner. The foregoing discovery has been reduced to practice in the form of immunomodulating compositions and methods of treatment or prophylaxis, as described hereinafter.

### SUMMARY OF THE INVENTION

Accordingly, in one aspect of the present invention, there is provided an isolated antigen-presenting cell for modulating an immune response, which is characterised by producing CD40, or its equivalent, at a level and/or functional activity which is lower than that produced by an activated dendritic cell. In some embodiments, the antigen-presenting cell is other than a B lymphocyte. Suitably, the antigen-presenting cell produces CD40, or its equivalent, at a level and/or functional activity that is less than about 1% of that produced by an activated dendritic cell. In a preferred embodiment of this type, the antigen-presenting cell produces CD40, or its equivalent, at a level and/or functional activity that is lower than that produced by an immature dendritic cell. The antigen-presenting cell is preferably selected from monocytes, macrophages, B lymphocytes and their precursors as well as other cells of myeloid lineage, dendritic cells or Langerhans cells but is more preferably selected from dendritic cells and macrophages.

Suitably, the antigen-presenting cell is further characterised by producing NF-κB or component thereof, especially RelB, at a level and/or functional activity which is lower than that produced by a mature or activated dendritic cell. In a preferred embodiment of this type, the antigen-presenting cell produces NF-κB or component thereof, especially RelB, at a level and/or functional activity that is lower than that produced by an immature dendritic cell.

Preferably, the antigen-presenting cell is further characterised by producing an immunostimulatory molecule, especially CD86 or its equivalent. Desirably, the immunostimulatory molecule is produced at a level and/or functional activity which is at least about 10% of, but preferably about the same as, that produced by an activated dendritic cell.

In one embodiment, the antigen-presenting cell is produced by a process comprising contacting a precursor of the antigen-presenting cell with an NF-κB inhibitor for a time and under conditions sufficient to differentiate an antigen-presenting cell from the precursor and to inhibit or otherwise reduce the level and/or functional activity of NF-κB in said cell. The precursor is preferably derived from monocytes or bone marrow. In a preferred embodiment, the NF-κB inhibitor inhibits nuclear translocation of NF-κB or component thereof, especially RelB.

In another embodiment, the antigen-presenting cell is produced by a process comprising contacting an antigen-presenting cell, or its precursor, with an inhibitor of CD40, or its equivalent, for a time and under conditions sufficient to produce a modified antigen-presenting cell that produces CD40, or its equivalent, at a reduced or abrogated level and/or functional activity relative to that of said antigen-presenting cell or its precursor. Optionally, the process is further characterised by contacting the antigen-presenting cell, or its precursor, or the modified antigen-presenting cell, with an agent that increases the level and/or functional activity of an immunostimulatory molecule, especially of CD86 or its equivalent, for a time and under conditions sufficient to enhance or otherwise elevate the level and/or functional activity of the immunostimulatory molecule in the antigen-presenting cell, or its precursor, or the modified antigen-presenting cell.

Another aspect of the present invention contemplates a method of producing antigen-presenting cells for modulating an immune response to a target antigen, comprising contacting an antigen-presenting cell as broadly described above with an antigen that corresponds to the target antigen, or with a polynucleotide from which the antigen is expressible, for a time and under conditions sufficient for the antigen or a processed form thereof to be presented by the antigen-presenting cell. Typically, the antigen presentation is restricted by major histocompatability (MHC) molecules.

In a related aspect, the invention encompasses an antigen-specific antigen-presenting cell for modulating an immune response to a target antigen, which is produced by contacting an antigen-presenting cell as broadly described above with an antigen that corresponds to the target antigen, or with a polynucleotide from which the antigen is expressible, for a time and under conditions sufficient for the antigen or a processed form thereof to be presented by the antigen-presenting cell.

In another related aspect, the invention provides a method of producing antigen-presenting cells for modulating an immune response to a target antigen, comprising contacting a precursor of the antigen-presenting cell with an NF-κB inhibitor and with an antigen that corresponds to the target antigen, or with a polynucleotide from which the antigen is expressible, for a time and under conditions sufficient to differentiate an antigen-presenting cell from the precursor and to inhibit or otherwise reduce the level and/or functional activity of NF-κB in said cell, wherein the antigen or a processed form thereof is presented by the antigen-presenting cell so produced.

The antigen-specific antigen-presenting cell as broadly described above is also useful for producing antigen-specific regulatory T lymphocytes for suppression of an immune response to that antigen. Accordingly, in yet another aspect, the invention provides a method for producing T lymphocytes that exhibit anergy for a target antigen, comprising contacting a population of T lymphocytes, or their precursors, with an antigen-specific antigen-presenting cell as broadly described above for a time and under conditions sufficient to produce said anergic T lymphocytes.

The antigen-specific antigen-presenting cell and T lymphocytes as broadly defined above are especially useful for inducing a tolerogenic response including the induction of an anergic response, or the suppression of a future or existing immune response, to a specified antigen or group of antigens. For example, the antigen-specific immune response includes, but is not limited to, a response mediated by T lymphocytes such as cytotoxic T lymphocytes (CTLs) and T helper lymphocytes. The antigen-specificity may be to an antigen selected from a protein antigen, a particulate antigen, an alloantigen, an autoantigen, an allergen, a bacterial antigen, a viral antigen or a parasitic antigen or immune complex.

Accordingly, in still another aspect, the invention embraces a method for modulating the immune response to an antigen, comprising administering to a patient in need of such treatment one or both of an antigen-specific antigen-presenting cell as broadly described above and an anergic T lymphocyte as broadly described above for a time and under conditions sufficient to modulate said immune response.

In a related aspect, the invention extends to the use of the aforesaid antigen-specific antigen-presenting cell and/or said anergic T lymphocyte as broadly described above in methods for inducing an anergic response, or for treating or preventing an allergy or an autoimmune disease, or for treating or preventing transplant rejection in a patient, by administering to a patient in need thereof an effective amount of one or both of an antigen-specific antigen-presenting cell as broadly described above and an anergic T lymphocyte as broadly described.

In another related aspect, the invention encompasses a method for treatment and/or prophylaxis of a disease or condition whose symptoms or aetiology are associated with the presence of an immune response, comprising administering to a patient in need of such treatment or prophylaxis an effective amount of one or both of an antigen-specific antigen-presenting cell as broadly described above and an anergic T lymphocyte as broadly described.

In still yet another aspect, the invention contemplates the use of an antigen-presenting cell or an antigen-specific antigen-presenting cell as broadly described above or an anergic T lymphocyte as broadly described above in the preparation of a medicament for the modulation of an immune.

The invention also encompasses the use of an antigen-presenting cell or an antigen-specific antigen-presenting cell as broadly described above or an anergic T lymphocyte as broadly described in the study and modulation of immune responses.

### BRIEF DESCRIPTION OF THE DRAWINGS

FIG. 1 is a graphical representation showing suppression of primed immune responses by RelB deficient bone marrow-derived dendritic cells (BMDCs). BMDCs were generated from wild-type or RelB^{-/-} mice and cell surface markers were analysed by flow cytometry (a). Wild type mice were injected with BMDCs or methylated bovine serum albumin (mBSA) in complete Freund's adjuvant (CFA) as shown, and draining lymph node (DLN) mBSA-specific T cell proliferation was examined 7 days later (b). Wild type mice were injected with BMDCs or saline as shown, 7 days before or after immunization with keyhole limpet haemocyanin (KLH) in CFA (c). DLN KLH-specific T cell proliferation, and ear KLH-specific delayed-type hypersensitivity (DTH) responses are shown. Mean±SEM cpm from groups of 5 mice tested individually are shown. Results are representative of two separate experiments. * p<0.05: ** p<0.01 and *** p<0.001.

FIG. 2 is a diagrammatic and graphical representation showing that dendritic (DC) differentiation in the presence of an inhibitor of NF-κB translocation inhibits CD40 expression and APC function. BMDCs were generated in the presence or absence of BAY 11-7082. Nuclear and cytoplasmic extracts were immunoblotted for NF-κB subunits as shown (a). Cell surface marker expression was analysed by flow cytometry (b). Naïve C57BL/6 mice were injected subcutaneously (s.c.) as shown. DLN T cell proliferation in vitro in response to exogenous mBSA is displayed as mean±SEMΔCPM of triplicates of 5 mice assayed individually (c). Results are representative of three separate experiments.

FIG. 3 is a graphical representation showing suppression of primed immune responses by inhibition of RelB function of DCs. Mice were injected s.c. with BMDCs as shown, 7 d before (a) or after (b) priming with mBSA in CFA. 5 days later, mice were individually tested for DLN antigen-specific T cell proliferative, serum antibody and ear DTH responses. 7 days after s.c. or intravenous (i.v.) immunisation with varying doses of mBSA-pulsed BAY-treated BMDCs or no DCs, mice were injected with mBSA in CFA (c) or KLH in CFA (d). DTH responses to mBSA or KLH were measured 5 days later. (e): 5×10⁵ KLH-pulsed H-2^{b} wild type or MHC class II^{-/-} BMDCs generated in the presence or absence of BAY were administered to wild type mice 7 days after immunization with KLH in CFA. KLH-specific immunity was tested 5 days after DC administration. Results are displayed as the mean±SEM for each group (n=5), tested separately, and are representative of three separate experiments. NS not significant, * p<0.01, ** p<0.001.

FIG. 4 is a graphical representation showing suppression of primed responses by DCs correlates with RelB nuclear binding activity. BMDCs were generated in either GM-CSF and IL-4 (control), GM-CSF alone (immature) or GM-CSF and IL-4 in the presence of BAY, and cell surface markers were analysed (a). Mice were primed with KLH in CFA and 7 days later injected with DCs or saline as shown. KLH-specific T cell proliferative responses were measured after 5 days (b). Nuclear extracts from DCs were bound to ELISA plates coated with NF-κB consensus oligonucleotides, and detected with either anti-RelB or anti-p50 (c).

FIG. 5 is a graphical representation showing CD40 deficiency is sufficient to confer suppression of immunity by DCs. BMDCs were generated from CD40^{-/-} H-2^{d} mice in the presence or absence or BAY and cell surface markers were analysed by flow cytometry (a). Mice were injected with BMDCs or saline as shown, 7 days after immunisation with KLH in CFA. DLN KLH-specific T cell proliferation (b), and ear KLH-specific DTH responses (c) are shown. Mean±SEM cpm from groups of 5 mice tested individually are shown.

FIG. 6 is a graphical representation showing antigen-specific tolerance is “infectious”. Mice were injected with 5×10⁵ BMDCs as shown. Spleens were collected after 7 days and 5×10⁵ CD4⁺CD3⁺ or CD4⁻CD3⁺ cells sorted from nylon wool purified preparations were injected i.v. into non-irradiated KLH or ovalbumin (OVA)-primed syngeneic mice. Mean±SEMΔCPM KLH or OVA-specific T cell proliferative responses in DLN (a) or DTH responses (b) measured 7 days later from groups of 3 mice tested individually are shown. (c): 5×10⁵ DCs generated from BM 4 of H-2^{b} wild type mice in the presence or absence of BAY were pulsed with KLH and administered s.c. to naïve IL-10^{+/+} (white bars) or L-10^{-/-} (black bars) H-2^{b} mice. CD4⁺ T cells were magnetically sorted by negative selection from recipient spleens 7 days later and 2.5×10⁵ cells were transferred to wild type mice primed 7 days previously with KLH in CFA. Data are representative of 2 separate experiments. ***p<0.0001, **p<0.001.

FIG. 7 is a graphical representation showing that nuclear extracts from BAY-treated monocyte-derived DC (MDDC) lack RelB-DNA binding and are unresponsive to LPS. MDDC were generated for 48 h from PB monocytes in the presence of GM-CSF and IL-4 in the presence (BAY DC) or absence (control DC) of 8 μM BAY, then stimulated with 1 μg/mL LPS for 24 hr. Nuclear extracts were prepared and analysed for NFkB DNA binding by ELISA.

FIG. 8 is a graphical representation showing that modified human DC lack CD40 compared to immature DC. MDDC were generated for 48 h from PB monocytes in the presence of GM-CSF and IL-4 in the presence (modified DC) or absence (immature DC) of BAY, then stained for various cell surface markers and analysed by flow cytometry.

FIG. 9 is a graphical representation showing that modified human DC are unresponsive to CD40L. MDDC were generated for 48 h from PB monocytes in the presence of GM-CSF and IL-4 in the presence (modified DC) or absence (immature DC) of BAY, washed then incubated with or without 50 ng/ml soluble CD40L. Cells were then stained for CD86 and HLA-DR and analysed by flow cytometry.

FIG. 10 is a graphical representation showing that T cells fail to proliferate when stimulated by modified DC. MDDC were generated for 72 h from PB monocytes in the presence of GM-CSF and IL-4 in the presence (modified DC) or absence (immature DC) of BAY, or 48 h old immature DC were incubated for the final 24 h with 500 ng/ml LPS (mature DC), washed, then incubated with purified resting allogeneic T cells (FIG. 10A), or with purified resting autologous T cells in the presence or absence of tetanus toxoid or hepatitis B surface antigen particles (FIG. 10B). T cell proliferation was assessed in each case by [³H] thymidine incorporation after a total of 5 days.

FIG. 11 is a graphical representation showing that T cells remain viable when stimulated by modified DC. MDDC were generated for 72 h from PB monocytes in the presence of GM-CSF and IL-4 in the presence (modified DC) or absence (immature DC) of BAY, or 48 h old immature DC were incubated for the final 24 h with 500 ng/ml LPS (mature DC), washed, then incubated with purified resting allogeneic T cells for a total of 10 days. Control T cells were incubated in the absence of DC. Viability was measured flow cytometrically by the % cells unstained after propidium iodide addition.

FIG. 12 is a graphical representation showing that modified DC do not induce IFNγ production by T cells. MDDC were generated for 48 h from PB monocytes in the presence of GM-CSF and IL-4 in the presence (modified DC) or absence (immature DC) of BAY, washed, then incubated with purified resting allogeneic T cells. Interferon-γ was assayed 3 days later in the culture supernatants by ELISA.

FIG. 13 is a graphical representation showing that the level of CD40 expression by DC correlates with the T cell proliferative response in MLR. MDDC were generated for 48 h from PB monocytes in the presence of GM-CSF and IL-4 in the presence of varying concentrations of BAY, and the expression of CD40 was determined by flow cytometry. DC from each culture were also washed then incubated with purified resting allogeneic T cells. T cell proliferation was assessed by [³H] thymidine incorporation after a total of 5 days. % CD40⁺ DC and [³H] thymidine uptake in response to stimulation of 1×10⁵ allogeneic T cells by 5×10³ DC is shown for each DC culture. Regression line with 95% confidence interval curves is shown. Individual data points show triplicate means±SEM.

FIG. 14 is a graphical representation showing that modified DC pulsed with arthritogenic antigen suppress antigen-induced arthritis in mice after clinical disease onset. Mono-articular antigen-induced arthritis (AIA) was induced in male C57/B16 mice as previously described (van den Berg W et al., 1982). Briefly, on day −8, mice were primed subcutaneously in each axilla with 100 mg mBSA, complete Freunds adjuvant and 2% Tween 80, and 400 ng pertussis toxin were injected intra-peritoneally. On day 0, 60 mg mBSA in 10 ml saline were injected into one knee and 10 ml saline into the contra-lateral knee, under anaesthesia. Murine DC were generated for 7 days from bone marrow precursors in serum free medium, in the presence of 10 ng/ml GM-CSF, 10 ng/ml IL-4 and 2 μM BAY11-7082, pulsed with mBSA, then washed. Mice were injected s.c. into the tail base with 0.5×10⁶ DC either 2, 4 or 6 days after the knee joint injections, or left untreated (AIA control). Joints were graded on alternate days for severity using a semi-quantitative scale from 0 (normal) to 5 (severe) based on the degree of swelling, measured by callipers under anaesthesia (graph shows mean±SEM). The mean swelling score of the control joints was 0 (data not shown).

FIG. 15 is a graphical representation showing that suppression of ALA by modified DC is antigen-specific. ALA was induced as described for FIG. 14. Murine DC were generated for 7 days from bone marrow precursors in serum free medium, in the presence of 10 ng/ml GM-CSF, 10 ng/ml IL-4 and in the presence or absence of 2 μM BAY11-7082, pulsed with either mBSA or KLH, then washed. Mice were injected s.c. into the tail base with 0.5×10⁶ DC either 2, 4 or 6 days after the knee joint injections, or left untreated (AIA control). Joints were graded on day 10 using a semi-quantitative scale from 0 (normal) to 5 (severe) based on the degree of swelling. The mean swelling of the control joints was 0 (data not shown).

FIG. 16 is a graphical representation showing treatment of full blown inflammatory arthritis with NFkB⁻ DC is equivalent to TNFα neutralization. Mono-articular antigen-induced arthritis (ALA) was induced in male C57/B16 mice as described for FIG. 14. Murine DC were generated for 7 days from bone marrow precursors in serum free medium, in the presence of 10 ng/ml GM-CSF, 10 ng/ml IL-4 and 7 μM BAY11-7082, pulsed with mBSA, then washed. Mice were injected s.c. into the tail base with 0.5×10⁶ DC 6 days after the knee joint injections, or i.p. with 100 μg/mL anti-TNFα, both DC and anti-TNFα, or left untreated (ALA control). 14 days after knee joint injection of mBSA, the same joints were challenged with 100 U IL-1β. 2 days later mice were either treated with 0.5×10⁶ DC or irrelevant antibody. Joints were graded on alternate days for severity using a semi-quantitative scale from 0 (normal) to 5 (severe) based on the degree of swelling, measured by callipers under anesthesia (graph shows mean±SEM). The mean swelling of the control joints was 0 (data not shown).

FIG. 17 is a graphical representation showing alteration in antigen-specific antibody isotype in arthritic mice treated with NFkB⁻ DC. Mono-articular antigen-induced arthritis (ALA) was induced in male C57/B16 mice as described for FIG. 16. Murine DC were generated for 7 days from bone marrow precursors in serum free medium, in the presence of 10 ng/mL GM-CSF, 10 ng/mL IL-4 and 7 M BAY11-7082, pulsed with mBSA, then washed. Mice were injected s.c. into the tail base with 0.5×10⁶ DC 6 days after the knee joint injections or left untreated (AIA control). 14 days after knee joint injection of mBSA, the same joints were challenged with 100U IL-1β. 2 days later mice previously treated were retreated with 0.5×10⁶ DC (clinical scores shown in FIG. 17). mBSA-specific antibodies in sera were analyzed by ELISA on day 20 and typed with isotype-specific secondary antibodies.

FIG. 18 is a graphical representation illustrating long term suppression of antigen-specific responses by NFkB⁻ DC. Mice were primed with antigen KLH in complete Freund's adjuvant. 7 days later, groups of 5 mice were treated with either 5×10⁵ bone marrow derived DC generated in the presence (Bay DC) or absence of BAY11-7082 and exposed or not to the antigen KLH, or with saline. A final group of mice was left unprimed and untreated. KLH-specific delayed type hypersensitivity was tested at 1, 2, 3 and 8 months in each mouse.

FIG. 19 is a graphical representation showing the restoration of suppression following boosting with antigen. In FIG. 19A, mice were boosted with KLH and complete Freund's adjuvant 1 week after the 3-month DTH analysis, whereas in FIG. 19B mice were boosted with KLH and complete Freund's adjuvant 1 week after the 1- and 3-month DTH analyses.

FIG. 20 is a graphical representation showing that administration of NFkB− donor or recipient DC at the time of allogeneic bone marrow transplantation reduces GVHD mortality. Bone marrow and T cell depleted splenocytes from B6 mice were transplanted into lethally irradiated B6D2F1 recipients (TCD, open squares, n=5), or bone marrow and splenocytes from B6 mice were transplanted into lethally irradiated B6D2F1 recipients in the presence or absence of 5×10⁵ B6 or B6D2F1 bone marrow derived DC (closed squares, n=5 per group, groups pooled). Bone marrow and splenocytes from B6 mice were transplanted into lethally irradiated B6D2F1 recipients in the presence of 5×10⁵ BAY treated B6 or B6D2F1 bone marrow derived DC (open diamonds, n=5 per group, groups pooled). P<0.02 comparing groups receiving DC and BAY-treated DC.

FIG. 21 is a graphical representation showing the prevention of type I diabetes in NOD mice by NF-κB⁻ dendritic cells. Murine DC were generated for 7 days from NOD or proinsulin-NOD bone marrow precursors in the presence of GM-CSF, IL-4 and BAY11-7082, then washed. 12 mice per group were injected s.c. into the tail base with 0.5×10⁶ DC or PBS at the age of 4 weeks or left untreated. Mice were tested weekly for presence of glycosuria. Glycosuric mice were tested by glucometer for blood glucose. Diabetes was diagnosed if blood glucose was greater than 12, on 2 consecutive occasions.

FIG. 22 is a graphical representation showing the phenotype of NF-κB⁻ dendritic cells is reproducible with different NF-κB inhibitors. Dendritic cells were generated from human monocytes for 48 hours in the presence of GM-CSF, IL-4 and in the presence or absence of either 10 μM BAY11-7082 or 2.5 or 10 μM PSI (N-benzyloxycarbonyl-Ile-Glu(O-tert-butyl)-Ala-leucinal). Cells were then stained with anti-CD40, anti-CD86, anti-HLA-DR and anti-MHC class I mAb (green lines) and compared with isotype control staining (purple) by flow cytometry.

FIG. 23 is a graphical representation showing that bone marrow macrophages also induce antigen-specific tolerance. Dendritic cells or macrophages were generated from bone marrow precursors in the presence of growth factors and 10 μM Bay11-7082 then washed and exposed to the antigen KLH. Recipient mice (5 per group) were primed with KLH in CFA, then administered 5×10⁵ DC or macrophages subcutaneously, or no treatment. One group was not primed with KLH in CFA. KLH-specific delayed type hypersensitivity responses were measured on all groups 4 days after administration of antigen presenting cells.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “about” is used herein to refer to conditions (e.g., amounts, concentrations, time etc) that vary by as much as 30%, preferably by as much as 20%, and more preferably by as much as 10% to a specified condition.

The term “anergy” as used herein refers to a suppressed response, or a state of non-responsiveness, to a specified antigen or group of antigens by an immune system. For example, T lymphocytes and B lymphocytes are anergic when they cannot respond to their specific antigen under optimal conditions of stimulation.

By “antigen” is meant all, or part of, a protein, peptide, or other molecule or macromolecule capable of eliciting an immune response in a vertebrate animal, preferably a mammal. Such antigens are also reactive with antibodies from animals immunised with said protein, peptide, or other molecule or macromolecule.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

Reference herein to “a pellet and/or functional activity” in the context of a protein produced by a specified cell is to be taken in its broadest sense and includes a level and/or functional activity of the protein that is produced in a single cell or in a plurality or population of cells. In the latter case, therefore, it will be understood that the phrase will encompass a mean level and/or functional activity of the protein produced by a plurality or population of cells.

By “autologous” is meant something (e.g., cells, tissues etc) derived from the same organism.

The term “allogeneic” as used herein refers to cells, tissues, organisms etc that are of different genetic constitution.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, “culturing”, “culture” and the like refer to the set of procedures used in vitro where a population of cells (or a single cell) is incubated under conditions which have been shown to support the growth or maintenance of the cells in vitro. The art recognises a wide number of formats, media, temperature ranges, gas concentrations etc. which need to be defined in a culture system. The parameters will vary based on the format selected and the specific needs of the individual who practices the methods herein disclosed. However, it is recognised that the determination of culture parameters is routine in nature.

By “effective amount”, in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “expression vector” is meant any autonomous genetic element capable of directing the synthesis of a protein encoded by the vector. Such expression vectors are known by practitioners in the art.

The term “gene” is used in its broadest context to include both a genomic DNA region corresponding to the gene as well as a cDNA sequence corresponding to exons or a recombinant molecule engineered to encode a functional form of a product.

Reference herein to “immuno-active” includes reference to any interaction, reaction, or other form of association between molecules and in particular where one of the molecules is, or mimics, a component of the immune system.

By “isolated” is meant material that is substantially or essentially flee from components that normally accompany it in its native state.

By “modulating” is meant increasing or decreasing, either directly or indirectly, the immune response of an individual.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The term “patient” refers to patients of mammalian, especially human, or other animal origin and includes any individual it is desired to examine or treat using the methods of the invention. However, it will be understood that “patient” does not imply that symptoms are present. Suitable animals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, reptiles, avians, fish).

By “pharmaceutically-acceptable carrier-” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “regulatory lymphocyte” is meant a lymphocyte which is involved in controlling responses and actions of other cells, especially of other immune cells such as B lymphocytes and T helper lymphocytes.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants.

2. Antigen-Presenting Cells

The present invention is predicated in part on the determination that an antigen-presenting cell that produces CD40, or its equivalent, at a level and/or functional activity that is lower than that produced by an activated dendritic cell, is a potent modulator of immune responses and is especially useful not only for preventing priming of immunity but also for suppressing a previously primed immune response to a specified antigen or group of antigens. In some embodiments, the antigen-presenting cell cannot be induced to express CD40, or its equivalent, at an equivalent level and/or functional activity as that produced by an activated antigen presenting cell. In some embodiments, the antigen-presenting cell cannot be induced to express CD40, or its equivalent, at a higher level and/or functional activity than that produced by an immature antigen-presenting cell. In some embodiments, the antigen-presenting cell is other than a B lymphocyte. Advantageously, the antigen-presenting cell produces CD40, or its equivalent, at a level and/or functional activity that is less than about 50%, 40%, 30%, 20%, 10%, 5%, 4%, 3%, 2%, 1%, 0.5%, 0.1%, of that produced by a mature or activated dendritic cell. In some embodiments, the antigen-presenting cell produces CD40, or its equivalent, at a level and/or functional activity that is less than about 50%, 40%, 30%. 20%, 10%, 5%, 4%, 3%, 2%, 1%, 0.5%, 0.1%, of that produce activated dendritic cell. In a preferred embodiment of this type, the antigen-presenting cell produces CD40, or its equivalent, at a level and/or functional activity that is less than that produced by an immature dendritic cell. In preferred embodiments, the antigen-presenting cell is further characterised by being non proliferating; and expressing one or both of a class I and a class U MHC determinant. The antigen-presenting cell preferably encompasses both professional and facultative types of antigen-presenting cells. For example, professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Examples of facultative antigen-presenting cells include but are not limited to activated T cells, astrocytes, follicular cells, endothelium and fibroblasts. In a preferred embodiment, the antigen-presenting cell is selected from monocytes, macrophages, B lymphocytes, cells of myeloid lineage, dendritic cells or Langerhans cells. In an especially preferred embodiment, the antigen-presenting cell expresses CD11c and includes a dendritic cell.

The antigen-presenting cell is preferably further characterised by producing NF-κB or component thereof, especially RelB, at a level and/or functional activity that is less than about 50%, 40%, 30%, 20%, 10%, 5%, 4%, 3%, 2%, 1%, 0.5%, 0.1%, of that produced by a mature or activated dendritic cell. In a preferred embodiment of this type, the antigen-presenting cell produces NF-κB or component thereof, especially RelB, at a level and/or functional activity that is lower than that produced by an immature dendritic cell (Thompson et al., 2002). In some embodiments, the antigen-presenting cell (e.g., dendritic cell) cannot be induced to express NF-κB or component thereof, at a higher level and/or functional activity than an immature antigen presenting cell (e.g., an immature dendritic cell).

Suitably, the antigen-presenting cell is further characterised by expressing an immunostimulatory molecule, such as CD86 or CD80, or their equivalents. In a preferred embodiment of this type, the immunostimulatory molecule is CD86 or its equivalent, and is suitably produced at a level and/or functional activity which is suitably at least about 1%, 5%, 10%, 20%, 30%, 40%, 50%, 60%, 70%, 80%, 90%, 100%, of that produced by an activated dendritic cell. In certain preferred embodiments, the antigen-presenting cell is further characterised by presenting an antigen, which is preferably in the context of MHC molecules expressed by the cell, and causing T lymphocytes to exhibit anergy to that antigen by contact of the cell with the lymphocytes.

The present invention also contemplates an isolated and preferably purified population of antigen-presenting cells as broadly described above. Numerous techniques are known to practitioners in the art for isolating and/or purifying cellular populations, including the use of surgical removal of tissue, flow cytometry techniques such as fluorescence-activated cell sorting (FACS), immunoaffinity separation (e.g., magnetic bead separation such as Dynabead™ separation), density separation (e.g., metrizamide, Percoll™, or Ficoll™ gradient centrifugation), and cell-type specific density separation.

In one embodiment, an antigen-presenting cell according to the present invention is obtained by contacting a precursor of the antigen-presenting cell with an NF-κB inhibitor for a time and under conditions sufficient to differentiate an antigen-presenting cell from the precursor and to inhibit or otherwise reduce the level and/or functional activity of NF-κB in said cell. Alternatively, the antigen-presenting cell of the invention can be produced by contacting an antigen-presenting cell, or its precursor, with an inhibitor of CD40, or its equivalent, for a time and under conditions sufficient to produce a modified antigen-presenting cell that produces CD40, or its equivalent, at a level and/or functional activity that is reduced or abrogated relative to that of said antigen-presenting cell or its precursor. Optionally, the antigen-presenting cell, or its precursor, or the modified antigen-presenting cell, is contacted with an agent that increases the level and/or functional activity of an immunostimulatory molecule, such as CD86 or CD80, or their equivalents (e.g., a polynucleotide from which the immunostimulatory molecule can be expressed), for a time and under conditions sufficient to enhance or otherwise elevate the level and/or functional activity of the immunostimulatory molecule.

2.1 Sources of Antigen Presenting Cell Precursors and Methods for their Differentiation into Antigen-Presenting Cells

Antigen-presenting cell precursors can be isolated by methods known to those of skill in the art. The source of precursor will differ depending upon the antigen-presenting cell required for modulating a specified immune response. In this context, the antigen-presenting cell can be selected from include dendritic cells, macrophages, monocytes and other cells of myeloid lineage. Typically, precursors of antigen-presenting cells can be isolated from any tissue, but are most easily isolated from blood, cord blood or bone marrow (Sorg et al., 2001; Zheng et al., 2000). It is also possible to obtain suitable precursors from diseased tissues such as rheumatoid synovial tissue or fluid following biopsy or joint tap (Thomas et al., 1994a; Thomas et al., 1994b). Other examples include, but are not limited to liver, spleen, heart, kidney, gut and tonsil (Lu et al., 1994; McIlroy et al., 2001; Vremec et al., 2000) (Hart and Fabre, 1981; Hart and McKenzie, 1988; Pavli et al., 1990).

Leukocytes isolated directly from tissue provide a major source of antigen-presenting cell precursors. Typically, these precursors can only differentiate into antigen-presenting cells by culturing in the presence or absence of various growth factors. According to the practice of the present invention, the antigen-presenting cells may be so differentiated from crude mixtures or from partially or substantially purified preparations of precursors. Leukocytes can be conveniently purified from blood or bone marrow by density gradient centrifugation using, for example, Ficoll Hypaque which eliminates neutrophils and red cells (peripheral blood mononuclear cells or PBMCs), or by ammonium chloride lysis of red cells (leukocytes or white blood cells). Many precursors of antigen-presenting cells are present in peripheral blood as non-proliferating monocytes, which can be differentiated into specific antigen-presenting cells, including macrophages and dendritic cells, by culturing in the presence of specific cytokines.

Tissue-derived precursors such as precursors of tissue dendritic cells or of Langerhans cells are typically obtained by mincing tissue (e.g., basal layer of epidermis) and digesting it with collagenase or dispase followed by density gradient separation, or selection of precursors based on their expression of cell surface markers. For example, Langerhans cell precursors express CD1 molecules as well as HLA-DR and can be purified on this basis.

In one embodiment, the antigen-presenting cell precursor is a precursor of macrophages. Generally these precursors can be obtained from monocytes of any source and can be differentiated into macrophages by prolonged incubation in the presence of medium and macrophage colony stimulating factor (M-CSF) (Erickson-Miller et al., 1990; Metcalf and Burgess, 1982).

In another embodiment, the antigen presenting cell precursor is a precursor of Langerhans cells. Usually, Langerhans cells can be generated from human monocytes or CD34⁺ bone marrow precursors in the presence of granulocyte/macrophage colony-stimulating factor (GM-CSF), IL-4/TNFα and TGFβ (Geissmann et al., 1998; Strobl et al., 1997a; Strobl et al., 1997b; Strobl et al., 1996)

In a preferred embodiment, the antigen-presenting cell precursor is a precursor of dendritic cells. Several potential dendritic cell precursors can be obtained from peripheral blood, cord blood or bone marrow. These include monocytes, CD34+stem cells, granulocytes, CD33⁺CD11c⁺ DC precursors, and committed myeloid progenitors—described below.

Monocytes. Monocytes can be purified by adherence to plastic for 1-2 h in the presence of tissue culture medium (e.g., RPMI) and serum (e.g., human or foetal calf serum), or in serum-free medium (Anton et al., 1998; Araki et al., 2001; Mackensen et al., 2000; Nestle et al., 1998; Romani et al., 1996; Thumer et al., 1999). Monocytes can also be elutriated from peripheral blood (Garderet et al., 2001). Monocytes can also be purified by immunoaffinity techniques, including immunomagnetic selection, flow cytometric sorting or panning (Araki et al., 2001; Battye and Shortman, 1991), with anti-CD14 antibodies to obtain CD14^{hi} cells. The numbers (and therefore yield) of circulating monocytes can be enhanced by the in vivo use of various cytokines including GM-CSF (Groopman et al., 1987; Hill et al., 1995). Monocytes can be differentiated into dendritic cells by prolonged incubation in the presence of GM-CSF and IL-4 (Romani et al., 1994; Romani et al., 1996). A combination of GM-CSF and IL-4 at a concentration of each at between about 200 to about 2000 U/mL, more preferably between about 500 to about 1000 U/mL and even more preferably between about 800 U/mL (GM-CSF) and 1000 U/mL (IL-4) produces significant quantities of immature dendritic cells, i.e., antigen-capturing phagocytic dendritic cells. Other cytokines which promote differentiation of monocytes into antigen-capturing phagocytic dendritic cells include, for example, IL-13.

CD34⁺ stem cells. Dendritic cells can also be generated from CD34⁺ bone marrow derived precursors in the presence of GM-CSF, TNFα±stem cell factor (SCF, c-kitL), or GM-CSF, IL-4±flt3L (Bai et al., 2002; Chen et al., 2001; Loudovaris et al., 2001). CD34⁺ cells can be derived from a bone marrow aspirate or from blood and can be enriched as for monocytes using, for example, immunomagnetic selection or immunocolumns (Davis et al., 1994). The proportion of CD34⁺ cells in blood can be enhanced by the in vivo use of various cytokines including (most commonly) G-CSF, but also flt3L and progenipoietin (Fleming et al., 2001; Pulendran et al. 2000; Robinson et al., 2000).

Other myeloid progenitors. DC can be generated from committed early myeloid progenitors in a similar fashion to CD34⁺ stem cells, in the presence of GM-CSF and IL-4/TNF. Such myeloid precursors infiltrate many tissues in inflammation, including rheumatoid arthritis synovial fluid (Santiago-Schwarz et al., 2001). Expansion of total body myeloid cells including circulating dendritic cell precursors and monocytes, can be achieved with certain cytokines, including flt-3 ligand, granulocyte colony-stimulating factor (G-CSF) or progenipoietin (pro-GP) (Fleming et al., 2001; Pulendran et al., 2000; Robinson et al., 2000). Administration of such cytokines for several days to a human or other mammal would enable much larger numbers of precursors to be derived from peripheral blood or bone marrow for in vitro manipulation. Dendritic cells can also be generated from peripheral blood neutrophil precursors in the presence of GM-CSF, IL-4 and TNFα (Kelly et al., 2001; Oehler et al., 1998). It should be noted that dendritic cells can also be generated, using similar methods, from acute myeloid leukemia cells (Oehler et al., 2000).

Tissue DC precursors and other sources of APC precursors. Other methods for DC generation exist from, for example, thymic precursors in the presence of IL-3+/−GM-CSF, and liver DC precursors in the presence of GM-CSF and a collagen matrix. Transformed or immortalised dendritic cell lines may be produced using oncogenes such as v-myc as for example described by (Paglia et al., 1993) or by myb (Banyer and Hapel, 1999; Gonda et al., 1993).

Circulating DC precursors. These have been described in human and mouse peripheral blood. One can also take advantage of particular cell surface markers for identifying suitable dendritic cell precursors. Specifically, various populations of dendritic cell precursors can be identified in blood by the expression of CD11c and the absence or low expression of CD14, CD19, CD56 and CD3 (O'Doherty et al., 1994; O'Doherty et al., 1993). These cells can also be identified by the cell surface markers CD13 and CD33 (Thomas et al., 1993b). A second subset, which lacks CD14, CD19, CD56 and CD3, known as plasmacytoid dendritic cell precursors, does not express CD11c, but does express CD123 (IL-3R chain) and HLA-DR (Farkas et al., 2001; Grouard et al., 1997; Rissoan et al., 1999). Most circulating CD11c⁺ dendritic cell precursors are HLA-DR⁺, however some precursors may be HLA-DR−. The lack of MHC class II expression has been clearly demonstrated for peripheral blood dendritic cell precursors (del Hoyo et al., 2002).

Optionally, CD33⁺CD14^{-/lo} or CD11c⁺HLA-DR⁺, lineage marker-negative dendritic cell precursors described above can be differentiated into more mature antigen-presenting cells by incubation for 18-36 h in culture medium or in monocyte conditioned medium (Thomas et al., 1993b; Thomas and Lipsky, 1994) (O'Doherty et al., 1993). Alternatively, following incubation of peripheral blood non-T cells or unpurified PBMC, the mature peripheral blood dendritic cells are characterised by low density and so can be purified on density gradients, including metrizamide and Nycodenz (Freudenthal and Steinman, 1990; Vremec and Shortman, 1997), or by specific monoclonal antibodies, such as but not limited to the CMRF-44 mAb (Fearnley et al., 1999; Vuckovic et al., 1998). Plasmacytoid dendritic cells can be purified directly from peripheral blood on the basis of cell surface markers, and then incubated in the presence of IL-3 (Grouard et al., 1997; Rissoan et al., 1999). Alternatively, plasmacytoid DC can be derived from density gradients or CMRF-44 selection of incubated peripheral blood cells as above.

In general, for dendritic cells generated from any precursor, when incubated in the presence of activation factors such as monocyte-derived cytokines, lipopolysaccharide and DNA containing CpG repeats, cytokines such as TNF-α, IL-6, IFN-α, IL-1β, necrotic cells, readherence, whole bacteria, membrane components, RNA or polyIC, immature dendritic cells will become activated (Clark, 2002; Hacker et al., 2002; Kaisho and Akira, 2002; Koski et al., 2001). This process of dendritic cell activation is inhibited in the presence of NF-κB inhibitors (O'Sullivan and Thomas, 2002).

2.2 Modulatory Agents for Producing the Subject Antigen-Presenting Cells

In one embodiment, an antigen-presenting cell of the present invention is produced by contacting a precursor of an antigen-presenting cell, such as but not limited to the precursors mentioned above, with an NF-κB inhibitor. Numerous inhibitors of NF-κB are known to those of skill in the art. The inhibitors may act directly on NF-κB, or indirectly via another entity that regulates the level and/or functional activity of NF-κB. Indirect inhibitors include, but are not limited to, inhibitors of proteolysis and inhibitors of nuclear translocation of NF-κB. The inhibitor of nuclear translocation of NF-κB includes, but is not limited to, deoxyspergualin (Tapper, et al., 1995, J Immunol. 155:2427-9436) or derivatives or analogues of deoxyspergualin including, for example, methyl-deoxyspergualin, a deoxyspergualin analog lacking a chiral centre (e.g., LF 08-0299) (Andoins et al., 1996, Transplantation 62:1543-1549) and the derivatives or analogues identified in U.S. Pat. No. 4,518,532, U.S. Pat. No. 4,518,532, U.S. Pat. No. 4,252,299, U.S. Pat. No. 4,956,504, U.S. Pat. No. 5,162,581, U.S. Pat. No. 5,476,870, U.S. Pat. No. 5,637,613, WO 96/24579, EP 600762, EP 669316, EP 7433000, EP 765866, and EP 755380. The inhibitor of proteolysis is preferably but not exclusively a proteosome inhibitor such as PSI (Traechner, et al., 1994, EMBO J. 13:5433-5441; Griscavage, et al., 1996, Proc. Natl. Acad. Sci. USA 93:3308-3312; Bondeson, et al., 1999, J Immunol. 162:2939-2945), ALLN (Jobin, et al., 1998, Hepatology 27:1285-1295), lactacystin (Delic, et al., 1998, Br. J. Cancer 77:1103-1107), MG-132 (Jobin, et al. supra), C-LFF and calpain inhibitors (Neauparfant and Hiscott, 1996, Cytokine & Growth Factor Reviews 7:175-190) and CVT-134 (Lum, et al., 1998, Biochem. Pharmacol 55:1391-1397). Other indirect NF-κB inhibitors include: caffeic acid phenethyl ester (Natarajan, et al., 1992, Proc. Natl. Acad. Sci. USA 93:9090-9095); pyrrolidine dithiocarbonate (Schreck, et al., 1992, J. Exp. Med. 175:1181-1194); lovastatin (Guijarro, et al., 1996, Nephrol. Dial. Transplant 11:990-996); aselastine HCL (Yoneda, 1997, Japan. J. Pharmacol. 73:145-153); tepaxalin (Kazmi, et al., 1995, J. Cell. Biochem. 57:299-310); (−)-epi gallocatechin-3-gallate (Lin & Lin, 1997, Mol. Pharmacol. 52:465-472); phenyl-N-tert-butylnitrone (Kotake, et al., 1998, Biochem. Biopsies. Acta 1446:77-84; quercetin (Sato, et al., 1997, J. Rheumatol. 24:1680-1684); cucumin (Chan, 1998, Biochem. Pharmacol. 55:965-973); or E330 (Goto, et al., 1996, Mol. Pharmacol. 49:860-873). The p38 MAP kinase inhibitor, SB203580, also blocks nuclear RelB translocation of LPS-treated monocyte-derived dendritic cells, through an unknown mechanism that is presumably indirect. In a preferred embodiment, the indirect inhibitor of NF-κB is an inhibitor of IκB degradation including, but not limited to, inhibitors of IκB phosphorylation, IκB ubiquitination and proteolytic degradation of IκB, for example, by the proteosome.

Alternatively, the NF-κB inhibitor is a direct inhibitor of NF-κB, i.e., acts directly on the level (quantity), cellular location or activity of NF-κB. For example, the inhibitor may be a naturally occurring regulator of NF-κB that interacts directly with NF-κB, such as an IκB, especially IκBα, as for example described by Makarov (1997, Gene Therapy 4:846-852) and in PCT/GB98/02753. For example, Bondeson et al. (1999, Proc. Natl. Acad. Sci. USA 96:5668-5673) describe an IκB-encoding adenovirus. Other inhibitors of NF-κB include inhibitors of nuclear localisation of NF-κB, inhibitors of DNA binding of NF-κB as well as antisense nucleic acid molecules or oligonucleotides, which are complementary or encode at least a portion of any of the NF-κB subunits, e.g., p50, p65, RelB. Preferably, the inhibitor of NT-κB is an inhibitor of RelB or p50. Such an inhibitor may be a ribozyme which selectively destroys RNA encoding NF-κB, or an antisense molecule which prevents transcription of NF-κB or an antigen-binding molecule (e.g., antibody, Fv, Fab, Fab′ and F(ab′)₂ immunoglobulin fragments or other synthetic antigen-binding molecules such as synthetic stabilised Fv fragments, dabs, minibodies and the like) which blocks NF-κB action (O'Sullivan et al., 2000).

Especially preferred inhibitors of NF-κB are inhibitors of IκB phosphorylation, such as BAY 11-7082 (BioMol, Plymouth Meeting, Pa.). BAY 11-7082 has been shown to block TNF-α-stimulated NF-κB translocation through inhibition of IκB phosphorylation (Pierce et al., 1997).

In another embodiment, an antigen-presenting cell of the present invention is produced by contacting a differentiated antigen-presenting cell, or a precursor thereof, as for example described in Section 2.1, with a CD40 inhibitor for a time and under conditions sufficient to produce a modified antigen-presenting cell that produces CD40, or its equivalent, at a reduced or abrogated level and/or functional activity relative to that of said antigen-presenting cell or its precursor. The CD40 inhibitor can be an indirect inhibitor but is preferably a direct inhibitor of CD40, i.e., acts directly on the level (quantity), cellular location or activity of CD40. Examples of such direct inhibitors include antisense nucleic acid molecules or oligonucleotides, which are complementary or encode at least a portion of CD40, ribozymes which selectively destroy CD40-encoding RNA, antigen-binding molecules which block CD40 activation and CD40 antagonists. Optionally, the differentiated antigen-presenting cell, or a precursor thereof, as for example described in Section 2.1, or the modified antigen-presenting cell as described above is further contacted with an activator or inducer of an immunostimulatory molecule, especially of CD86 or CD80, or their equivalents, for a time and under conditions sufficient to enhance or otherwise elevate the level and/or functional activity of the immunostimulatory molecule. For example, the activator or inducer can be a transcriptional activator, which enhances the expression of the immunostimulatory molecule, or an expression vector from which the immunostimulatory molecule is expressible.

The amount of modulator (e.g., NF-κB inhibitor) to be placed in contact with the antigen-presenting cell precursors can be determined empirically by persons of skill in the art. For example, the precursor is cultured with an NF-κB inhibitor for the duration of the process of dendritic cell differentiation from its precursors, typically for about 1 to 120 hours, preferably for about 4 to 36 hours for peripheral blood dendritic cell precursors other than monocytes and tissue precursors, and preferably between about 48 to 120 hours and up to 168 hours for monocyte precursors, and greater than 108 hours (7-10 days) for CD34⁺ and other committed myeloid precursors. Thus, cells and inhibitors are incubated in the presence of one or more factors required for the differentiation of the precursor to the antigen-presenting cell of interest.

In especially preferred embodiments, dendritic cell precursors, which are preferably derived from bone marrow, are cultured in the presence of dendritic cell growth factors and an NF-κB inhibitor, especially BAY 11-7082, for about 6-10 days, or monocyte precursors which are preferably derived from peripheral blood are incubated in the presence of dendritic cell growth factors and an NFκB inhibitor, especially BAY 11-7082, for about 2-5 days.

3. Antigen-Specific Antigen-Presenting Cells

The antigen-presenting cells of the present invention are useful for modulating an immune response, and are especially useful for inducing a tolerogenic response including the induction of an anergic response, and the suppression of a future or existing immune response, to one or more target antigens. Antigen-specific antigen-presenting cells can be produced by contacting an antigen-presenting cell of the invention with at least one antigen that corresponds to a specified target antigen, or with a polynucleotide from which the antigen is expressible, for a time and under conditions sufficient for the antigen or a processed form thereof to be presented by the antigen-presenting cell. Alternatively, a precursor of the antigen-presenting cell can be co-cultured with an NF-κB inhibitor, together with the antigen, or with a polynucleotide from which the antigen is expressible, for a time and under conditions sufficient for an antigen-presenting cell to differentiate from the precursor and for the level and/or functional activity of NF-κB in the antigen-presenting cell to be abrogated or otherwise reduced and for the antigen, or processed form thereof, to be presented by the antigen-presenting cell.

A variety of possible antigens exist for which modulation of an immune response, especially the induction of a tolerogenic immune response, and more especially the induction of antigen-specific lymphocyte anergy, may be important. Both alloantigens and self antigens are presented in the context of MHC. Other antigens for which such modulation of an immune response may be important include soluble antigens, e.g., soluble proteins or fragments of insoluble complexes, particulate antigens, e.g., bacteria or parasites, and allergens. Thus, exemplary antigen which may be used in the practice of the present invention include, but are not limited to, self antigens that are targets of autoimmune responses, allergens and transplantation antigens. Examples of self antigens include, but are not restricted to, lupus autoantigen, Smith, Ro, La, U1-RNP, fibrillin (scleroderma), GAD65 (diabetes related), insulin, myelin basic protein, histones, PLP, collagen, glucose-6-phosphate isomerase, citrullinated proteins and peptides, thyroglobulin, various tRNA synthetases, acetyl choline receptor (AchR), MOG, proteinase-3, myeloperoxidase etc. Examples of allergens include, but are not limited to, Fel d 1 (i.e., the feline skin and salivary gland allergen of the domestic cat Felis domesticus, the amino acid sequence of which is disclosed International Publication WO 91/06571), Der p I, Der p II, Der fI or Der fII (i.e., the major protein allergens from the house dust mite dermatophagoides, the amino acid sequence of which is, disclosed in International Publication WO 94/24281). Other allergens may be derived, for example from the following: grass, tree and weed (including ragweed) pollens; fungi and moulds; foods such as fish, shellfish, crab, lobster, peanuts, nuts, wheat gluten, eggs and milk; stinging insects such as bee, wasp, and hornet and the chirnomidae (non-biting midges); other insects such as the housefly, fruitfly, sheep blow fly, screw worm fly, grain weevil, silkworm, honeybee, non-biting midge larvae, bee moth larvae, mealworm, cock-roach and larvae of Tenibrio molitor, beetle; spiders and mites, including the house dust mite; allergens found in the dander, urine, saliva, blood or other bodily fluid of mammals such as cat, dog, cow, pig, sheep, horse, rabbit, rat, guinea pig, mouse and gerbil; airborne particulates in general; latex; and protein detergent additives. Transplantation antigens can be derived from donor cells or tissues or from the donor antigen-presenting cells bearing MHC loaded with self antigen in the absence of exogenous antigen.

3.1 Preparation of Antigen

The antigen(s) may be isolated from a natural source or may be prepared by recombinant techniques as is known in the art. For example, peptide antigens can be eluted from the MHC and other presenting molecules of antigen-presenting cells obtained from a cell population or tissue for which a modified immune response is desired. The eluted peptides can be purified using standard protein purification techniques known in the art (Rawson et al., 2000; Smithers et al., 2002). If desired, the purified peptides can be sequenced and synthetic versions of the peptides produced using standard protein synthesis techniques as for example described below. Alternatively, crude antigen preparations can be produced by isolating a sample of a cell population or tissue for which a modified immune response is desired, and either lysing the sample or subjecting the sample to conditions that will lead to the formation of apoptotic cells (e.g., irradiation with ultra violet or with gamma rays, spiral infection, cytokines or by depriving cells of nutrients in the cell culture medium, incubation with hydrogen peroxide, or with drugs such as dexamethasone, ceramide chemotherapeutics and anti-hormonal agents such as Lupron or Tamoxifen). The lysate or the apoptotic cells can then be used as a source of crude antigen for contact with the antigen-presenting cells.

When the antigen is known, it may be conveniently prepared in recombinant form using standard protocols as for example described in: Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Typically, an antigen may be prepared by a procedure including the steps of (a) providing an expression vector from which the target antigen or analogue or mimetic thereof is expressible; (b) introducing the vector into a suitable host cell; (c) culturing the host cell to express recombinant polypeptide from the vector; and (d) isolating the recombinant polypeptide.

In general, the expression vector will comprise an antigen-encoding polynucleotide which is operably connected to a regulatory polynucleotide. The antigen-encoding polynucleotide can be constructed from any suitable parent polynucleotide that codes for an antigen that corresponds to the target antigen of interest. The parent polynucleotide is suitably a natural gene or portion thereof. However, it is possible that the parent polynucleotide is not naturally-occurring but has been engineered using recombinant techniques. The regulatory polynucleotide suitably comprises transcriptional and/or translational control sequences, which will generally be appropriate for the host cell used for expression of the antigen-encoding polynucleotide. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the host cell to be introduced or may be derived from an alternative source, where the region is functional in the host cell.

The expression vector may also comprise a 3′ non-translated sequence, which usually refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognised by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

In a preferred embodiment, the expression vector further contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners also include within their scope “epitope tags”, which are usually short peptide sequences for which a specific antibody is available. Well known examples of epitope tags for which specific monoclonal antibodies are readily available include c-Myc, influenza virus haemagglutinin and FLAG tags.

The step of introducing the expression vector into the host cell may be effected by any suitable method including transfection, transduction of viral vectors, including adenoviral, modified lentiviral and other retroviral vectors, and transformation, the choice of which will be dependent on the host cell employed. Such methods are well known to those of skill in the art.

Recombinant polypeptides of the invention may be produced by culturing a host cell transformed with the expression vector under conditions appropriate for protein expression, which will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

Alternatively, the antigen can be synthesised using solution synthesis or solid phase synthesis as described, for example, by Atherton and Sheppard (Solid Phase Peptide Synthesis: A Practical Approach, IRL Press at Oxford University Press, Oxford, England, 1989) or by Roberge et al. (1995, Science 269: 202).

The delivery of exogenous antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang, 1994, Immunomethods, 4:229-235), osmotic lysis of pinosomes after pinocytic uptake of soluble antigen (Moore et al., 1988, Cell, 54:777-785), coupling of antigens to potent adjuvants (Aichele et al., 1990, J. Exp. Med., 171: 1815-1820; Gao et al., 1991, J. Immunol., 147: 3268-3273; Schulz et al., 1991, Proc. Natl. Acad. Sci. USA, 88: 991-993; Kuzu et al., 1993, Euro. J. Immunol., 23: 1397-1400; and Jondal et al., 1996, Immunity 5: 295-302) and apoptotic cell delivery of antigen (Albert et al. 1998, Nature 392:86-89; Albert et al. 1998, Nature Med. 4:1321-1324; and in International Publications WO 99/42564 and WO 01/85207). Recombinant bacteria (eg. E. coli) or transfected host mammalian cells may be pulsed onto dendritic cells (as particulate antigen, or apoptotic bodies respectively) for antigen delivery. Such a delivery system might be logically combined with a substance for inhibiting NF-κB, such as a plasmid encoding dominant negative IΛBα (Pai et al., 2002). Recombinant chimeric virus-like particles (VLPs) have also been used as vehicles for delivery of exogenous heterologous antigen to the MHC class I processing pathway of a dendritic cell line (Bachmann et al., 1996, Eur. J. Immunol., 26(11): 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see e.g., Cox and Coulter, 1997, Vaccine 15(3): 248-256 and U.S. Pat. No. 6,352,697), phospholipases (see, e.g., Camilli et al., 1991, J. Exp. Med. 173: 751-754), pore-forming toxins (e.g., an alpha-toxin), natural cytolysins of gram-positive bacteria, such as listeriolysin O (LLO, e.g., Mengaud et al., 1988, Infect. Immun. 56: 766-772 and Portnoy et al., 1992, Infect. Immun. 60: 2710-2717) streptolysin O (SLO, e.g., Palmer et al., 1998, Biochemistry 37(8): 2378-2383) and perfringolysin O (PFO, e.g., Rossjohn et al., Cell 89(5): 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al., Infect. Immun. 1992, 60: 2710-2717).

The cytolysin may be provided together with a pre-selected antigen in the form of a single composition or may be provided as a separate composition, for contacting the antigen-presenting cells. In one embodiment, the cytolysin is fused or otherwise linked to the antigen, wherein the fusion or linkage permits the delivery of the antigen to the cytosol of the target cell. In another embodiment, the cytolysin and antigen are provided in the form of a delivery vehicle such as, but not limited to, a liposome or a microbial delivery vehicle selected from virus, bacterium, or yeast. Preferably, when the delivery vehicle is a microbial delivery vehicle, the delivery vehicle is non-virulent. In a preferred embodiment of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory polynucleotide which expresses the cytolysin in the bacterium, and a second polynucleotide encoding one or more pre-selected antigens. Non-secreted cytolysins may be provided by various mechanisms, e.g., absence of a functional signal sequence, a secretion incompetent microbe, such as microbes having genetic lesions (e.g., a functional signal sequence mutation), or poisoned microbes, etc. A wide variety of nonvirulent, non-pathogenic bacteria may be used; preferred microbes are relatively well characterised strains, particularly laboratory strains of E. coli, such as MC4100, MC1061, DH5.alpha., etc. Other bacteria that can be engineered for the invention include well-characterised, nonvirulent, non-pathogenic strains of Listeria monocytogenes, Shigella flexneri, mycobacterium, Salmonella, Bacillus subtilis, etc. In a particular embodiment, the bacteria are attenuated to be non-replicative, non-integrative into the host cell genome, and/or non-motile inter- or intra-cellularly.

The delivery vehicles described above can be used to deliver one or more antigens to virtually any antigen-presenting cell capable of endocytosis of the subject vehicle, including phagocytic and non-phagocytic antigen-presenting cells. In embodiments when the delivery vehicle is a microbe, the subject methods generally require microbial uptake by the target cell and subsequent lysis within the antigen-presenting cell vacuole (including phagosomes and endosomes).

3.2 Delivery of Antigen into Antigen-Presenting Cells

The amount of antigen to be placed in contact with antigen-presenting cells can be determined empirically by persons of skill in the art. Typically antigen-presenting cells are incubated with antigen for about 1 to 6 hr at 37° C., although it is also possible to expose antigen-presenting cells to antigen for the duration of incubation with growth factors and inhibitor. Usually, for purified antigens and peptides, 0.1-10 μg/mL is suitable for producing antigen-specific antigen-presenting cells dendritic cells are exposed to apoptotic bodies in approximately 1:1 ratio, and bacteria (Albert et al., 1998; Corinti et al., 1999). The antigen should be exposed to the antigen-presenting cells for a period of time sufficient for those cells to internalise the antigen. The time and dose of antigen necessary for the cells to internalise and present the processed antigen may be determined using pulse-chase protocols in which exposure to antigen is followed by a washout period and exposure to a read-out system e.g., antigen reactive T cells. Once the optimal time and dose necessary for cells to express processed antigen on their surface is determined, a protocol may be used to prepare cells and antigen for inducing tolerogenic responses. Those of skill in the art will recognise in this regard that the length of time necessary for an antigen-presenting cell to present an antigen may vary depending on the antigen or form of antigen employed, its dose, and the antigen-presenting cell employed, as well as the conditions under which antigen loading is undertaken. These parameters can be determined by the skilled artisan using routine procedures.

In another embodiment, an antigen of interest can be produced inside the antigen-presenting cell by introduction of a suitable expression vector as for example described above. The antigen-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimised polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector for introduction into the antigen-presenting cell will be compatible therewith such that the antigen-encoding polynucleotide is expressible by the cell. For example, expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol 11(2):205-208), Vigna and Naldini (2000, J. Gene Med. 2(5):308-316), Kay, et. al. (2001, Nat. Med. 7(1):33-40), Athanasopoulos, et al. (2000, Int. J Mol. Med. 6(4):363-375) and Walther and Stein (2000, Drugs 60(2):249-271). The expression vector is introduced into the antigen-presenting cell by any suitable means which will be dependent on the particular choice of expression vector and antigen-presenting cell employed. Such means of introduction are well-known to those skilled in the art. For example, introduction can be effected by use of contacting (e.g., in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., Lipofectin®, Lipofectamine™, and the like, supplied by Life Technologies, Gibco BRL, Gaithersburg, Md.).

In another embodiment, the antigen-specific antigen-presenting cells can be obtained by isolating antigen-presenting cell precursors from a cell population or tissue to which modification of an immune response is desired and causing these precursors to differentiate into antigen-presenting cells of the invention using the methods described herein. Typically, some of the isolated precursors will constitutively present antigens or have taken up such antigen in vivo in the issue of interest that are targets or potential targets of an immune response for which tolerisation is desired. In this instance, the delivery of exogenous antigen is not essential. Alternatively, cells may be derived from biopsies of healthy or diseased tissues, lysed or rendered apoptotic and the pulsed onto dendritic cells.

As noted above, the antigen-specific antigen-presenting cells of the invention may be obtained or prepared to contain and/or express one or more antigens by any number of means, such that the antigen(s) or processed form(s) thereof, is (are) presented by those cells for potential modulation of other immune cells, including T lymphocytes and B lymphocytes, and particularly for producing T lymphocytes and B lymphocytes that exhibit anergy to a specified antigen or group of antigens. In an especially preferred embodiment, the subject antigen-specific antigen-presenting cells are useful for producing T lymphocytes that exhibit tolerance/anergy to an antigen or group of antigens. The efficiency of inducing lymphocytes, especially T lymphocytes, to exhibit anergy for a specified antigen can be determined by assaying immune responses to that antigen including, but not limited to, assaying T lymphocyte cytolytic activity in vitro using for example the antigen-specific antigen-presenting cells as targets of antigen-specific cytolytic T lymphocytes (CTL); assaying antigen-specific T lymphocyte proliferation (see, e.g., Vollenweider and Groseurth, 1992, J. Immunol. Meth. 149: 133-135), measuring B cell response to the antigen using, for example, ELISPOT assays, and ELISA assays; interrogating cytokine profiles; or measuring delayed-type hypersensitivity (DTH) responses by test of skin reactivity to a specified antigen (see, e.g., Chang et al. (1993, Cancer Res. 53: 1043-1050). Other methods known to practitioners in the art, which can detect the presence of antigen on the surface of antigen-presenting cells after exposure to the antigen, are also contemplated by the present invention.

The anergy-inducing antigen-presenting cells of the present invention have the capacity to efficiently present an antigen, or processed form thereof, on one or both of MHC class I molecules and MHC class II molecules. For example, the dendritic cells of the invention are capable of presenting antigen, or processed form thereof, on both of MHC class I and class II molecules. In this regard, antigens are acquired by dendritic cells through the exogenous pathway by phagocytosis and have the ability to process exogenous antigen for MHC class I and MHC class II presentation. Accordingly, both CD4⁺ T helper lymphocytes and CTL may be rendered anergic by the antigen-presenting dendritic cells of the invention, and relative proportions can be altered by altering the form in which antigen is provided to the antigen-presenting cell, and the nature of the antigen-presenting cell as discussed above. Moreover, the dendritic cells of the invention can be charged with multiple antigens on multiple MHCs to yield polyclonal or oligoclonal anergy of T lymphocytes.

4. Antigen-Specific Allergic Lymphocytes

The present invention also provides antigen-specific anergic B or T lymphocytes, especially T lymphocytes, which fail to respond in an antigen-specific fashion to representation of the antigen. Moreover, the T lymphocytes actively regulate prior immune responses or subsequent priming to that antigen. The regulation appears to be long lived and is maintained, for example, for at least about 3 months, and preferably years.

In a preferred embodiment, antigen-specific anergic T lymphocytes are produced by contacting an antigen-specific antigen-presenting cell as defined above with a population of T lymphocytes, which may be obtained from any suitable source such as spleen or tonsil/lymph nodes but is preferably obtained from peripheral blood. The T lymphocytes can be used as crude preparations or as partially purified or substantially purified preparations, which are suitably obtained using standard techniques as, for example, described in “Immunochemical Techniques, Part G: Separation and Characterization of Lymphoid Cells” (Meth. in Enzymol. 108, Edited by Di Sabato et al., 1984, Academic Press). This includes rosetting with sheep red blood cells, passage across columns of nylon wool or plastic adherence to deplete adherent cells, immunomagnetic or flow cytometric selection using appropriate monoclonal antibodies as described (Cavanagh et al., 1998; Thomas et al., 1993a).

The preparation of T lymphocytes is contacted with the antigen-specific antigen-presenting cells of the invention for an adequate period of time for inducing anergy in the T lymphocytes to the antigen or antigens presented by those antigen-presenting cells. This period will preferably be at least about 1 day, and up to about 5 days. Generally, the proliferation of anergic T lymphocytes produced after this procedure is short-lived and they produce IL-10 in an antigen-specific manner.

In an especially preferred embodiment, a population of antigen-presenting cell precursors is cultured in the presence of a heterogeneous population of T lymphocytes, which is suitably obtained from peripheral blood, together with an NF-κB inhibitor and an antigen to which a modified immune response is required, or with a polynucleotide from which the antigen is expressible. These cells are cultured for a period of time and under conditions sufficient for: (1) the precursors to differentiate into antigen-presenting cells; (2) the level and/or functional activity of NF-κB in those antigen-presenting cells to be abrogated or otherwise reduced; (3) the antigen, or processed form thereof, to be presented by the antigen-presenting cells; and (4) the antigen-presenting cells to induce a subpopulation of the T lymphocytes to exhibit anergy to the antigen-wherein the subpopulation is characterised by not proliferating and by producing L-10. This can occur using Ficoll-purified PBMC plus antigen plus NF-κB inhibitor since such a preparation contains both dendritic cell precursors and T lymphocytes.

The antigen-specific anergy induced by the antigen-specific antigen presenting cells of the invention involves a mechanism which is distinguishable from certain other forms of non-responsiveness. In accordance with the present invention, the antigen-specific antigen-presenting cells induce one or more types of antigen-specific regulatory lymphocytes, especially regulatory T lymphocytes. Several populations of regulatory T lymphocytes are known to inhibit the response of other (effector) lymphocytes in an antigen-specific manner including, for example, Tr1 lymphocytes, Th3 lymphocytes, Th2 lymphocytes, CD8⁺CD28⁻ regulatory T lymphocytes, natural killer (NK) T lymphocytes and γδ T lymphocytes.

Tr1 lymphocytes can emerge after several rounds of stimulation of human blood T cells by allogeneic monocytes in the presence of IL-10. This subpopulation secretes high levels of IL-10 and moderate levels of TGFβ but little IL-4 or IFNγ (Groux et al., 1997, Nature 389:737-742).

The Th3 regulatory subpopulation refers to a specific subset induced following antigen delivery via the oral (or other mucosal) route. They produce predominantly TGFβ, and only low levels of IL-10, IL-4 or IFNγ, and provide specific help for IgA production (Weiner et al., 2001, Microbes Infect 3:947-954). They are able to suppress both Th1 and Th2-type effector T cells.

The lymphocytes produce high levels of IL-4, IL-5 and IL-10 but low IFNγ and TGFβ. Th2 lymphocytes are generated in response to a relative abundance of IL-4 and lack of IL-12 in the environment at the time of presentation of their cognate peptide ligands (O'Garra and Arai, 2000, Trends Cell Biol 10:542-550). T lymphocyte signalling by CD86 may also be important for generation of Th2 cells (Lenschow et al., 1996, Immunity 5:285-293; Xu et al., 1997, J Immunol 159:4217-4226).

A distinct CD8⁺CD28⁻ regulatory or “suppressor” subset of T lymphocytes can been induced by repetitive antigenic stimulation in vitro. They are MHC class I-restricted, and suppress CD4⁺ T cell responses.

NK T lymphocytes, which express the NK cell marker, CD161, and whose TCR are Vα24JαQ in human and Vα14Jα281 in mouse, are activated specifically by the non-polymorphic CD1d molecule through presentation of a glycolipid antigen (Kawano et al., 1997, Science 278:1626-1629). They have been shown to be immunoregulatory in a number of experimental systems. They are reduced in number in several autoimmune models before disease onset, and can reduce incidence of disease upon passive transfer to non-obese diabetic (NOD) mice. Administration of the glycolipid, α-galactosyl ceramide (α-gal cer), presented by CD1d, also results in accumulation of NKT lymphocytes and amelioration of diabetes in these mice (Naumov et al., 2001, Proc Natl Acad Sci USA 98:13838-13843).

γδ T lymphocytes have been implicated in the downregulation of immune responses in various inflammatory diseases and in the suppression of inflammation associated with induction of mucosal tolerance. The tolerance induced by mucosal antigen was transferable to untreated recipient mice by small numbers of γδ T cells (McMenamin et al., 1995, J Immunol 154:4390-4394; McMenamin et al., 1994, Science 265:1869-1871). Moreover, mucosal tolerance induction was blocked by the administration of the GL3 antibody that blocks γδ T cell function (Ke et al., 1997, J Immunol 158:3610-3618).

Thus, the present invention provides means to generate large quantities of antigen-specific lymphocytes by stimulating lymphocytes with antigen-specific antigen-presenting cells of the invention e.g., for minimally at least about 3 days, preferably at least about 5 days.

Whether the antigen-specific T lymphocytes are produced in contact with antigen-presenting cells in vitro or in vitro, the antigen-specific anergy induced by the antigen-presenting cells of the present invention reflects the inability of the antigen-specific lymphocytes to respond to subsequent restimulation with the specific antigen. In accordance with the present invention, these antigen-specific lymphocytes are also preferably characterised by production of IL-10 in an antigen-specific manner. IL-10 is a cytokine with potent immunosuppressive properties. IL-10 inhibits antigen-specific T lymphocyte proliferation at different levels. IL-10 inhibits the antigen-presenting and accessory cell function of professional antigen-presenting cells such as monocytes, dendritic cells and Langerhans cells by downregulation of the expression of MHC class II molecules and of the adhesion and co-stimulatory molecules ICAM-1 and B7.1 and B7.2 (reviewed in Interleukin 10, de Vries and de Waal Malefyt, eds., Landes Co, Austin Tex., 1995). IL-10 also inhibits IL-12 production by these cells. IL-12 promotes T lymphocyte activation and the differentiation of Th1 lymphocytes (D'Andrea, et al., 1993, J. Exp. Med. 178:1041-1048; Hsieh et al., 1993, Science 260:547-549). In addition, IL-10 directly inhibits T lymphocyte proliferation by inhibiting IL-2 gene transcription and IL-2 production by these cells (reviewed in Interleukin 10, de Vries and de Waal Malefyt, eds., Landes Co, Austin Tex., (1995)), and itself promotes antigen-presenting cells that induce regulatory T cells (U.S. Pat. No. 6,277,635) (Groux et al., 1996).

Thus, in a preferred embodiment, the presence of anergic T lymphocytes may be determined by assaying IL-10 production. IL-10s exhibit several biological activities which could form the basis of assays and units. See, e.g., Coligan (ed) Current Protocols in Immunology (Greene/Wiley, NY, 1989 and periodic supplements). In particular, IL-10s have property of inhibiting the synthesis of at least one cytokine in the group consisting of IFNγ, lymphotoxin, IL-2, IL-3, and GM-CSF in a population of T helper cells induced to synthesise one or more of these cytokines by exposure to antigen and antigen presenting cells. In this activity, the antigen-presenting cells are treated so that they are incapable of replication, but that their antigen processing machinery remains functional. This is conveniently accomplished by irradiating the antigen-presenting cells, e.g., with about 1500-3000 R (gamma or X-radiation) before mixing with the T cells. Preferably, though IL-10 is assayed by ELISA in cell supernatants, or by flow cytometric analysis of intracellular staining (O'Sullivan and Thomas, 2002; Rissoan et al., 1999).

Alternatively, cytokine inhibition may be assayed in primary or, preferably, secondary mixed lymphocyte reactions (MLR), in which case syngeneic antigen-presenting cells need not be used. MLRs are well known in the art, e.g., Bradley, pgs. 162-166, in Mishell, et al., eds. Selected Methods in Cellular Immunology (Freeman, San Francisco, 1980); and Battisto et al. (1987, Meth. in Enzymol. 150:83-91, Academic Press). Briefly, two populations of allogeneic lymphoid cells are mixed, one of the populations having been treated prior to mixing to prevent proliferation, e.g., by irradiation. Preferably, the cell populations are prepared at a concentration of about 2×10⁶ cells/mL in supplemented medium, e.g., RPMI 1640 with 10% foetal calf serum. For both controls and test cultures, mix 0.5 mL of each population for the assay. For a secondary MLR, the cells remaining after 7 days in the primary MLR are re-stimulated by freshly prepared, irradiated stimulator cells. The sample suspected of containing IL-10 may be added to the test cultures at the time of mixing, and both controls and test cultures may be assayed for cytokine production from 1 to 3 days after mixing.

The anergy provided herein involves either a much lowered proliferative responsiveness to antigen, e.g., less than about 50% response, usually less than about 40% response, more usually less than about 5-10% response or less, as compared to non-anergic cells. When stimulated with specific antigen, these anergic cells produce less than about 50% and more usually 5-10% or less interferon-γ than non-anergic T lymphocytes in response to antigen. In contrast, they produce no interleukin-4, but 50% more or greater, and more usually 2-5 fold more interleukin-10 than non-anergic T lymphocytes in response to antigen.

5. Cell Based Therapy or Prophylaxis

The antigen-specific antigen-presenting cells described in Section 3 and the anergic lymphocytes described in Section 4 can be administered to a patient, either by themselves or in combination, for modifying an immune response, especially for inducing a tolerogenic response including the induction of an anergic response, and the suppression of a future or existing immune response, to one or more cognate antigens. These cell based compositions are useful, therefore, for treating or preventing an unwanted immune response including, for example, transplant rejection, graft versus host disease, allergies, parasitic diseases, inflammatory diseases and autoimmune diseases. Examples of transplant rejection, which can be treated or prevented in accordance with the present invention, include rejections associated with transplantations bone marrow and of organs such as heart, liver, pancreas, kidney, lung, eye, skin etc. Examples of allergies include asthma, hayfever, food allergies, animal allergies, atopic dermatitis, rhinitis, allergies to insects, fish, latex allergies etc. Autoimmune diseases that can be treated or prevented by the present invention include, for example, psoriasis, systemic lupus erythematosus, myasthenia gravis, stiff-man syndrome, thyroiditis, Sydenham chorea, rheumatoid arthritis, diabetes and multiple sclerosis. Examples of inflammatory disease include Crohn's disease, colitis, chronic inflammatory eye diseases, chronic inflammatory lung diseases and chronic inflammatory liver diseases.

The cells of the invention can be introduced into a patient by any means (e.g., injection), which produces the desired modified immune response to an antigen or group of antigens. The cells may be derived from the patient (i.e., autologous cells) or from an individual or individuals who are MHC matched or mismatched (i.e., allogeneic) with the patient. Preferably, autologous cells are injected back into the patient from whom the source cells were obtained. The injection site may be subcutaneous, intraperitoneal, intramuscular, intradermal, or intravenous. The cells may be administered to a patient already suffering from the unwanted immune response or who is predisposed to the unwanted immune response in sufficient number to prevent or at least partially arrest the development, or to reduce or eliminate the onset of, that response. The number of cells injected into the patient in need of the treatment or prophylaxis may vary depending on inter alia, the antigen or antigens and size of the individual. This number may range for example between about 10³ and 10¹¹, and more preferably between about 10⁵ and 10⁷ cells (e.g., dendritic cells or T lymphocytes). Single or multiple administrations of the cells can be carried out with cell numbers and pattern being selected by the treating physician. The cells should be administered in a pharmaceutically acceptable carrier, which is non-toxic to the cells and the individual. Such carrier may be the growth medium in which the cells were grown, or any suitable buffering medium such as phosphate buffered saline. The cells may be administered alone or as an adjunct therapy in conjunction with other therapeutics known in the art for the treatment or prevention of unwanted immune responses for example but not limited to glucocorticoids, methotrexate, D-penicillamine, hydroxychloroquine, gold salts, sulfasalazine, TNFα or interleukin-1 inhibitors, and/or other forms of specific immunotherapy.

In order that the invention may be readily understood and put into practical effect, particular preferred embodiments will now be described by way of the following non-limiting examples.

### EXAMPLES

### Example 1

Inhibition of Myeloid Dc Differentiation and Antigen-Specific Suppression of Primed Immune Responses by Inhibition of Relb Function

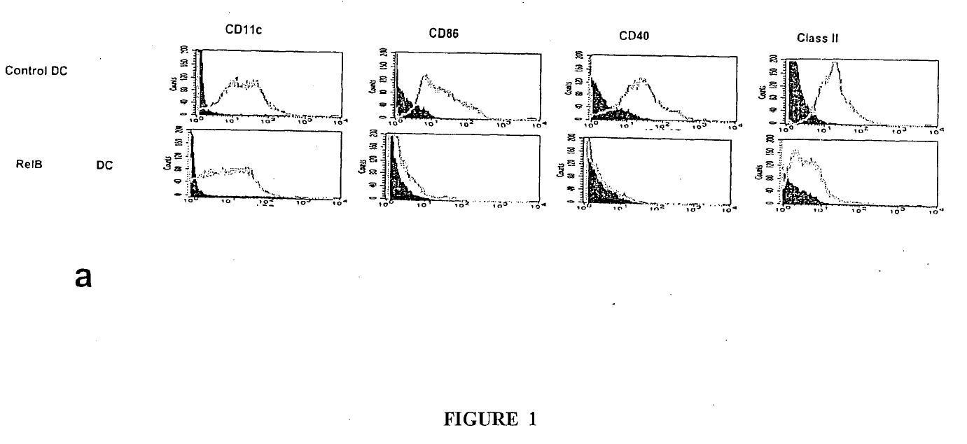

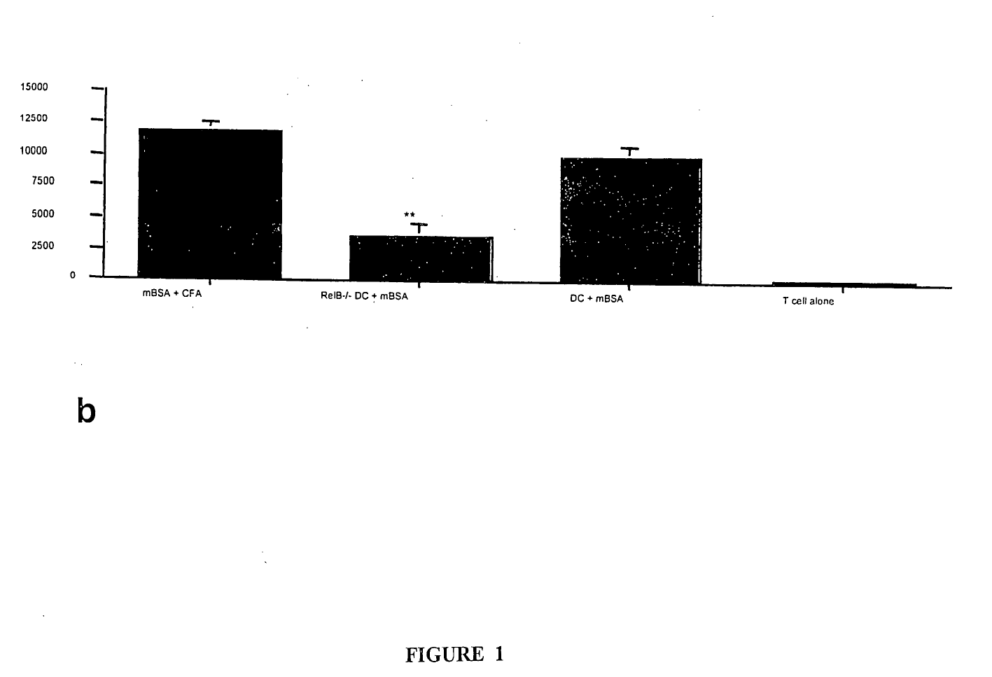

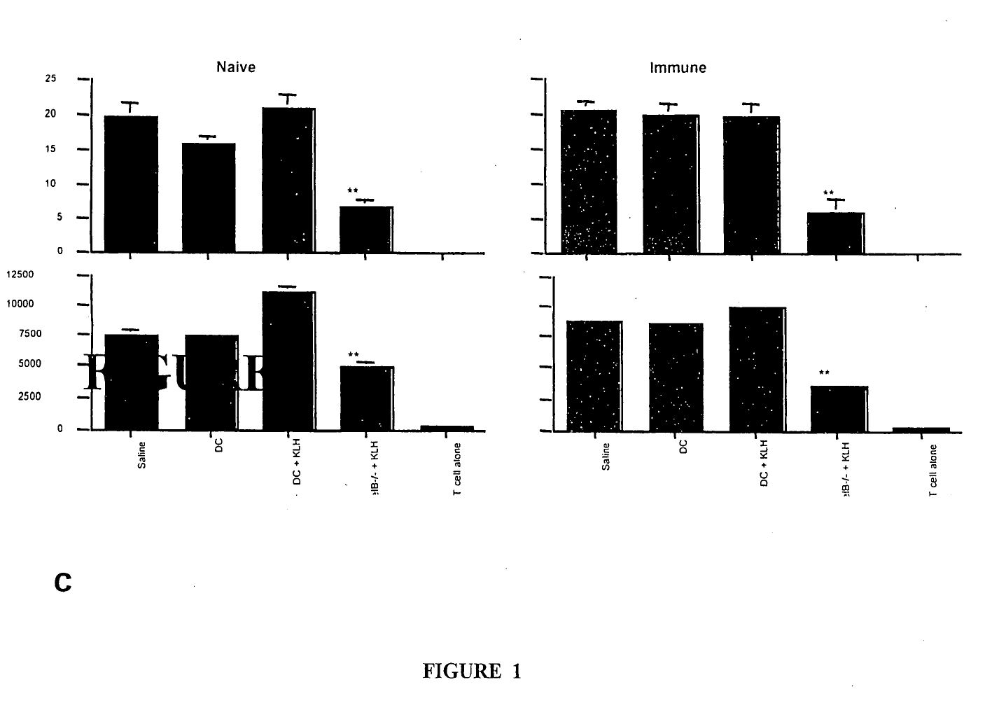

Translocation of the NFκB family members RelB and p50 from cytoplasm to nucleus is required for myeloid DC maturation (Burkly et al., 1995). To assess the relationship between RelB, differentiation, and tolerance induction by myeloid DCs, BMDCs were generated from homozygous H-2^{b} RelB^{-/-} or wild type H-2^{b} mice. RelB^{-/-} BMDCs did not express CD40, and expressed lower levels of MHC class 11 and CD86 than RelB^{+/+} BMDCs (FIG. 1a). The ability of s.c. adoptively-transferred mBSA-pulsed RelB^{-/-} BMDCs to prime an antigen-specific T cell proliferative response in naïve wild type H-2^{b} mice was reduced, compared with mBSA-pulsed RelB^{+/+} BMDCs (FIG. 1b). To test tolerance induction, wild type H-2^{b} mice were injected s.c. with 5×10⁵ KLH-pulsed DCs 7 days before or 7 days after priming with KLH in CFA. KLH-specific immunity was tested 5d after DC or KLH administration. Administration of DCs generated from RelB^{-/-} BM not only prevented priming of specific immunity by subsequent administration of KLH/CFA. but also suppressed a previously primed immune response when administered 7 days after immunisation with KLH/CFA (FIG. 1c). In contrast, no suppression of KLH specific immunity was observed following administration of KLH pulsed RelB^{+/+} DCs, whether these DCs were administered prior to or following immunisation. (FIG. 1c).

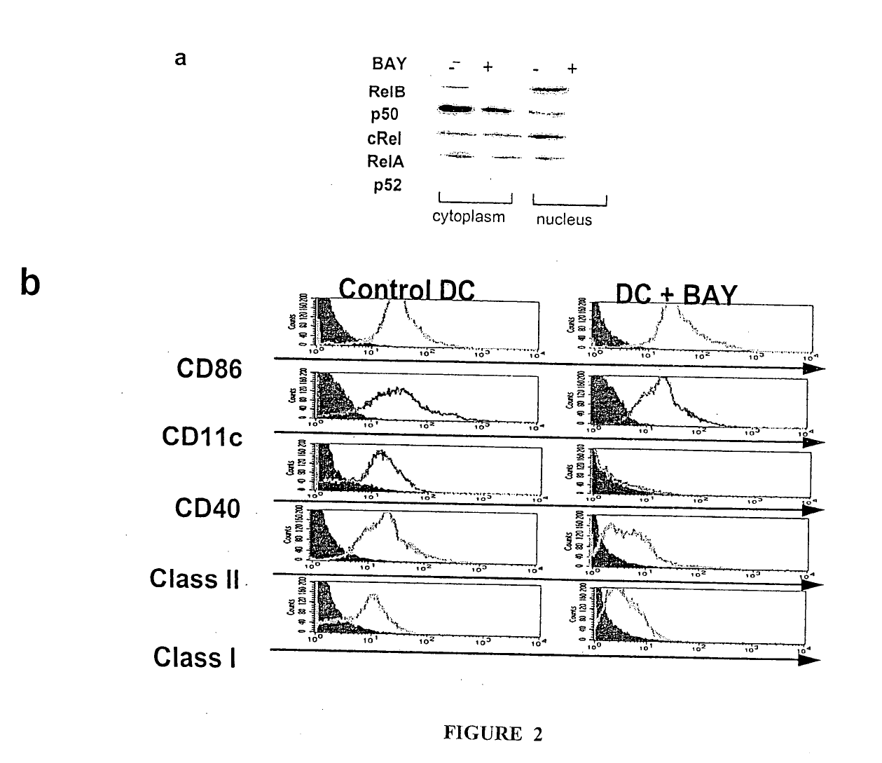

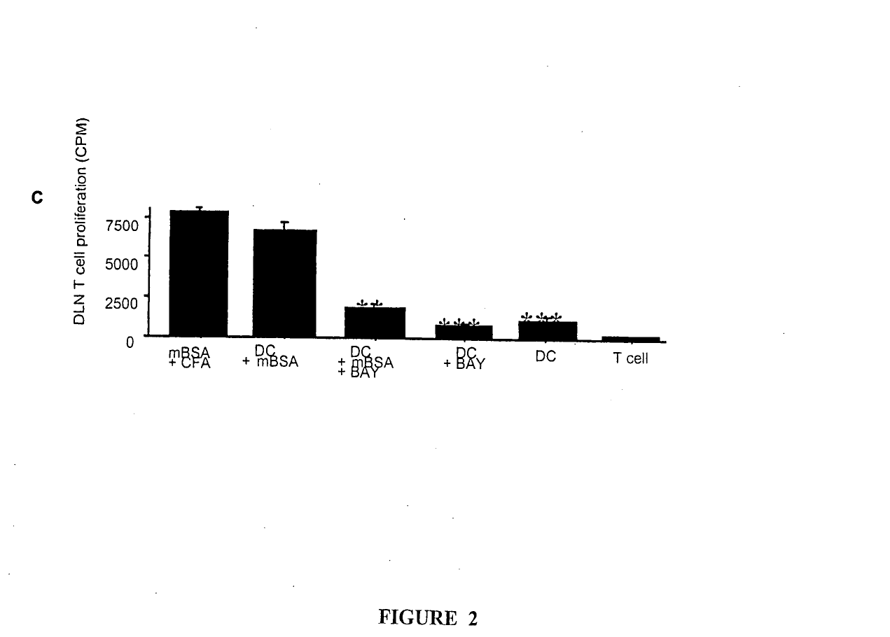

To independently assess the relationship between RelB nuclear translocation and tolerance induction, BMDCs were generated in the presence or absence of the compound BAY 11-7082 (BAY). BAY has been shown to block TNF-α-stimulated NF-κB translocation through inhibition of IκBα phosphorylation (Pierce et al., 1997). Murine BM precursors were incubated for 6 days with GM-CSF and IL-4 to produce BMDCs, in the presence or absence of BAY. All NF-κB subunits were demonstrable in BMDCs generated in the absence of BAY, and were present in both nuclear and cytoplasmic extracts (FIG. 2a). In contrast, BMDCs generated in the presence of BAY demonstrated NFκB subunit immunoreactivity in the cytoplasm but not the nucleus (FIG. 2a). BMDCs generated in the presence of BAY were similar in phenotype to RelB-deficient DCs, in that they lacked cell surface CD40 expression, and expressed reduced levels of MHC class I and class II (FIG. 2b). However, CD86 was expressed at higher levels in BAY-treated DCs than RelB-deficient DCs. BMDC populations generated in the presence or absence of BAY had a dendritic morphology, and expressed CD11b and low levels of F4/80, but no CD8α, CD3, CD19, Ly6-c, or CD45R (data not shown). The ability of adoptively-transferred BAY-treated DCs to prime an mBSA-specific T cell proliferative response in naïve syngeneic mice when pulsed with the antigen mBSA was reduced, compared with mBSA-pulsed untreated BMDCs, or immunisation with mBSA and CFA (FIG. 2c).

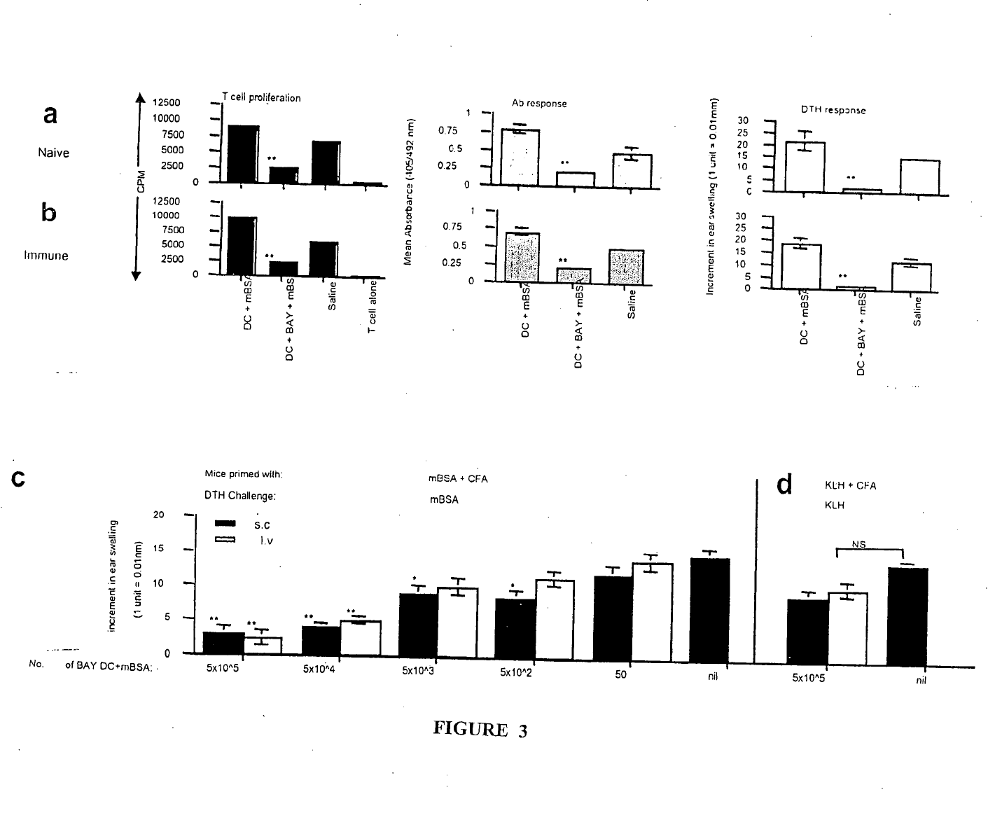

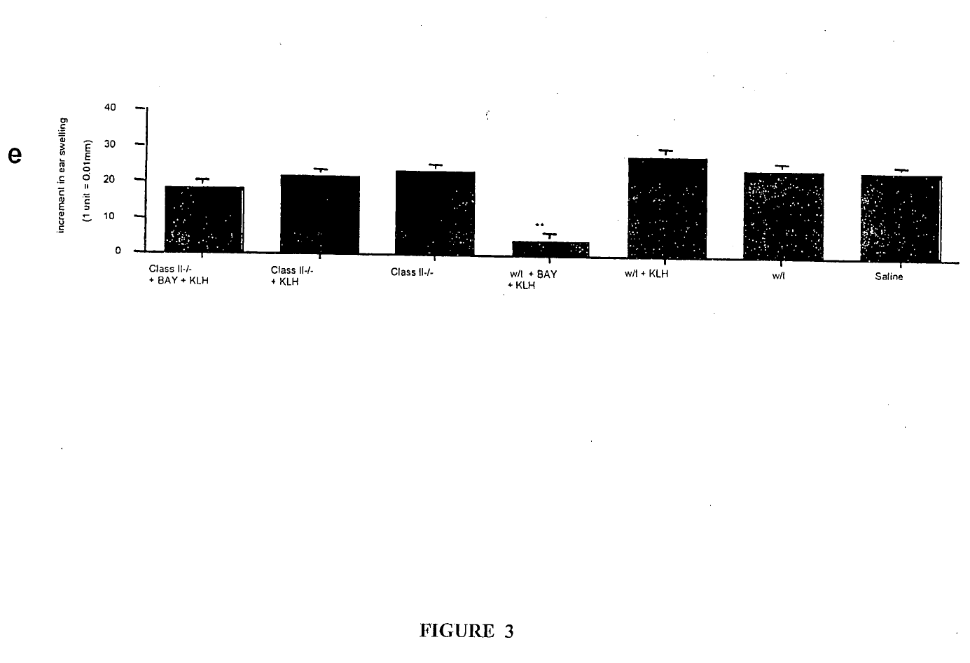

BAY-treated DCs were administered to animals 7 days before or 7 days after immunisation with mBSA in CFA. mBSA-specific immunity was tested 5 d after DC or mBSA administration. mBSA-pulsed BAY-treated DCs prevented priming and conferred suppression of mBSA-specific immunity when compared to mBSA-pulsed DCs that had not been exposed to BAY (FIG. 3). mBSA-specific T cell proliferation, Ab production and DTH responses were each suppressed after administration of antigen-exposed BAY-treated DCs. The data indicate that DCs in which RelB function is inhibited lack CD40, prevent subsequent priming of immunity, and suppress a previously primed immune response in vivo.

The antigen specificity of suppression, and the effect of dose and route of immunisation were tested by comparing the DTH responses to KLH and to mBSA after administration of varying doses of mBSA-pulsed BAY-treated DCs given i.v. or s.c. Mice injected with mBSA-pulsed BAY-treated DCs were tolerant to subsequent priming with mBSA in CFA, in a route-independent fashion, and greater numbers of DCs were more effective at inducing tolerance (FIG. 3c). There was no reduction in KLH DTH responses in mice pre-injected with 5×10⁵ mBSA-pulsed BAY-treated DCs and subsequently primed with KLH in CFA (FIG. 3d). The data indicate that the tolerance induced by BAY-treated DCs is DC dose dependent and specific for the antigen to which the DCs have been exposed. The lack of suppression of the KLH DTH response also excludes carry over of non-specific suppressive effects by residual soluble inhibitor to draining LN lymphocytes.

While injected DCs are likely to present antigen directly to T cells in draining LN, it was possible that antigen-exposed injected DCs could be cross-presented by host DCs in recipient mice. To address this, 5×10⁵ KLH-pulsed H-2^{b} wild type or MHC class II^{-/-} BMDCs generated in the presence or absence of BAY were administered to wild type mice 7 days after immunisation with KLH in CFA. KLH-specific immunity was tested 5 days after DC administration. KLH-pulsed BAY-treated wild type DCs conferred suppression of KLH-specific immunity when compared to KLH-pulsed BAY-treated MHC class II^{-/-} DCs or KLH-pulsed wild-type DCs that had not been exposed to BAY. Neither KLH-specific T cell proliferation (not shown), nor DTH responses were each suppressed after administration of antigen-exposed BAY-treated MHC class II^{-/-} DCs (FIG. 3e). The data indicate that MHC class II expression by the injected DCs is necessary for subsequent suppression, thereby providing evidence that injected BAY-treated antigen-exposed DCs are not cross-presented by recipient DCs.

### Example 2

Suppression of Primed Immune Responses by DCS Correlates with their RelB Nuclear Binding Activity and CD40 Expression

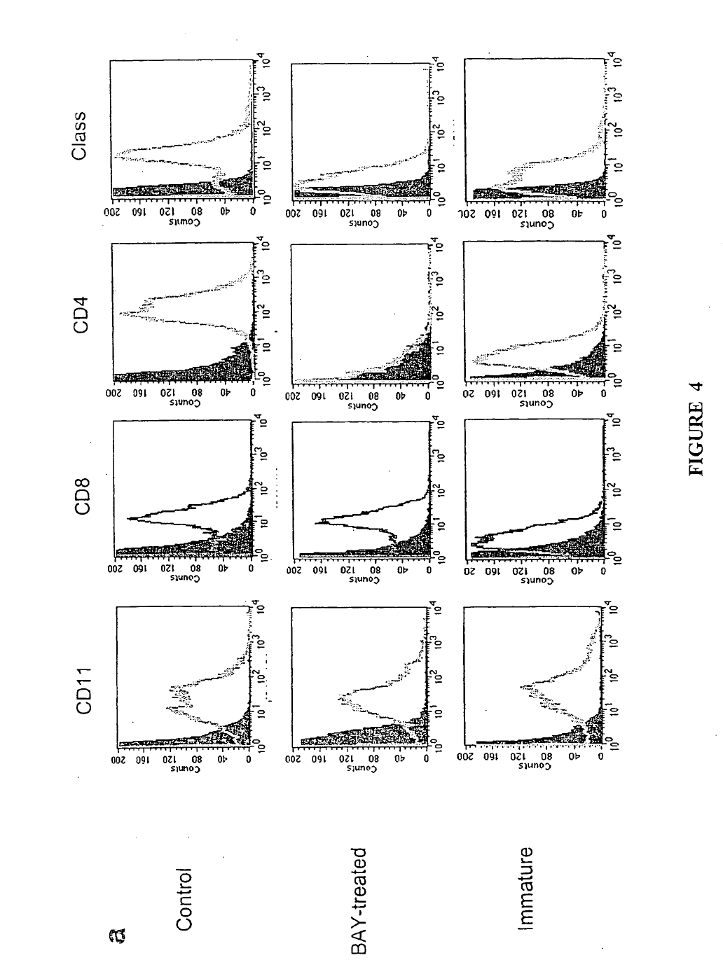

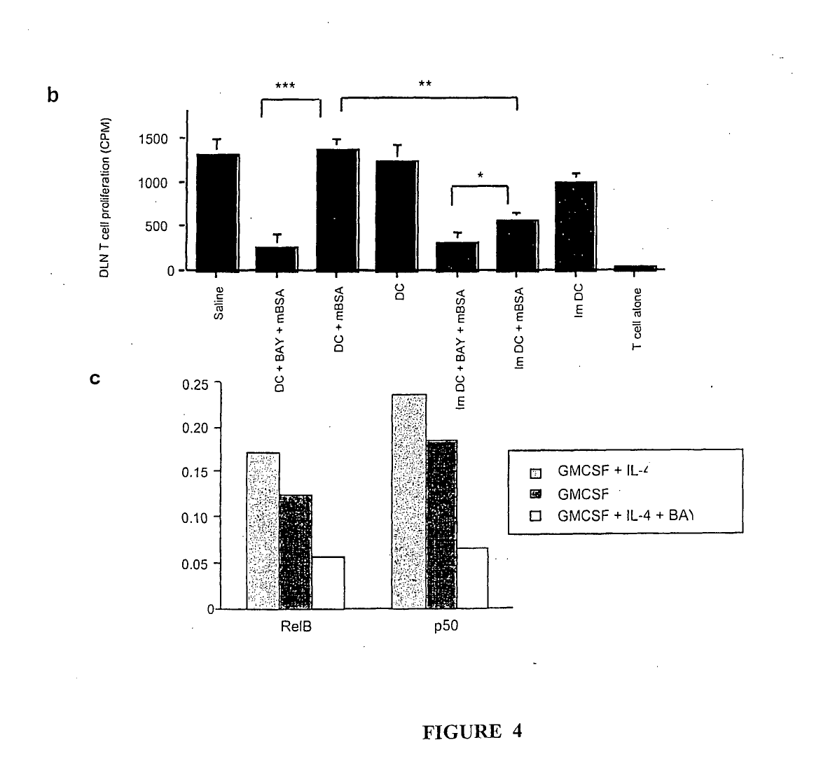

Previously, immature BM or monocyte-derived DCs have been shown to regulate immune responses (Dhodapkar et al., 2001; Jonuleit et al., 2000). Since DCs in which RelB nuclear translocation is inhibited suppressed a primed immune response, the capacity of DCs prepared ex vivo to suppress immunity was correlated with RelB activity in nuclear extracts. BMDCs were generated in serum-containing medium either in GM-CSF and IL-4 (control DCs), in GM-CSF alone (“immature DCs”), (Lutz et al., 2000) or in BAY, GM-CSF and L-4, then pulsed with KLH and injected s.c. into mice primed 7 days previously with KLH and CFA. Immature DCs expressed lower levels of CD86, CD40, and class II than control DCs. By contrast, BAY-treated DCs expressed higher levels of CD86 and reduced CD40 compared with immature DCs (FIG. 4a). Suppression by DCs of KLH-specific draining LN T cell responses correlated with the binding capacity of RelB and p50 in nuclear extracts to an NF-κB consensus oligonucleotide, and with CD40 expression (FIG. 4). Immunocytochemical staining of DC populations showed that the proportion of DCs that had translocated RelB to the nucleus within each population correlated with total RelB and p50 binding capacity (data not shown).

### Example 3

CD40 Deficiency is Sufficient to Confer Suppression of Immunity by DCs.

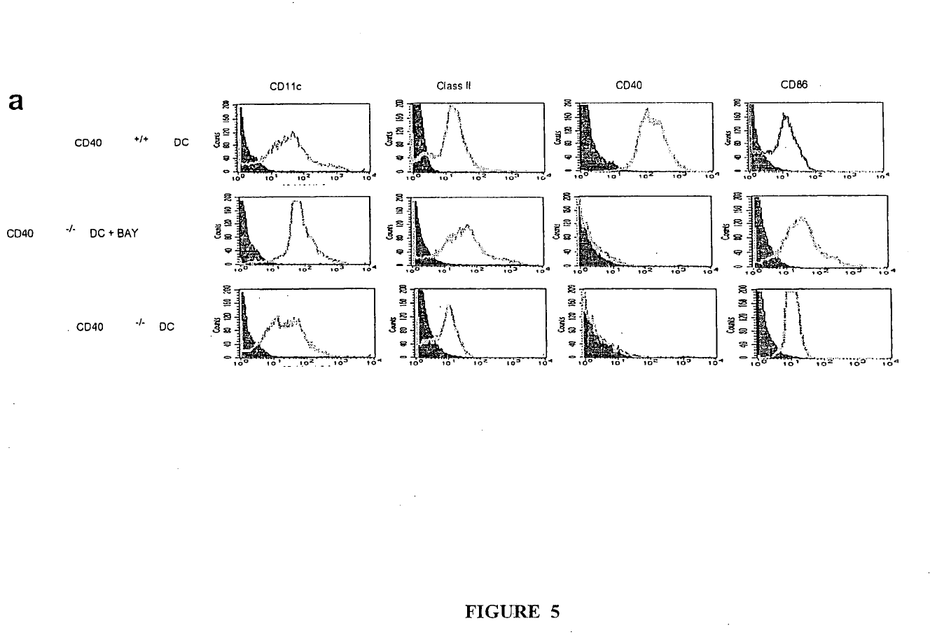

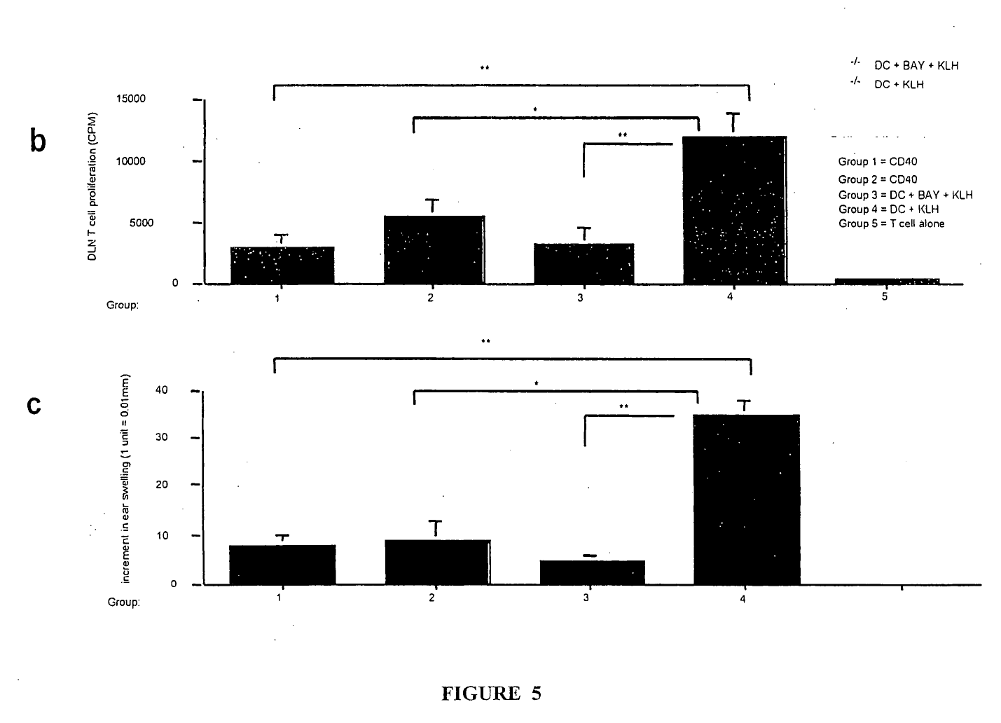

Since RelB deficient and BAY-treated BMDCs lacked cell surface CD40, and suppression of immunity by DCs correlated with CD40 expression, we determined whether lack of CD40 expression by antigen-exposed BMDCs was sufficient for suppression of previously primed immunity. DCs generated from CD40^{-/-} BM in the presence or absence of BAY were similar in phenotype to RelB-deficient DCs, except for higher cell surface CD86 expression (FIG. 5a). DCs generated from BM of H-2^{d} CD40^{-/-} or CD40^{+/+} mice in the presence or absence of BAY were pulsed with KLH and administered s.c. to wild type H-2^{d} mice 7 days after priming with KLH in CFA. Administration of DCs generated from CD40^{-/-} BM conferred equivalent suppression of a previously primed immune response to DCs generated from either CD40^{-/-} or CD40^{+/+} BM in the presence of BAY (FIGS. 5b, c).

### Example 4

In the Absence of RelB or CD40, DCs Induce CD4⁺ Regulatory T Cells that Confer “Infections” Tolerance

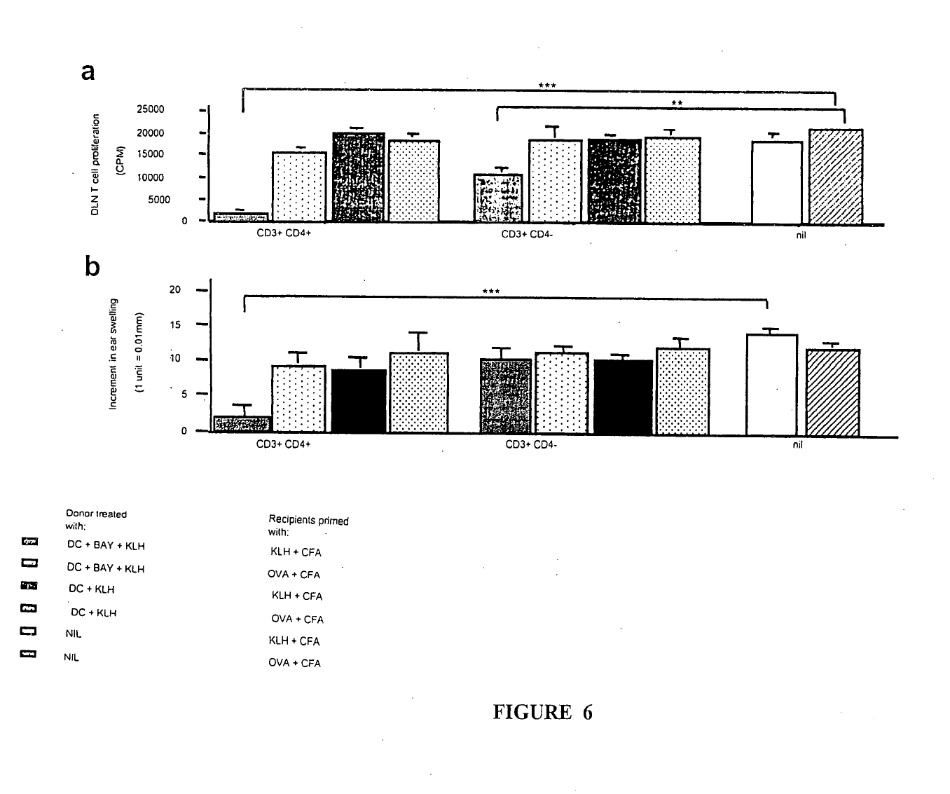

The systemic nature of the conferred suppression suggested that in the absence of CD40 or of RelB activity, DCs might induce Treg in the recipient animal. This possibility was examined in two ways. First, we tested whether induced Treg could transfer tolerance to naïve or primed recipient animals (“infectious tolerance”) (Cobbold and Waldmann, 1998). 5×10⁵ flow cytometrically sorted CD4⁺ or CD8⁺ T cells derived from spleens of mice injected s.c. with KLH-pulsed BAY-treated BMDCs were transferred to syngeneic recipients primed 9 days previously with either KLH or ovalbumin and CFA. Antigen-specific T cell responses were determined 7 days after T cell transfer. Adoptive transfer of CD4⁺ T cells derived from mice previously treated with KLH-pulsed BAY-treated BMDCs strongly suppressed the subsequent KLH-specific T cell proliferative responses in recipient mice, when compared with CD4⁺ or CD4⁻ T cells from mice treated with KLH-pulsed untreated BMDCs (FIGS. 6a, b). Suppression by CD4⁻ T cells derived from mice previously treated with KLH-pulsed BAY-treated BMDCs was modest or absent over a number of experiments. The OVA-specific T cell proliferative response in the recipient mice was unaffected by T cell transfers. Since these cells were identically sorted, it is unlikely that the labelling mAb themselves induced suppressive capacity by the T cells.

Second, cytokine production by CD4⁺ T cells in LN draining the site of antigen-exposed DC immunisation was compared ex vivo. When compared with the CD4⁺ T cells in LN draining the site of antigen pulsed RelB^{+/+} DCs, a greater proportion of CD4⁺ T cells in LN draining the site of injection of either antigen-pulsed RelB^{-/-} DCs, BAY-treated DCs or CD40^{-/-} DCs produced IL-10 in response to the mitogen PMA (Table 1). A greater proportion of T cells primed by KLH-pulsed BAY DCs also produced IL-10 in response to KLH but not to ovalbumin in vitro. In contrast, a greater proportion of T cells primed by KLH-pulsed untreated DCs produced IFN-γ in response to KLH but not to ovalbumin in vitro. IL-4 production was minimal in all draining LNs (data not shown). Taken together, the data indicate that DCs lacking CD40 expression or RelB function induce the differentiation of CD4⁺ Treg that are capable of producing IL-10 in an antigen-specific manner.

To determine whether the IL-10 produced by the CD4⁺ Treg was responsible for the observed suppression of immunity, 5×10⁵ DCs generated from BM of H-2^{b} wild type mice in the presence or absence of BAY were pulsed with KLH and administered s.c. to naïve IL-10^{+/+} or IL-10^{-/-} H-2^{b} mice. CD4⁺ T cells were magnetically sorted by negative selection from recipient spleens 7 days later and 2.5×10⁵ cells were transferred to wild type mice primed 7 days previously with KLH in CFA. Adoptive transfer of CD4⁺ T cells derived from IL-10^{+/+} mice previously treated with KLH-pulsed BAY-treated BMDCs strongly suppressed the subsequent KLH-specific DTH and T cell proliferative responses (not shown) in recipient mice, when compared with CD4⁺ derived from IL-10^{-/-} mice treated with KLH-pulsed BAY treated BMDCs (FIG. 6c). The data indicate that antigen-exposed DCs in which RelB function is inhibited induce a population of antigen-specific CD4⁺ regulatory T cells that regulate immune responses in an IL-10-dependent manner.

### Example 5

Modification of Human DC Differentiated from Monocyte Precursors in the Presence of GM-CSF and IL-4.

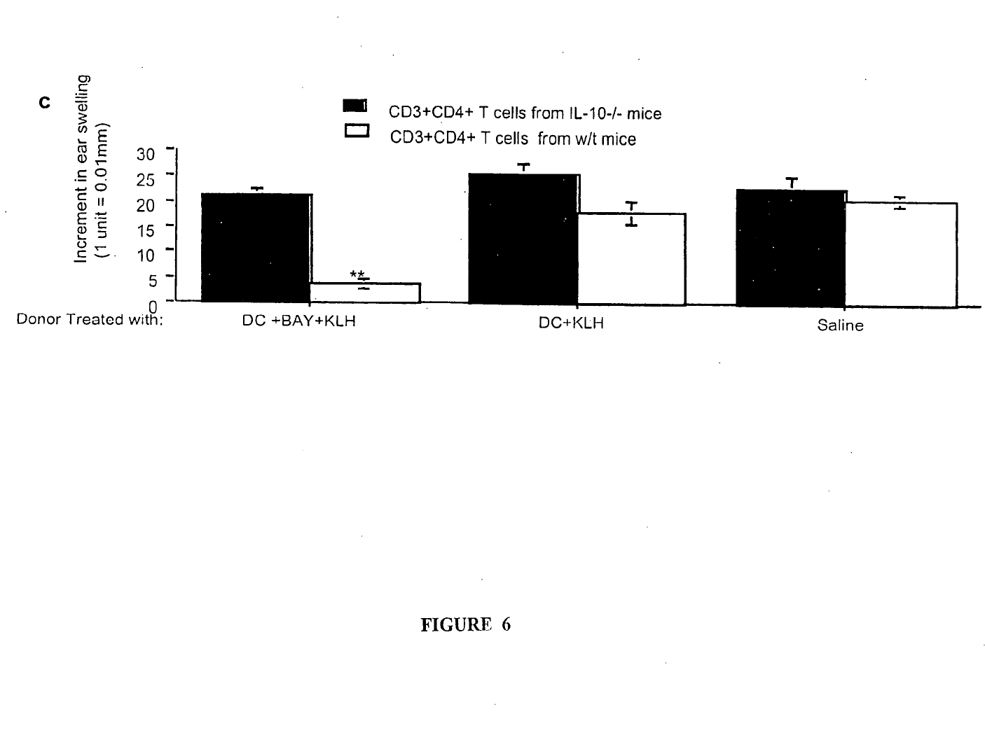

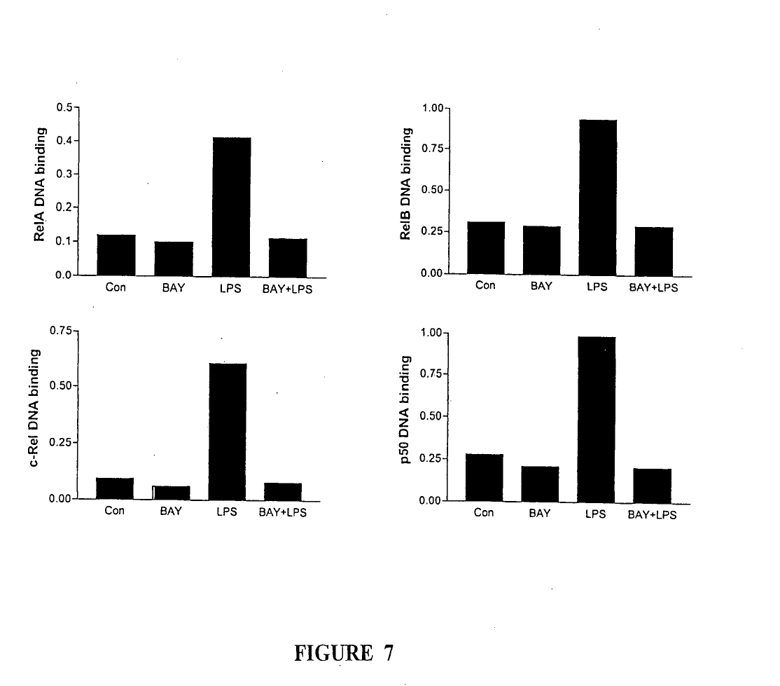

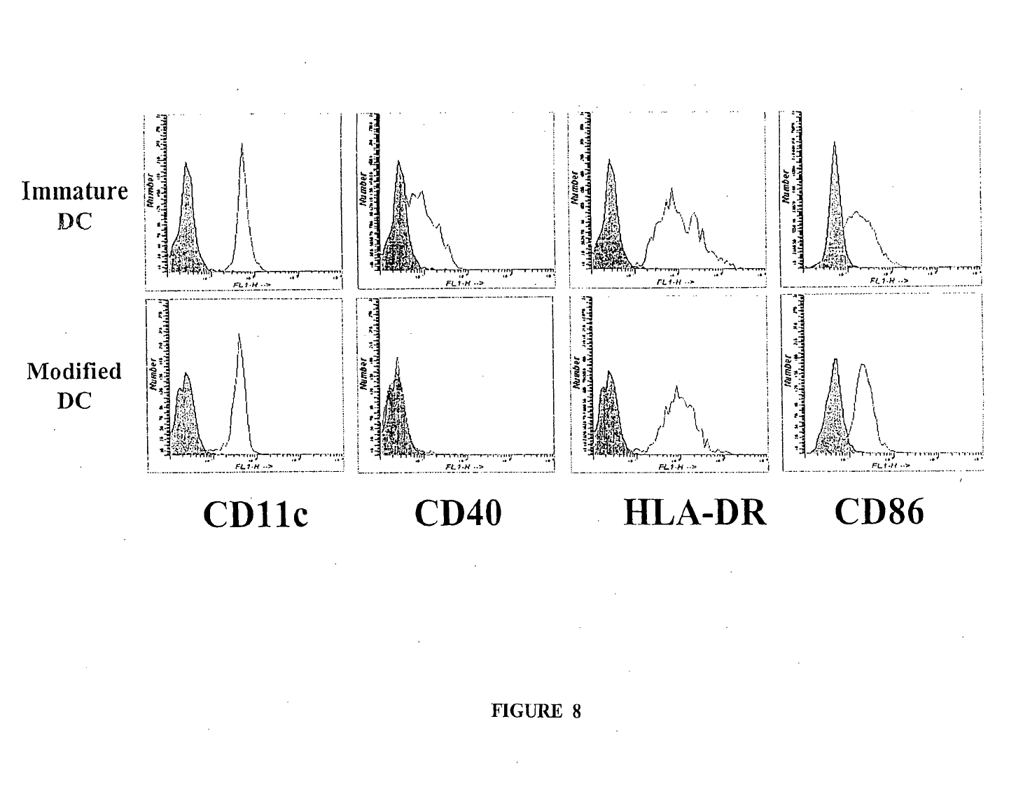

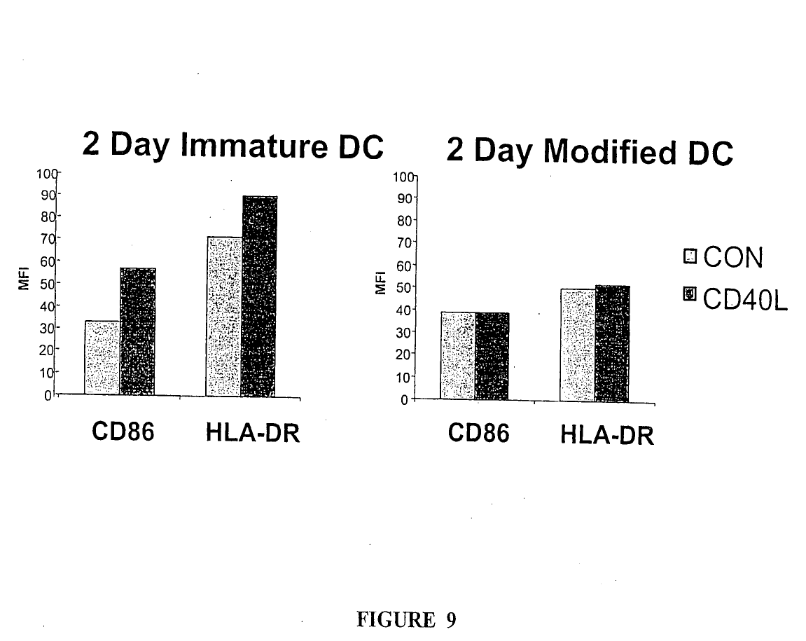

Human PB monocytes were purified from PBMC by immunomagnetic selection using anti-CD14. They were incubated for 48 h in the presence of 800 U/mL each GM-CSF and IL-4 in RPMI and 10% FCS in the presence or absence of 8 μM BAY 11-7082. Nuclear extracts were prepared and RelB DNA binding was assessed using ELISA, as previously described (O'Sullivan and Thomas, 2002). FIG. 7 shows that DC differentiated in the presence of BAY lacked RelB nuclear DNA binding activity, in contrast to those DC differentiated in the absence of BAY. FIG. 8 demonstrates the phenotype of the BAY-modified DC in comparison with immature DC differentiated in the absence of BAY. Modified DC lacked CD40 expression and expressed somewhat higher levels of CD86 than immature DC. In keeping with the lack of CD40 expression, modified DC were unresponsive to 24 h incubation with 500 ng/mL soluble CD40 ligand trimer (sCD40L, Immunex, Seattle Wash.). As shown in FIG. 9, by flow cytometry, the mean fluorescence intensity of CD86 and HLA-DR expression increased in immature DC in response to sCD40L but did not change in BAY-modified DC. The data indicate that BAY-modified DC neither express CD40 nor respond to CD40-ligation.

### Example 6

Effects of Modified DC on T Cell Function In Vitro.

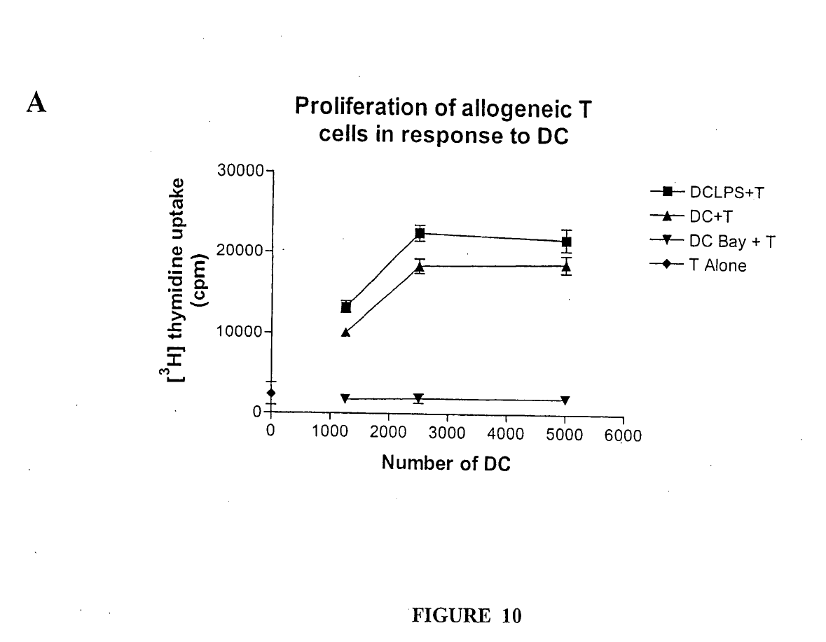

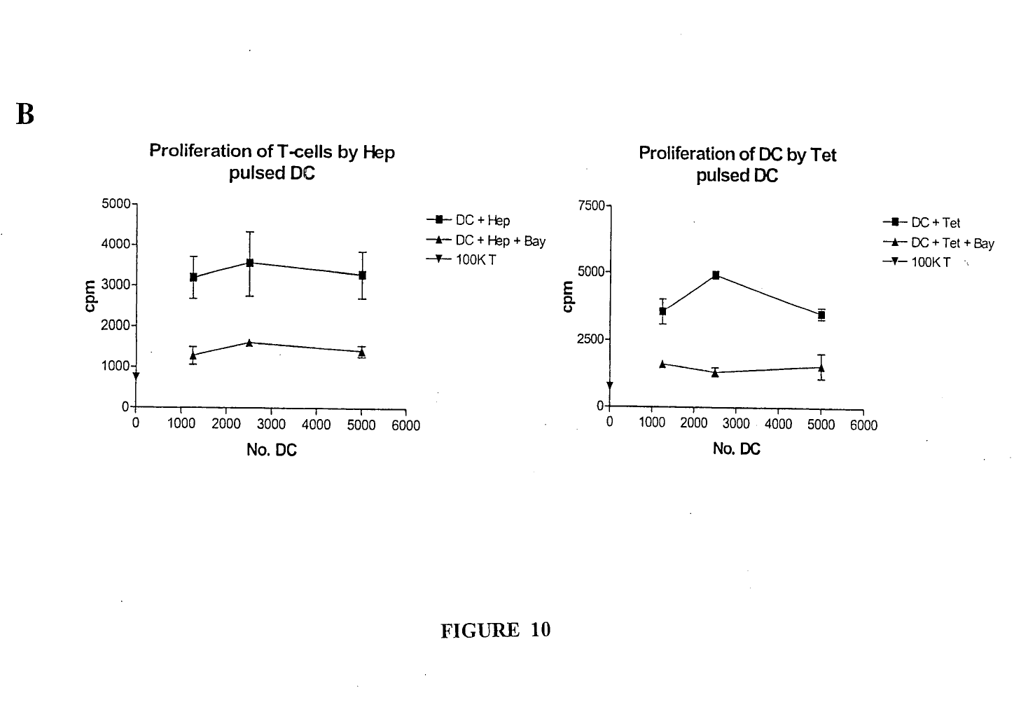

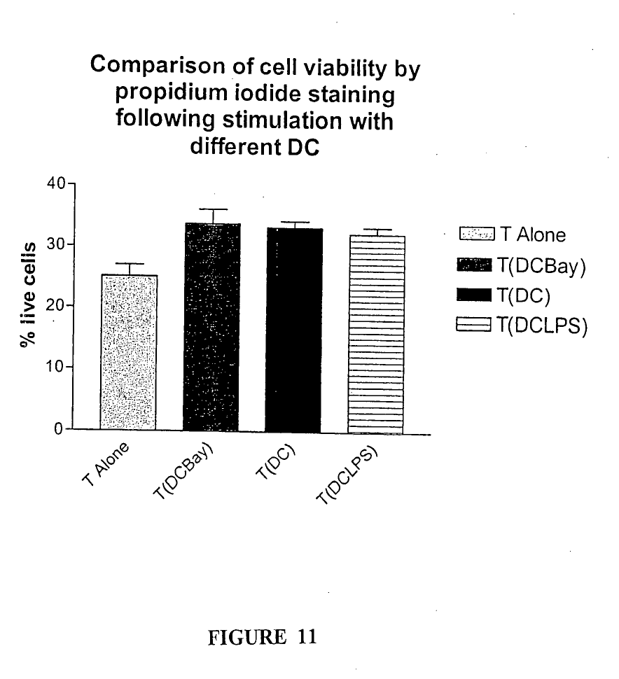

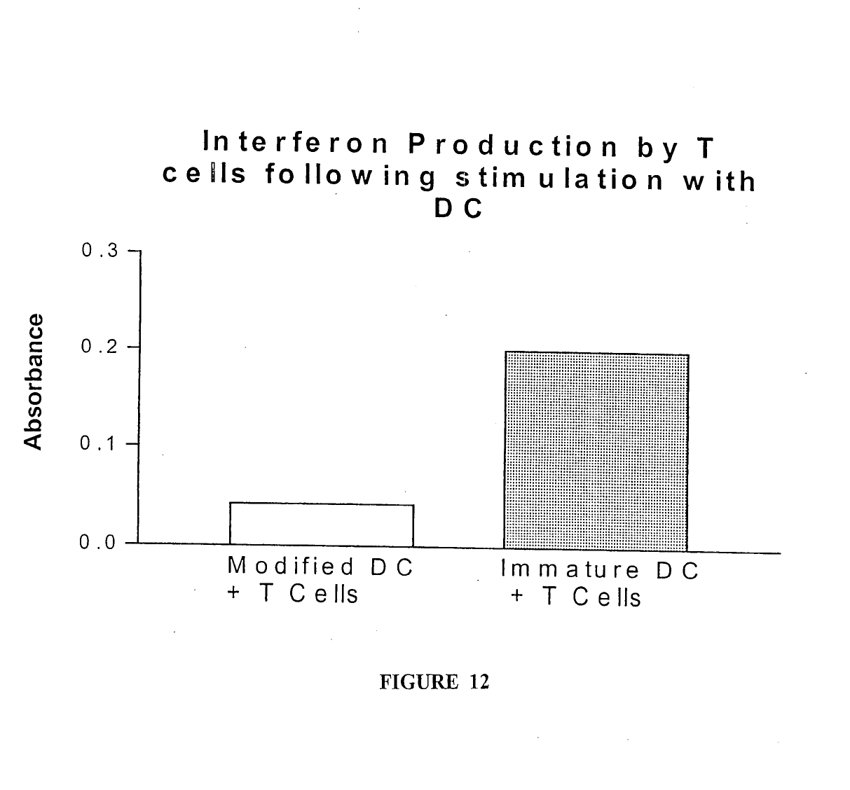

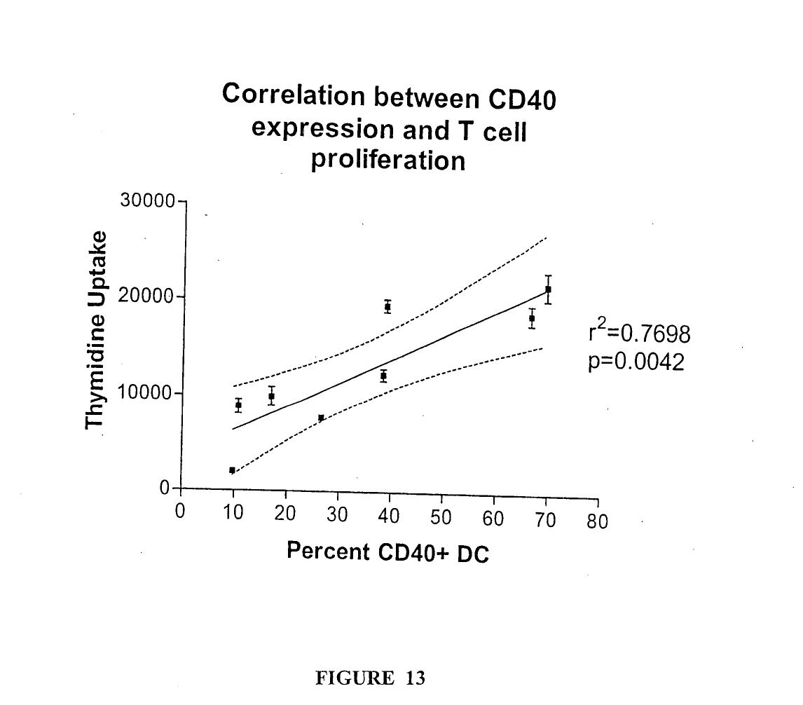

The next series of experiments used DC differentiated in the presence of BAY as described above as APC, to determine the capacity of T cells to respond to antigen-presented by those DC in comparison with either immature DC, or mature DC. Mature DC were generated from monocytes in the presence of GM-CSF and IL-4 as for immature DC, followed by addition of 100 ng/mL lipopolysaccharide for the final 24 h of culture. All DC were washed three times before addition to resting T cells. Resting T cells were purified from PBMC by sheep erythrocyte rosetting, followed by depletion of B cells, NK cells and APC by immunomagnetic separation. As shown in FIGS. 10 and 11, resting T cells made no proliferative response when stimulated with BAY-modified DC whether allogeneic, or autologous and loaded with the exogenous antigens, tetanus toxoid or hepatitis B surface antigen. In contrast, T cells proliferated in response to either immature or mature DC. FIG. 12 demonstrates that this lack of proliferation was not due to T cell death as the viability of T cells at the end of a 6 day co-culture with BAY-modified or unmodified DC was not reduced. Viability was assessed by flow cytometry using propidium iodide staining. In data not shown, transfer of the supernatant from the DC differentiation cultures did not inhibit T cell proliferation in mixed lymphocyte cultures, indicating that the lack of T cell proliferation could not be explained by carry-over of BAY 11-7082 into the MLR, leading directly to suppression of T cells. In keeping with the lack of T cell proliferation, no IFN-γ could be detected in the supernatant of allogeneic MLR in which BAY-modified DC acted as stimulators. In contrast, T cells stimulated by immature or mature DC proliferated in a DC concentration-dependent manner. To determine the relationship between CD40 expression by DC and T cell proliferation, DC were differentiated from monocytes in the presence of varying concentrations of BAY, then their expression of CD40 was analysed by flow cytometry, and the DC were used as stimulators in allogeneic MLR. As shown in FIG. 13, the T cell proliferative response was closely correlated with the % DC that express cell surface CD40. The data indicate that following incubation with BAY-modified DC, T cells fail to proliferate and to secrete detectable IFN-γ. This effect on T cells appears to relate to the level of CD40 expressed by the DC.

### Example 7

Suppression of Antigen-Induced Arthritis

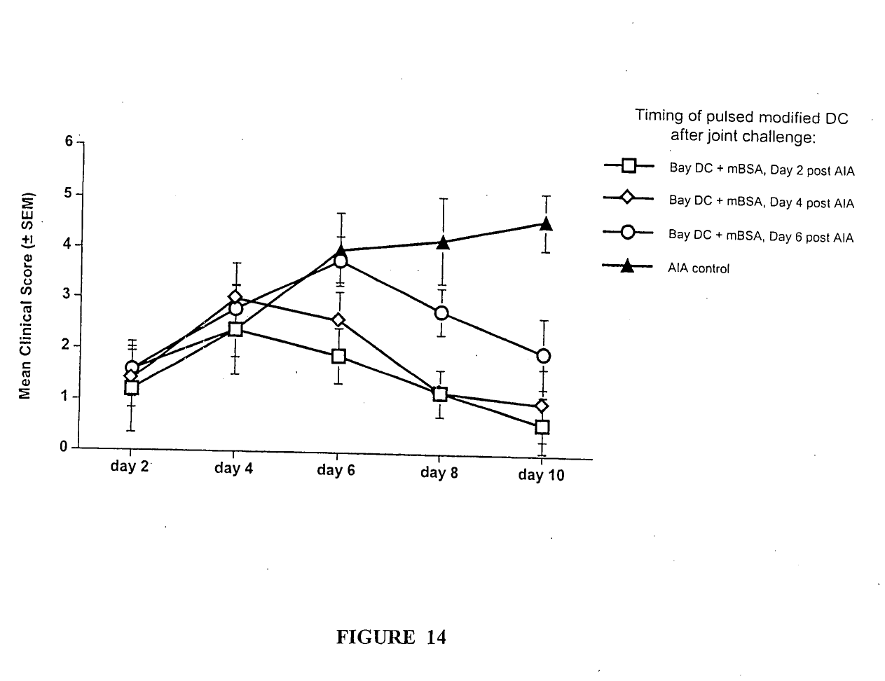

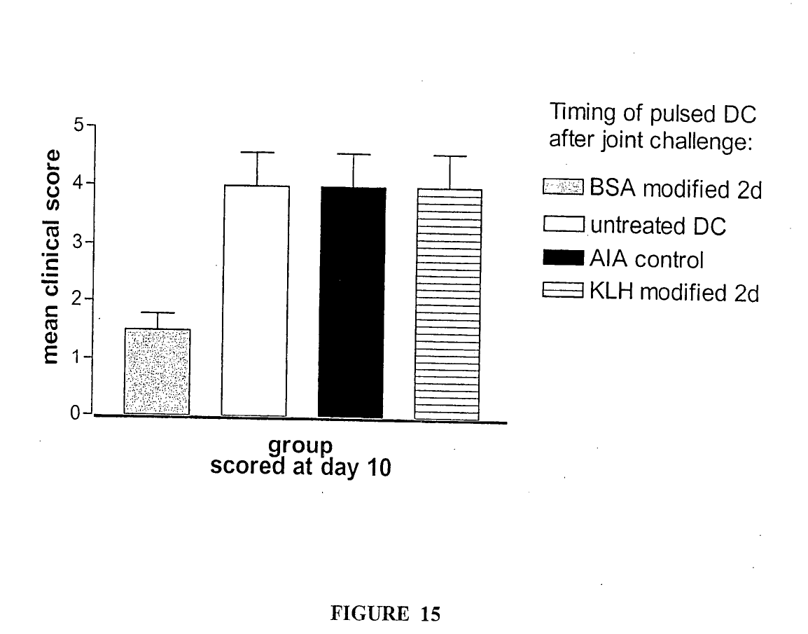

The above Examples demonstrate that antigen-exposed dendritic cells (DC), in which RelB function is inhibited (modified DC), lack cell surface CD40, prevent priming of immunity, and suppress previously primed immune responses. They also show that regulatory CD4⁺ T cells induced by the DC transferred antigen-specific “infectious” tolerance to primed recipients in an interleukin-10 dependent fashion. In this example, the present inventors show that modified DC (generated in the presence of BAY11-7082), which are exposed to arthritogenic antigen, are able to suppress arthritis even after expression of clinical disease. Specifically, a model of antigen-induced arthritis was employed in which mice were primed to mBSA. Eight days later, arthritis was induced in one knee joint by the injection of mBSA. The other knee joint was used as a non-arthritic control, and was injected only with saline. Clinical arthritis developed over 5 days and was scored semi-quantitatively, based on the amount of joint swelling. However, when mBSA-exposed modified DC were injected subcutaneously at various times after joint injection these DC suppressed clinical arthritis even after disease has fully established at day 6 (FIG. 14). This suppression was shown to be antigen-specific and to require exposure to BAY11-7082, since neither KLH-exposed modified DC, nor untreated DC induce disease suppression. These results demonstrate, therefore, that modified DC injected s.c. can suppress established arthritis in an antigen-specific manner.

### Example 8

BMDC Preparation: Ficoll-Paque Method Using Complete RPMI-1640

The following protocol is used to prepare high quality BMDC:
1. Extract bones from 5-10 mice (femur and tibia). Place in a small volume of medium (approx 10 mL) (complete RPMI-1640) and keep on ice.
2. Conduct all following procedures in a sterile laminar flow cabinet:
3. Immediately flush bone marrow using 21G needle and medium into a petri dish. Transfer cell suspension to a 50 mL tube.
4. Resuspend clumps of bone marrow thoroughly (using a mixing cannula and 10 mL syringe).
5. Filter through 70 μm cell strainer to remove debris.
6. Centrifuge at 1500 rpm 410 g/10° C./5 minutes. Discard supernatant and loosen pellet by flicking.
7. Resuspend pellet in 5 mL medium and transfer to 15 mL tube.
8. Underlay with 10 mL Ficoll-Paque avoiding bubbles. Centrifuge at 1200 rpm (390 g)/25° C./30 minutes with the BRAKE OFF.
9. Harvest buffy coat into a 50 mL Falcon tube. Wash 2× with medium (count cells during last wash).
10. Resuspend cells in 500 μL RPMI/1% FCS for every 5 mice BM. Adjust volume according to number of mice. Add a 1:500 dilution of each of the following purchased purified antibodies anti-Thy1.1, anti-B220, anti-IA/IE, 2.4G2 (anti-CD16/CD32) and 1:1000 anti-Gr1^{#}.
11. Incubate at room temperature for 10 minutes.
12. Add 45 mL medium to wash off excess antibody. Centrifuge at 1500 rpm/10° C./5 minutes.
13. Resuspend cells in 500 μL 1% FCS (HI)/PBS.
14. Add 0.5 μL MACS beads (Goat α Rat) for every 10⁶ cells/depleted. Incubate 4° C./15 minutes. Do not wash cells after this step.
15. Deplete cells using MACS column. Count cells.
16. Wash twice with medium.
17. Resuspend cells at 3-5×10⁶ cells/mL in medium containing 10 ng/mL mGM-CSF (e.g., add 10 μL/mL of 1 μg/mL stock) and 10 ng/mL mIL-4 with (tolerogenic DC) or without (control DC) 5 μM* BAY 11-7082.
18. Plate out in 24 well plates (1 mL/well). Incubate at 37° C.
19. Refresh medium, cytokines and BAY every 2 days (carefully remove 500 μL/well and replace with 500 μL/well fresh medium containing total cytokine concentrations for each well).
20. DC are ready for use between days 6 and 8. Approximate yields:

Bones from 12 mice aged 6-8 weeks or 8-10 older mice yield 10×10⁶ DC.

*Quality Control Issues: NFκB Inhibitor (BAY 11-7082)

50 μM STOCK: 10 mg in vial
Dissolve to 50 mM by adding 965 μL of DMSO

1 mM WORKING: Dilute to 1 mM in medium (1:50 dilution)
Final concentration of 5 μM 1:200 dilution or 5 μL per 1 mL of medium. Make 100 μL aliquots and store at −70° C. The potency is reduced by freeze-thawing multiple times.

SOURCE: BAY 11-7082 (CAT# EI-278)
Biomol Research Laboratories
5100 Campus Drive
Plymouth Meeting, Pa. 19462-1123
USA

According to the inventors' experience, every new batch of BAY needs to be titrated. The final concentration for mouse BMDC cultures may range between 2 and 15 μM. Since tolerance induction correlates precisely with CD40 expression by the DC, varying concentrations of BAY are added to BM DC cultures. At day 6-8, yield is check and viability test using PI and CD40 expression by FACS. The minimum concentration that completely suppresses CD40 expression is chosen. Higher concentrations usually are more toxic and affect yield and viability.

CD40 expression by murine BMDC generated using this protocol is relatively low. The present inventors have found that CD40 expression is best quantitated using a biotinylated primary (e.g., from Pharmingen) followed by streptavidin-fluorochrome conjugate. Certain directly labelled anti-CD40 mAb barely detect CD40 expression by murine BMDC (e.g., from Santa Cruz). If difficulty is experienced in this regard, the BMDC and BAY-treated BMDC can be stimulated overnight with 100 ng/ml LPS. Only BMDC will upregulate CD40 expression.

Tolerance Induction by BAY DC:

Viable DC that lack CD40 expression reliably induce tolerance. This can be tested by s.c. injection of 0.5×10⁶ OVA or KLH pulsed BAY DC, priming with OVA in CFA 7 days later, and checking OVA-specific DTH 5-7 days later.

# Alternative Method with No Depletion of BM Cells Before Culture:

A novel bulk-culture method for generating mature dendritic cells from mouse bone marrow cells has recently been described by Son et al., 2002, J Immunol Methods 262(1-2):145-57. Using this method, cells are ready for harvest around day 8-10. No enrichment is required with metrizamide gradients. However, it should be noted that when using this method, CD40 expression by BMDC not treated with BAY is lower than by the depletion method above.

### Example 9

Comparison of BAY DC with TNFα Neutralization and Treatment of Disease Flare.

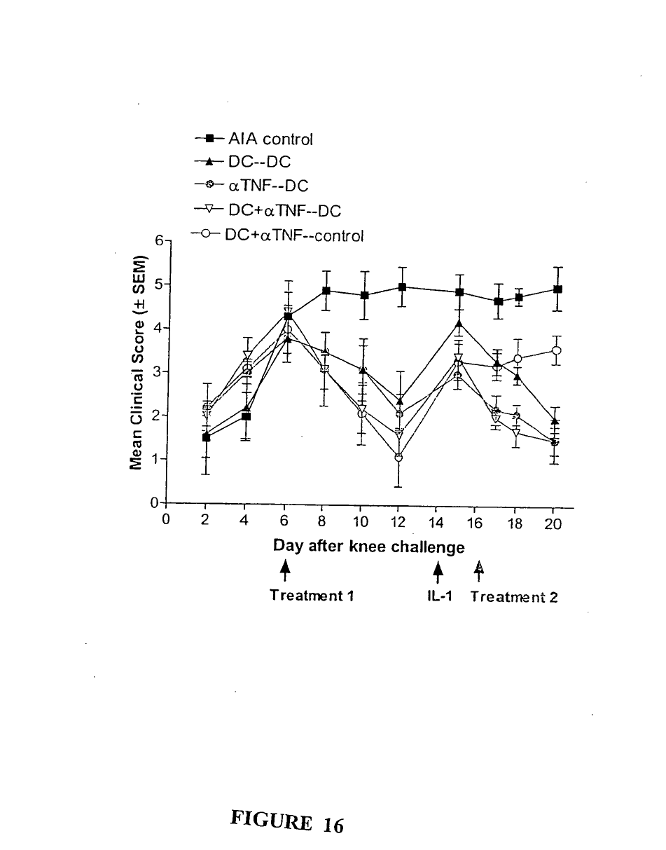

To test the efficiency of BAY DC in suppression of antigen-induced arthritis, the inventors compared them to neutralizing anti-tumor necrosis factor (TNF)-α monoclonal antibodies. Mice with AIA were treated with either 5×10⁵ mBSA-exposed BAY DC, anti-TNFα, both 5×10⁵ mBSA-exposed BAY DC and anti-TNFα, or no treatment 6 days after induction of arthritis by injection of the knee joints with mBSA antigen. Arthritis clinical scores were suppressed by each treatment. A flare in arthritis was then induced by intra-articular administration of 100U IL-1β. Mice were then treated either with a second subcutaneous injection of 5×10⁵ mBSA-exposed BAY DC or control immunoglobulin. The data show that suppression of arthritis was equivalent when comparing mBSA-exposed BAY DC, anti-TNFα or a combination of DC and anti-TNFα (see FIG. 16). Furthermore, arthritis could be resuppressed by mBSA-exposed BAY DC after a flare.

### Example 10

Administration of BAY DC to Arthritic Mice Alters the Isotype of Antigen-Specific Antibodies.

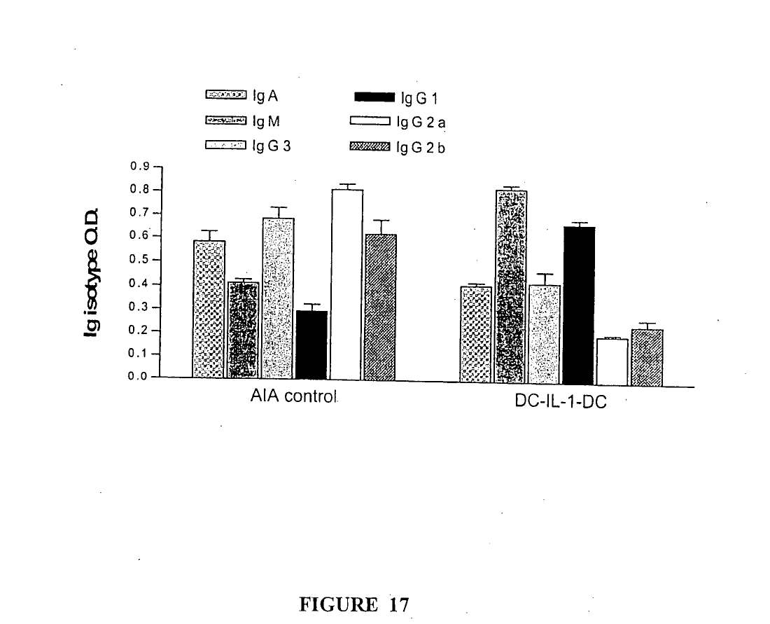

Sera obtained from mice in the preceding experiment were assessed for mBSA-specific antibodies by ELISA, and the isotype of the antibody was determined using isotype-specific detection reagents. Shown in FIG. 17 are the relative quantities of anti-mBSA AB of each isotype from control untreated mice or mice treated with mBSA-exposed BAY DC, followed by IL-1β flare, then retreated with mBSA-exposed BAY DC. The data indicate that BAY DC treatment reduces the relative proportion of the Th1 isotypes IgG2a, 2b and IgG3 and increases the relative proportion of the Th2 isotype IgG1, as well as increasing the relative proportion of IgM (suggesting a reduction in class switching to IgG) (FIG. 17).

### Example 11

Long Term Suppression of Antigen-Specific Responses by NFκB DC.

The duration of antigen-specific suppression of immunity was tested in mice primed with antigen KLH in complete Freund's adjuvant. Seven days later, groups of 5 mice were treated with either 5×10⁵ bone marrow derived DC generated in the presence (Bay DC) or absence of BAY 11-7082 and exposed or not to the antigen KLH, or with saline. A final group of mice was left unprimed and untreated.

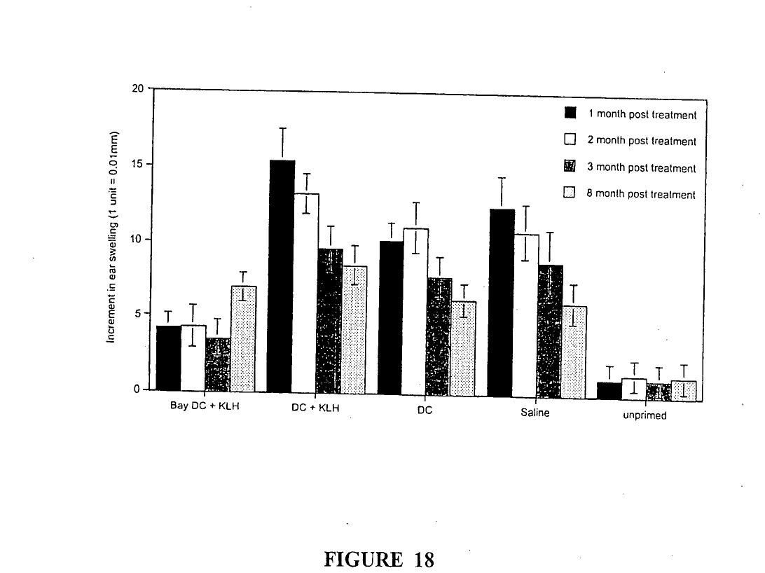

Between 1 and 8 months after DC or saline treatment, mice were tested for skin test reactivity (DTH) to KLH. Shown in FIG. 18 is the increment in ear swelling in response to KLH administered intradermally at each time point. There is no statistical difference in scores at any time point for mice administered Bay DC+KLH; p<0.01 comparing Bay DC with all other primed groups at 1 month time point. Therefore, suppression of antigen-specific responses remains for at least 8 months after a single administration of antigen-exposed Bay DC.

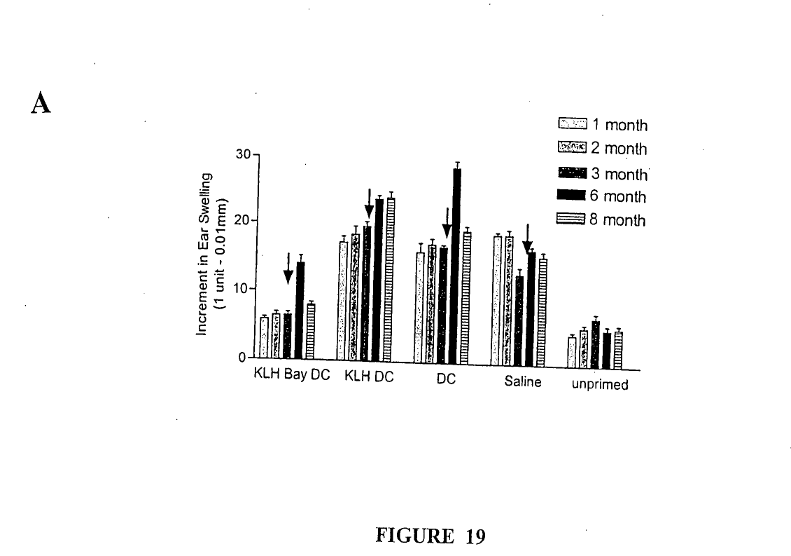

To further test the ability of Bay DC to suppress for long periods, the previous experiment was repeated twice, except that mice were boosted with KLH and complete Freund's adjuvant 1 week after the 3-month DTH analysis (arrows, FIG. 19A) or 1 week after the 1- and 3-month DTH analyses (arrows, FIG. 19B). Although skin test reactivity was enhanced somewhat by the boost with antigen and adjuvant, this increment was lower than that for mice previously treated with DC and KLH, and suppression was subsequently restored.

### Example 12

Suppression of Graft Versus Host Disease by BAY DC

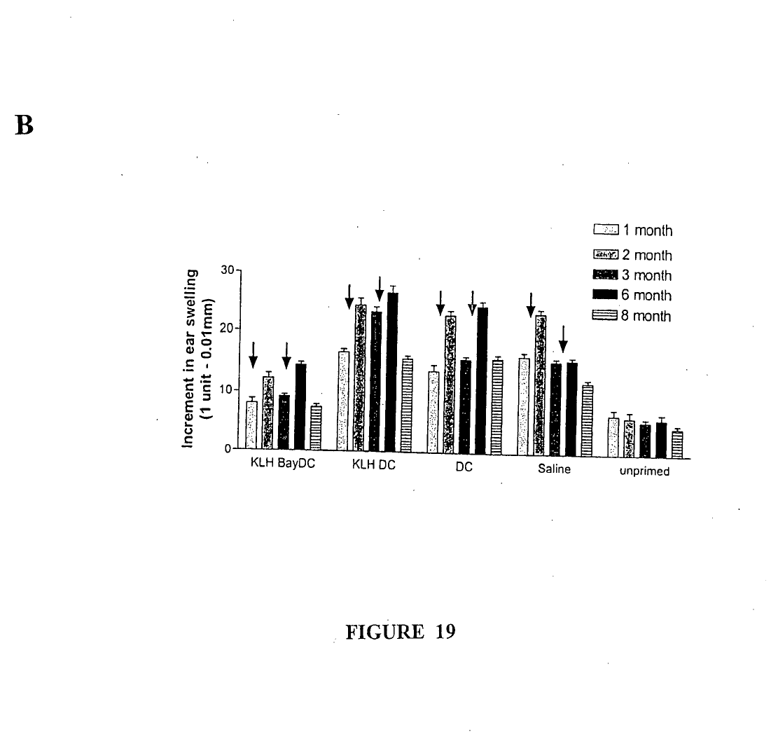

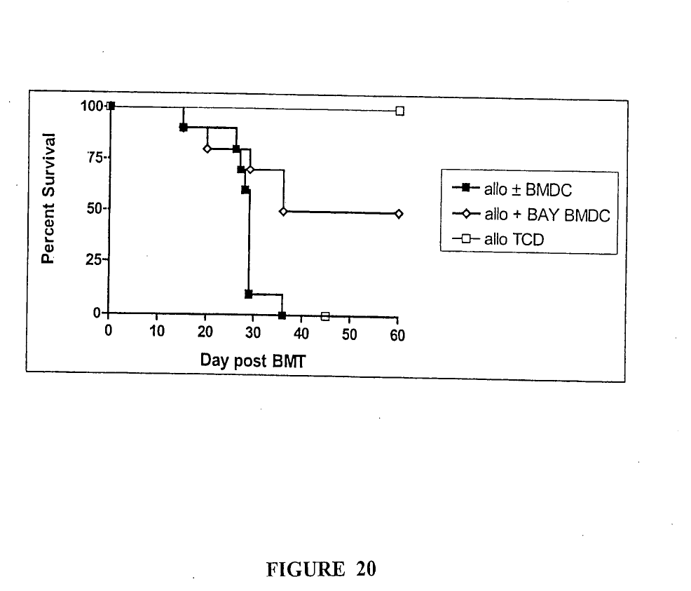

The effect of BAY DC on graft versus host disease (GVHD) was studied in a well described model in which lethally irradiated B6D2F1 recipient mice are transplanted with bone marrow and T cells from donor B6 mice (MacDonald et al., 2003, Blood 101:2033-2042.). This system is lethal in allogeneic BMT recipients by day 35 (FIG. 20; filled squares). The addition of 5×10⁵ of either donor or host control DC (BMDC) to allogeneic BM and T cells did not alter mortality (FIG. 20; filled squares). Animals that received 5×10⁵ donor (B6) or host (B6D2F1) BAY DC in addition to allogeneic BM and T cells had improved survival (FIG. 20; unfilled diamonds, p<0.02). All recipients of T cell depleted bone marrow (TCD) survived (FIG. 20; unfilled squares). Thus, survival of GVHD after transplantation of a fully MHC mismatched BM allograft can be improved by a single administration of a small number of donor or recipient BAY DC.

### Example 13

Prevention of Type I Diabetes in NOD Mice by NF-κB Dendritic Cells.

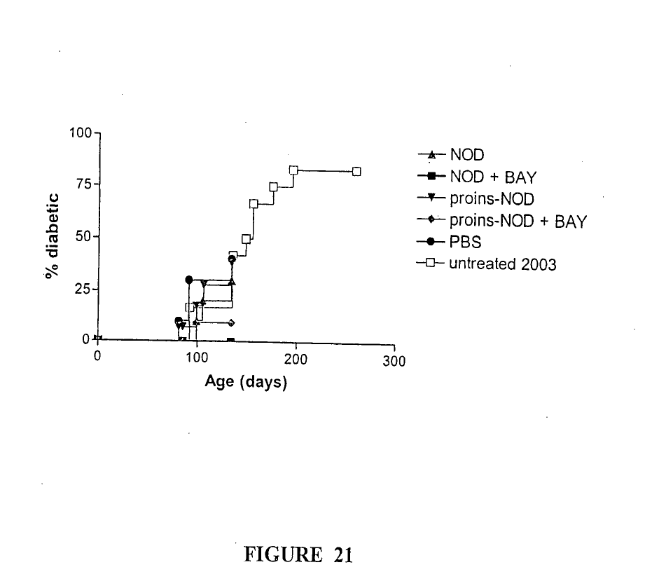

The non-obese diabetes mouse model of spontaneous type I diabetes mellitus was used to assess the capacity of Bay DC to prevent autoimmune disease. Four-week old female NOD mice were treated once with 5×10⁵ dendritic cells, administered subcutaneously. Dendritic cells were generated either from syngeneic NOD bone marrow, or from bone marrow cells from NOD mice transgenic for the autoantigen proinsulin, driven by the invariant chain promoter, each in the presence or absence of BAY11-7082. Onset of diabetes commences around 85 days, with 80-90% diabetic within 200 days. Treatment of mice with Bay DC generated from either NOD or proinsulin-NOD bone marrow cells reduces the rate of onset of diabetes at 135 days, when compared with untreated, saline-treated or dendritic cell treated mice (FIG. 21).

### Example 14

The Phenotype of NF-κB Dendritic Cells is Reproducible with Different NF-κB Inhibitors.

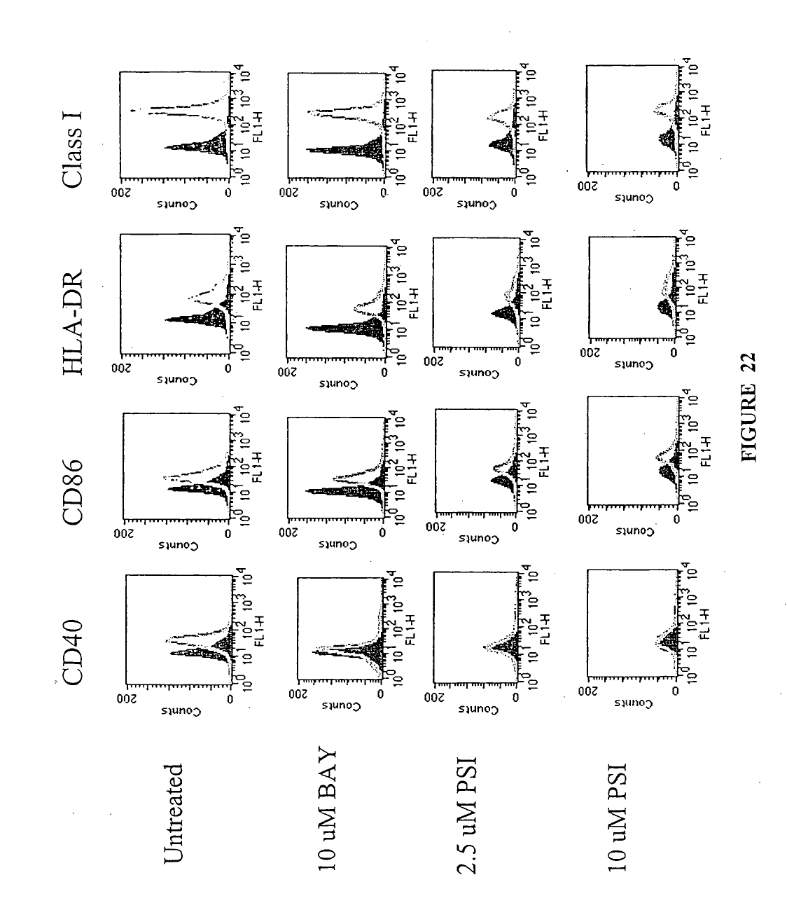

The present inventors examined whether the phenotype of the described dendritic cells that are capable of inducing tolerance when transferred in vivo, is reproducible when dendritic cells are generated with various NT-κB⁻ inhibitors. Dendritic cells were generated from human monocles for 48 hours in the presence of GM-CSF, IL-4 and in the presence or absence of either 10 κM of the IκB phosphorylation inhibitor BAY11-7082 or 2.5 or 10 μM of the proteasome inhibitor PSI (N-benzyloxycarbonyl-Ile-Glu(O-tert-butyl)-Ala-leucinal). As shown in FIG. 22, the phenotype of dendritic cells generated in the presence of either inhibitor was indistinguishable, in that CD40 expression was suppressed, HLA-DR was reduced and CD86 and MHC class I expression were not changed, relative to immature DC generated in the absence of inhibitors.

### Example 15

Antigen-Specific Tolerance Induced by NF-κB Macrophages

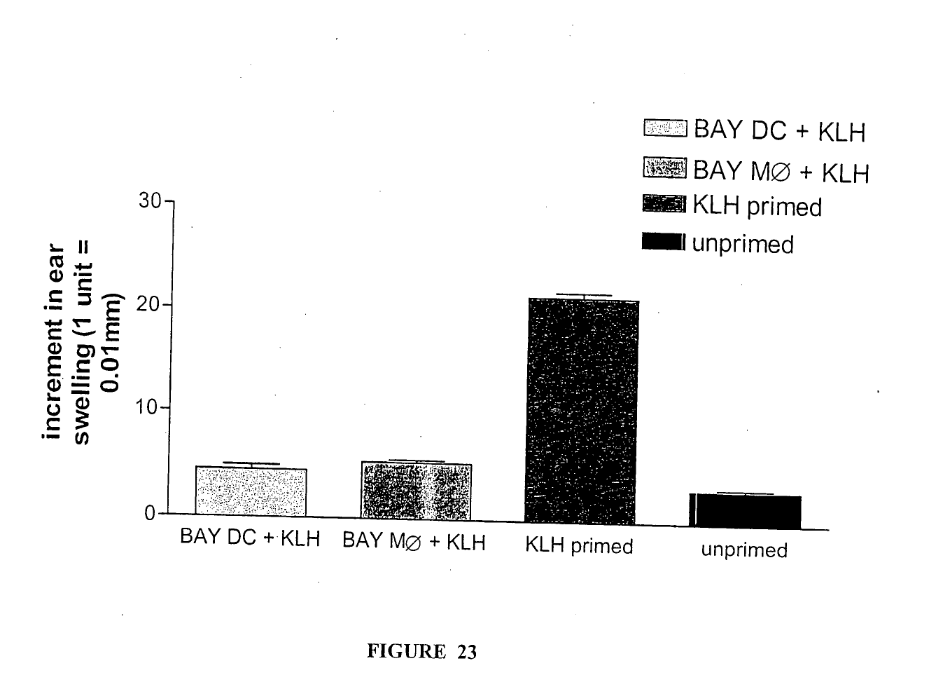

To determine whether dendritic cells were the only antigen presenting cells capable of inducing antigen-specific tolerance when NF-κB activation was blocked using BAY11-7082, antigen presenting function of bone marrow derived dendritic cells and macrophages was compared in vivo. For the generation of macrophages, bone marrow cells were depleted of red cells by ficoll gradient centrifugation then cultured with 1×10⁴ U/ml CSF-1 for 7 days as described by Stacey et al., 1996, J. Immunol. 157: 2116-2122. Dendritic cells were generated from bone marrow as previously described by Martin et al., 2003, Immunity 18: 155-167. Dendritic cells or macrophages were generated from bone marrow precursors in the presence of growth factors and 10 μM Bay11-7082 then washed and exposed to the antigen KLH. Recipient mice were primed with KLH in CFA, then administered 5×10⁵ DC or macrophages subcutaneously or no treatment. One group was not primed with KLH in CFA. KLH-specific delayed type hypersensitivity responses were measured on all groups 4 days after administration of antigen presenting cells. The results presented in FIG. 23 clearly show that bone marrow macrophages also induce antigen-specific tolerance.

Conclusions

The Examples described above show that antigen-exposed myeloid DCs and macrophages, in which NF-κB function is inhibited, lack cell surface CD40 expression, prevent priming of immunity, and suppress a previously primed immune response. DCs or macrophages in which RelB nuclear translocation is inhibited through prevention of IκB phosphorylation, or in which proteolysis is inhibited, as well as DCs generated from RelB deficient mice, and DCs generated from CD40 deficient mice similarly conferred suppression.

Thus, in contrast to published hypotheses concerning DC involvement in tolerogenic responses, CD40 regulated by NF-κB activity—as opposed to “DC immaturity”—determines the consequences of presentation of antigen by myeloid DCs. In this regard, DCs in which NF-κB activity was suppressed during development from BM precursors, or CD40 deficient DCs expressed levels of CD86 equivalent to those of mature DCs, and higher than those expressed by immature DCs. Furthermore, CD40 levels were lower than those expressed by immature BM DCs, and the expression and function of CD40 and NF-κB could not be induced by inflammatory signals such as LPS or CD40 ligand. Thus, while in vitro-derived immature BM DCs share some characteristics of the BAY-treated BMDCs, including modest induction of tolerance, the current data indicate that deficiency in NF-κB activity, especially RelB activity, leads to the generation of DCs with a unique phenotype. Recently, the phenotype and viability of BMDCs generated from RelA, c-Rel and p50 deficient mice were demonstrated (Ouaaz et al., 2002). Of interest, BMDCs generated from RelA/p50 doubly deficient mice were more prone to death, and BMDCs generated from c-Rel/p50 deficient mice showed intact CD40 expression and APC function in MLR (when corrected for variability), but reduced IL-12 production. Additionally, LPS-induced up-regulation of MHC molecules, ICAM-1, CD80 and CD86 was unaffected in c-Rel/p50 deficient mice. Taken together with the current and with previous studies, these data indicate that RelA, RelB and c-Rel—partnering with p50—each play unique and complementary roles in the process of myeloid DC differentiation (Grumont et al., 2001; Neumann et al., 2000; O'Sullivan and Thomas, 2002; Rescigno et al., 1998). In particular, RelB/p50 specifically controls functional myeloid DC differentiation and CD40 expression. A similar role for RelB and CD40 in determining the consequences of presentation of antigen by B cells has also emerged (Buhlmann et al., 1995; Hollander et al., 1996. O'Sullivan et al., 2000; Pai et al., 2002).

Induction of suppression was specific for the antigen to which DCs had been exposed. Moreover, this suppression results at least in part from induction of antigen-specific regulatory T cells (Treg), as DC Immunisation increased the proportion of CD4⁺ T cells producing IL-10 in draining LN, and CD4⁺ splenic T cells from tolerant animals transferred antigen-specific tolerance to primed recipients in an IL-10-dependent manner. Therefore, the DCs induced an active “infectious” process of antigen-specific regulation (Cobbold and Waldmann, 1998). While the exact phenotype of the CD4⁺ Treg induced in the above Examples is not yet elucidated, they most closely resemble Tr1 cells. Antigen specific Tr1 cells induced by monocytes in vitro produce IL-10, and are able to suppress inflammation in colitis and allergic models in an IL-10 dependent manner (Cottrez et al., 2000; Groux et al., 1997). In keeping with the current studies demonstrating that suppression of primed immune responses by DCs correlates with their RelB nuclear binding activity and CD40 expression, human immature monocyte-derived myeloid DCs also induced CD8⁺ T regulatory cells in vivo, which produced high levels of IL-10 and low levels of IFN-γ but no IL-4 (Dhodapkar et al., 2001).

These observations have significance for immunotherapeutic suppression of conditions in which ongoing antigen presentation is associated with chronic inflammation, including autoimmune disease, allograft rejection and graft-versus-host-disease. Of importance, in the current studies, induced Treg were capable of traffic from draining LN to spleen following s.c. administration of DCs. After adoptive transfer, the Treg are likely to suppress DTH responses locally in the skin, but also suppress T cell proliferation in LN draining the site of antigen priming in the recipient animals.

The NF-κB family of proteins is regulated by IκB and other inhibitory molecules in the cytosol (Baldwin, 1996). Cellular activation leads to IκB phosphorylation and translocation of active NF-κB to the nucleus. RelB is translocated upon myeloid DC differentiation and it heterodimerizes with p50 in the DC nucleus (Neumann et al., 2000; O'Sullivan and Thomas, 2002; Pettit et al., 1997). RelB and p50 deficient mice exhibit multiple deficits in immune function—in particular, RelB deficient mice lack mature myeloid DCs and the liver and spleen are infiltrated by myeloid cells, including monocytes, granulocytes and progenitor-cells (Burkly et al., 1995; Sha et al., 1995; Weih et al., 1995). The role of NF-κB in DC APC function was previously examined in vivo through the use of NF-κB decoy oligonucleotides (Giannoukakis et al., 2000). However, in these studies NF-κB inhibition was commenced several days after the initiation of the DC cultures and CD40 expression by BMDCs was not affected by the NF-κB decoy. BAY 11-7082 has been shown previously to block NF-κB nuclear translocation through inhibition of IκB phosphorylation (O'Sullivan and Thomas, 2002: Pierce et al., 1997). While nuclear translocation of all NF-κB subunits was inhibited in BMDCs, the specificity of the drug for NF-κB is unknown. However, the present inventors provide two additional pieces of evidence that the consequences of antigen presentation by myeloid DCs are indeed determined by RelB activity. Thus, RelB deficient BMDCs conferred similar suppression to BMDCs generated from wild type mice in the presence of the BAY 11-7082 inhibitor. Furthermore, the extent of nuclear RelB DNA binding in DCs was inversely correlated with the induction of suppression by those cells.

The current data highlight the potential for development of antigen-specific autoimmune immunotherapy using DCs or macrophages treated with inhibitors of NF-κB, in view of the potent suppression of CD40 expression without the need for genetic manipulation of these antigen-presenting cells, and the profound effect on previously primed immune responses in vivo.

Experimental Procedures

Culture Medium

DCs were cultured in RPMI, supplemented with 10% heat-inactivated fetal calf serum (FCS, CSL, Parkville, Australia), 100 μg/ml penicillin, 100 μg/ml streptomycin, 10 mM sodium pyruvate, 20 mM HEPES, 2 mM L-glutamine and 50 μM 2-mercaptoethanol (culture medium, CM). In all experiments where methylated BSA (mBSA) is used as the challenge antigen, DCs were cultured in serum free Excell 620 culture medium (CSL Biosciences) supplemented with 100 μg/mL penicillin, 100 μg/mL streptomycin, 10 mM sodium pyruvate, 20 mM HEPES, 2 mM L-glutamine and 50 μM 2-mercaptoethanol (Excell).

Bone Marrow Derived Dendritic Cells (BMDCs).

Bone marrow cells were collected from murine long bones, suspended by vigorous pipetting, passed through nylon mesh, and the mononuclear cells separated by ficoll gradient centrifugation. Macrophages, class II cells and lymphocytes were immunodepleted using appropriate mAb followed by magnetic beads (MACS, Miltenyi Biotec, Calif.). BM cells were incubated for 6-8 days in CM supplemented with 0.5 ng/mL recombinant murine GM-CSF and 0.5 ng/mL recombinant murine IL-4 (both from Peprotech, USA) and fresh medium was applied every second day. Resulting preparations routinely contained 80-90% CD11c⁺ cells. Some BMDCs were cultured continuously in the presence of 5 μM BAY 11-7082 (BioMol, Plymouth Meeting, Pa.) and washed three times before use. For immature BMDCs, identically treated BM cells were incubated for 8 d in CM, supplemented with 0.5 ng/ml recombinant murine GM-CSF (Peprotech).

Flow Cytometry

BMDCs were incubated for 30 min on ice with anti-CD86-FITC (GL1, PharMingen, San Diego, Calif.), anti-CD11c-PE (HL3, PharMingen), anti-CD11b-PE (M1/70, PharMingen), F4/80-PE (Serotec, Raleigh, N.C.), CD8α-PE (53-6.7, PharMingen), CD3-PE (17A2, PharMingen), or with anti-CD40 (3/23, PharMingen), anti-CD19 (1D3, American Type Culture Collection, ATCC), anti-Ly6-g and c (Gr1, RB6-BC5), anti-CD45R (B220, RA3-6B2, each a gift from G. Hill, Queensland Institute for Medical Research, Brisbane, Q1d), anti-MHC class I and anti-MHC class II (2G9, PharMingen), followed by biotinylated rabbit anti-rat Ig (DAKO, CA) and then streptavidin-FITC (DAKO). After washing, cells were analyzed using a FACScalibur (Becton Dickinson, San Jose, Calif.). Intracellular cytokine production was measured in lymphocytes by flow cytometric staining as described, with minor modifications (Sander et al., 1991). Briefly, lymphocytes were stimulated in the presence of PMA/Ionomycin and brefeldin A for IS h, then stained with FITC-CD4 (Pharmingen), followed by 4% paraformaldehyde fixation and permeabilization with saponin (Sigma, Mo.). Permeabilized cells were stained with allophycocyanin (APC) labeled-anti-IL-10, APC-anti-IL-4, or APC-anti-IFN-γ (Pharmingen) for 30 min on ice and washed twice.

Protein Extraction and Immunoblotting

Nuclear and cytoplasmic extracts were prepared as previously described (Pettit et al., 1997) and protein estimations carried out using a Protein Assay kit (Bio-Rad, Hercules, Calif.). 10 μg of protein extract were separated by 8% SDS-PAGE. Following transfer to nitrocellulose (Amersham. Sussex, England), membranes were immunoblotted with either anti-RelB (sc-226), anti-p50 (sc-7178), anti-c-Rel, anti-RelA, or anti-p52 antibodies (all from Santa Cruz Biotechnology) followed by sheep anti-rabbit HRP-conjugated Ig (Silenus, Hawthorn, Australia) and then detected by enhanced chemiluminescence (ECL, Life Technologies, MO, USA) according to the manufacturer's instructions (Amersham).

NF-κB Binding ELISA

p50 and RelB DNA binding was detected by ELISA using a Mercury Transfactor p50 Kit (Clontech, Calif.). 10 μg of nuclear extract were bound to wells coated with NF-κB consensus oligonucleotide then incubated with either anti-RelB (sc-226) or anti-p50 (sc-7178), followed by anti-rabbit HIP-conjugated Ig (Silenus) and then detected by measuring color development of TMB at 650 nm using a Multiskan plate reader (Labsystems) (O'Sullivan and Thomas, 2002).

Mice and Immunisation

C57BL/6 and BALB/c mice (Animal Resource Centre, Perth, Australia) were maintained in specific pathogen free (SPF) conditions. The RelB mutant C57BL/6 mice were originally generated in D.Lo's laboratory (Burkly et al., 1995). They were bred under SPF conditions in the animal facility of the Walter and Eliza Hall Institute (WEHI). Homozygous RelB^{-/-} mice were selected and supplied by Dr L. Wu (WEHI) at 5-7 weeks of age for BMDC generation. CD40 deficient mice (Kawabe et al., 1994) were crossed for over 10 generations under SPF conditions with BALB/c mice at the animal facility at Australian National University (ANU), and homozygous CD40^{-/-} mice were supplied from ANU at 5 weeks of age. IL-10 and MHC class U deficient C57B1/6 mice were bred under SPF conditions and supplied from ANU at 6 weeks of age. All s.c. injections were delivered to the tailbase. Mice were immunised s.c. with 60 μg of mBSA or 50 kg of keyhole limpet hemocyanin (KLH) or 50 μg of ovalbumin in complete Freunds adjuvant (CFA). 5×10⁵ BMDCs were injected s.c. or i.v., 7 days before or after the immunisation. Serum antigen-specific Ab, draining lymph node (DLN) T-cell proliferative responses and DTH responses were measured. For the DTH responses, mice were injected i.d with either 5 μg of antigen or saline into the ears and ear swelling was measured and scored 24 h later using an engineer's micrometer. For the adoptive transfer experiments, C57BL/6 mice were injected s.c. with 5×10⁵ KLH-pulsed BMDCs. Spleens were removed 7 days later and splenocytes were enriched for T cells by transfer to sterile nylon wool columns (Robbins Scientific, Sunnyvale, Calif.) for 1 h at 37° C. In some experiments, CD3⁺ CD4⁺ and CD3⁺ CD4⁻ cells were sorted using a Moflo flow cytometer after staining with anti-CD3-FITC and anti-CD4-PE (Cytomation, Fort Collins, Colo.). Purity was approximately 85%. In other experiments, CD3⁺ CD4⁺ T cells were purified by immunomagnetic depletion with anti-CD8, anti-MHC class II, Gr1, B220, and F4/80. 2.5-5×10⁵ of each purified population was injected i.v. into non-irradiated syngeneic recipients, primed 7-9 days previously with either KLH or ovalbumin in CFA. Antigen-specific T cell proliferative responses were measured in DLN after 7 days.

In Vitro Proliferative and Antibody Responses

For the T-cell proliferation assay, a single cell suspension was prepared from the inguinal LNs. 4×10⁵ LN, cells/well were incubated in triplicate in the presence or absence of varying concentrations of antigen or 1 μg Concanavalin A (Con A, Sigma, Mo.), at 37° C. in 5% CO₂ for 3 days. In all experiments where methylated BSA (mBSA) is used as the challenge antigen, assays were carried out in serum free Excell 620 culture medium. Cells were pulsed with 1 μCi ³H-thymidine/well for the final 6-8 h, then harvested onto glass fibre filters using an automated cell harvester. Incorporated ³H-thymidine was counted using a Packard TopCount NXT (Packard, Meriden, Conn.). Specific ³H-thymidine incorporation (cpm) was the mean±SEM of triplicate wells.

For mBSA or KLH-specific Ab determination by ELISA, mice were bled from the lateral tail vein and serum prepared. 100 μL of mBSA or KLH protein, at 10 μg/mL in 50 mM carbonate buffer (pH 9.6), was coated onto the wells of 96 well microtitre plates (Griener Labortechnik, Kresmuster, Austria). After washing with 0.5% Tween 20/PBS and blocking with 200 μL 3% BSA fraction V, 100 μl serum in five-fold dilutions were added to triplicate wells. After washing, each well was incubated with 100 μL of biotinylated rabbit anti-mouse Ig secondary antibody (DAKO), followed by washing and incubation with streptavidin-horseradish peroxidase. After incubation with 0.1% ABTS in 0.03% hydrogen peroxide and 150 mM citrate buffer, pH 4.5, the presence of antigen-specific antibodies was detected by the net absorbance readings at 405 nm and 492 nm.

Statistical Analysis

Differences were compared using Students t tests. Differences were considered significant at p<0.05.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1
Cytokine production by T cells in draining LN

| Ex-vivo treatment | % CD4*IL-10⁺ cells | % CD4⁺IFNγ⁺ cells |
|---|---|---|
| EXPERIMENT 1, H-2^{B} MICE | PMA in vitro (mean % ± SD) | |
| DCs | 0.62 ± 0.22 | 0.8 ± 0.11 |
| DCs + KLH | 0.52 ± 0.15 | 2.5 ± 0.62 |
| DCs + BAY + KLH | 11.23 ± 0.18 | 1.62 ± 0.29 |
| RelB^{-/-} DCs + KLH | 3.5 ± 0.05 | 0.7 ± 0.11 |
| EXPERIMENT 2, H-2^{D} | | |
| MICE | | |
| DCs | 0.9 ± 0.8 | 1.3 ± 0.2 |
| DCs + KLH | 1.1 ± 0.5 | 3.9 ± 0.8 |
| CD40^{-/-} DCs + KLH | 4.7 ± 0.2 | 1.8 ± 0.5 |
| CD40^{-/-} DCs + BAY + KLH | 5.4 ± 0.01 | 2.7 ± 0.2 |
| EXPERIMENT 3, H-2^{B} MICE | KLH in vitro (mean % ± SD) | |
| DCs | 0.1 ± 0.12 | 2.17 ± 0.01 |
| DCs + KLH | 0.66 ± 0.03 | 2.5 ± 0.15 |
| DCs + BAY + KLH | 2.98 ± 0.13 | 0.1 ± 0.02 |

H-2^{b} mice were injected with KLH-pulsed DCs generated from RelB^{+/+} or RelB^{-/-} BM in the presence or absence of BAY (experiment 1), or H-2^{d} mice were injected with KLH-pulsed DCs generated from CD40^{+/+} or CD40^{-/-} BM in the presence or absence of BAY (experiment 2). H-2^{b} mice were injected with KLH-pulsed DCs generated from BM in the presence or absence of BAY (experiment 3). Control mice were injected with DCs alone. After 5 days, DLN cells were stimulated with either PMA/Ionomycin (experiments 1, 2), KLH, or medium alone each in the absence of serum (experiment 3) for 18 h in the presence of brefeldin A then stained with CD4-PE and either IL-10-APC or IFN-γ-APC. Mean±SD % cytokine-expressing CD4⁺ T cells from groups of 5 mice tested individually are shown.

### BIBLIOGRAPHY

Anton, D., Dabadghao, S., Palucka, K., Holm, G. & Yi, Q. (1998). Generation of dendritic cells from peripheral blood adherent cells in medium with human serum. Scand J Immunol 47, 116-121.
Albeit M L, Sauter B, Bhardwaj N. (1998). Dendritic cells acquire antigen from apoptotic cells and induce class I-restricted CTLs. Nature 392(6671), 86-89
Araki, H., Katayama, N., Mitani, H., Suzuki, H., Nishikawa, H., Masuya, M., Ikuta, Y., Hoshino, N., Miyashita, H., Nishii, K., Minami, N. & Shiku, H. (2001). Efficient ex vivo generation of dendritic cells from CD14+ blood monocytes in the presence of human serum albumin for use in clinical vaccine trials. Br J Haematol 114, 681-689.
Ardavin, C., Wu, L., Li, C. L., and Shortman, K. (1993). Thymic dendritic cells and T cells develop simultaneously in the thymus from a common precursor population, Nature 362, 761-763.
Bai, L., Feuerer, M., Beckhove, P., Umansky, V. & Schirrmacher, V. (2002). Generation of dendritic cells from human bone marrow mononuclear cells: advantages for clinical application in comparison to peripheral blood monocyte derived cells. Int J Oncol 20, 247-53.
Baldwin, A. S., Jr. (1996). The NF-kappa B and I kappa B proteins: new discoveries and insights, Annu Rev Immunol 14, 649-683.
Banchereau, J., Briere, F., Caux, C., Davoust, J., Lebecque, S., Liu, Y. J., Pulendran, B., and Palucka, K. (2000). Immunobiology of dendritic cells, Annu Rev Immunol 18, 767-811.
Basham, T., Smith, W., Lanier, L., Morhenn, V. & Merigan, T. (1984). Regulation of expression of class II major histocompatibility antigens on human peripheral blood monocytes and langerhans cells by interferon. Human Immunology 10, 83-93.
Battye, F. L. & Shortman, K. (1991). Flow cytometry and cell-separation procedures. [Review]. Curr. Opin. Immunol. 3, 238-241.
Bouchon, A., Facchetti, F., Weigand, M. A., and Colonna, M. (2001a). TREM-1 amplifies inflammation and is a crucial mediator of septic shock, Nature 410, 1103-1107.
Bouchon, A., Hernandez-Munain, C., Cella, M., and Colonna, M. (2001b). A DAP12-mediated pathway regulates expression of CC chemokine receptor 7 and maturation of human dendritic cells, J Exp Med 194, 1111-1122.
Boyum, A. (1968). Isolation of mononuclear cells and granulocytes from human blood: isolation of mononuclear cells by one centrifugation and of sedimentation at 1 g. Scan J of Clin and Lab Invest 21(S97), 77.
Buhlmann, J. E., Foy, T. M., Aruffo, A., Crassi, K. M., Ledbetter, J. A., Green, W. R., Xu, J. C., Shultz, L. D., Roopesian, D., Flavell, R. A., and et al. (1995). In the absence of a CD40 signal, B cells are tolerogenic, Immunity 2, 645-653.
Burkly, L., Hession, C., Ogata, L., Reilly, C., Marconi, L. A., Olson, D., Tizard, R., Cate, R., and Lo, D. (1995). Expression of relB is required for the development of thymic medulla and dendritic cells, Nature 373, 531-536.
Caamano, J., Alexander, J., Craig, L., Bravo, R., and Hunter, C. A. (1999). The NF-kappa B family member RelB is required for innate and adaptive immunity to Toxoplasma gondii, J Immunol 163, 4453-61.
Caux, C., Massacrier, C., Vanbervliet, B., Dubois, B., van Kooten, C., Durand, I., and Banchereau, J. (1994). Activation of human dendritic cells through CD40 cross-linking, J Exp Med 180, 1263-1272.
Cavanagh L L, Saal R J, Grimmett K L, Thomas R. (1998). Proliferation in monocyte-derived dendritic cell cultures is caused by progenitor cells capable of myeloid differentiation. Blood 92(5), 1598-1607.
Cella, M., Scheidegger, D., Palmer Lehmann, K., Lane, P., Lanzavecchia, A., and Alber, G. (1996). Ligation of CD40 on dendritic cells triggers production of high levels of interleukin-12 and enhances T cell stimulator capacity: T-T help via APC activation, J Exp Med 184, 747-752.
Chen, B., Stiff, P., Sloan, G., Kash, J., Manjunath, R., Pathasarathy, M., Oldenburg, D., Foreman, K. E. & Nickoloff, B. J. (2001). Replicative response, immunophenotype, and functional activity of monocyte-derived versus CD34(+)-derived dendritic cells following exposure to various expansion and maturational stimuli. Clin Immunol 98, 280-292.
Clark, R. B. (2002). The role of PPARs in inflammation and immunity. J Leukoc Biol, 71, 388-400.
Cobbold, S., and Waldmann, H. (1998). Infectious tolerance, Curr Opin Immunol 10, 518-524.
Corinti S, Medaglini D, Cavani A, Rescigno M, Pozzi G, Ricciardi-Castagnoli P, Girolomoni G. (1999). Human dendritic cells very efficiently present a heterologous antigen expressed on the surface of recombinant gram-positive bacteria to CD4⁺ T lymphocytes. J Immunol 163(6), 3029-3036.
Cottrez, F., Hurst, S. D., Coffman, R. L., and Groux, H. (2000). T regulatory cells 1 inhibit a Th2-specific response in vivo, J Immunol 165, 4848-4853.
Davis, L. S., McIlraith, M. J., Paecheo, T., Becker, B., Adix, L. M., Thomas, R., Wacholtz, M. C. & Lipsky, P. E. (1994). Assessment of a positive selection technique using an avidin column to isolate human peripheral blood T cell subsets. J Immunol Meth 175, 247-257.
de Jong, E. C., Vieira, P. L., Kalinski, P., and Kapsenberg M. L. (1999). Corticosteroids inhibit the production of inflammatory mediators in immature monocyte-derived DC and induce the development of tolerogenic DC3, J Leukoc Biol 66, 201-204.
del Hoyo, G. M., Martin, P., Vargas, H. H., Ruiz, S., Arias, C. F. & Ardavin, C. (2002). Characterization of a common precursor population for dendritic cells. Nature 415, 1043-1047.
Dhodapkar, M. V., Steinman, R. M., Krasovsky, J., Munz, C., and Bhardwaj, N. (2001). Antigen-specific inhibition of effector T cell function in humans after injection of immature dendritic cells, J Exp Med 193, 233-238.
Diehl, L., Den Boer, A. T., van der Voort, E. I., Melief, C. J., Offringa, R., and Toes, R. E. (2000). The role of CD40 in peripheral T cell tolerance and immunity, J Mol Med 78, 363-371.
Erickson-Miller, C. L., Brennan, J. K. & Abboud, C. N. (1990). Examination of survival, proliferation and cell surface antigen expression of human monocytes exposed to macrophage colony-stimulating factor (M-CSF). Int J Cell Cloning 8, 346-356.
Farkas, L., Beiske, K., Lund-Johansen, F., Brandtzaeg, P. & Jahnsen, F. L. (2001). Plasmacytoid Dendritic Cells (Natural Interferon-alpha/beta-Producing Cells) Accumulate in Cutaneous Lupus Erythematosus Lesions. Am J Pathol 159, 237-243.
Fearnley, D. B., Whyte, L. F., Carnoutsos, S. A., Cook, A. H. & Hart, D. N. (1999). Monitoring human blood dendritic cell numbers in normal individuals and in stem cell transplantation. Blood 93, 728-736.
Feldmann, M. (2001). Pathogenesis of arthritis: recent research progress, Nat Immunol 2, 771-773.
Feldmann, M., and Maini, R. N. (2001). Anti-TNFa therapy of rheumatoid arthritis: What have we learned?, Annu Rev Immunol 19, 163-196.
Feuerstein, B., Berger, T. G., Maczek, C., Roder, C., Schreiner, D., Hirsch, U., Haendle, I., Leisgang, W., Glaser, A., Kuss, O., Diepgen, T. L., Schuler, G. & Schuler-Thurner, B. (2000). A method for the production of cryopreserved aliquots of antigen-preloaded, mature dendritic cells ready for clinical use. J Immunol Methods 245, 15-29.
Fleming, W. H., Mulcahy, J. M., McKearn, J. P. & Streeter, P. R. (2001). Progenipoietin-1: a multifunctional agonist of the granulocyte colony-stimulating factor receptor and fetal liver tyrosine kinase-3 is a potent mobilizer of hematopoietic stem cells. Exp Hematol 29, 943-951.
Freudenthal, P. S. & Steinman, R. M. (1990). The distinct surface of human blood dendritic cells, as observed after an improved isolation method. Proc Natl Acad Sci USA 87, 7698-7702.
Gao, J. X., Madrenas, J., Zeng, W., Cameron, M. J., Zhang, Z., Wang, J. J., Zhong, R., and Grant, D. (1999). CD40-deficient dendritic cells producing interleukin-10, but not interleukin-12, induce T-cell hyporesponsiveness in vitro and prevent acute allograft rejection, Immunology 98, 159-170.
Garderet, L., Cao, H., Salamero, J., Verge, V., Tisserand, E., Scholl, S., Gorin, N. C. & Lopez, M. (2001). In vitro production of dendritic cells from human blood monocytes for therapeutic use. J Hematother Stem Cell Res 10, 553-567.
Geissmann, F., Prost, C., Monnet, J. P., Dy, M., Brousse, N. & Hermine, O. (1998). Transforming growth factor beta 1, in the presence of granulocyte/macrophage colony-stimulating factor and interleukin 4, induces differentiation of human peripheral blood monocytes into dendritic Langerhans cells. J Exp Med, 187, 961-966.
Giannoukakis, N., Bonham, C. A., Qian, S., Zhou, Z., Peng, L., Harnaha, J., Li, W., Thomson, A. W., Fung, J. J., Robbins, P. D., and Lu, L. (2000). Prolongation of cardiac allograft survival using dendritic cells treated with NF-κB decoy oligodeoxyribonucleotides, Mol Ther 1, 430-437.
Goodnow, C. C. (2001). Pathways for self-tolerance and the treatment of autoimmune diseases, Lancet 357, 2115-2121.
Gregori, S., Casorati, M., Amuchastegui, S., Smiroldo, S., Davalli, A. M., and Adorini, L. (2001). Regulatory T cells induced by 1alpha,25-dihydroxyvitamin d(3) and mycophenolate mofetil treatment mediate transplantation tolerance, J Immunol 167, 1945-1953.
Griffin, M. D., Lutz, W., Phan, V. A., Bachman, L. A., McKean, D. J., and Kumar, R. (2001). Dendritic cell modulation by 1alpha,25 dihydroxyvitamin D3 and its analogs: a vitamin D receptor-dependent pathway that promotes a persistent state of immaturity in vitro and in vivo, Proc Natl Acad Sci USA 98, 6800-6805.
Groopman, J. E., Mitsuyasu, R. T., DeLeo, M. J., Oette, D. H. & Golde, D. W. (1987). Effect of recombinant human granulocyte-macrophage colony-stimulating factor on myelopoiesis in the acquired immunodeficiency syndrome. N Engl J Med 317, 593-598.
Grouard, G., Rissoan, M. C., Filgueira, L., Durand, I., Banchereau, J. & Liu, Y. J. (1997). The enigmatic plasmacytoid T cells develop into dendritic cells with interleukin (IL)-3 and CD40-ligand. J Exp Med 185, 1101-1111.
Groux H, Bigler M, de Vries J E, Roncarolo M G. (1996). Interleukin-10 induces a long-term antigen-specific anergic state in human CD4⁺ T cells. J Exp Med 184(1):19-29.
Groux, H., O'Garra, A., Bigler, M., Rouleau, M., Antonenko, S., de Vries, J. E., and Roncarolo, M. G. (1997). A CD4⁺ T-cell subset inhibits antigen-specific T-cell responses and prevents colitis, Nature 389, 737-742.
Grumont, R., Hochrein, H., O'Keeffe, M., Gugasyan, R., White, C., Caminschi, I., Cook, W., and Gerondakis, S. (2001). c-Rel regulates interleukin 12 p70 expression in CD8(+) dendritic cells by specifically inducing p35 gene transcription, J Exp Med 194, 1021-1032.
Hacker, G., Redecke, V. & Hacker, H. (2002). Activation of the immune system by bacterial CpG-DNA. Immunology 105, 245-251.
Hackstein, H., Morelli, A. E., Larregina, A. T., Ganster, R. W., Papworth, G. D., Logar, A. J., Watkins, S. C., Falo, L. D., and Thomson, A. W. (2001). Aspirin inhibits in vitro maturation and in vivo immunostimulatory function of murine myeloid dendritic cells, J Immunol 166, 7053-7062.
Hanninen, A., Martinez, N. R., Davey, G. M., Heath, W. R., and Harrison, L. C. (2002). Transient blockade of CD40 ligand dissociates pathogenic from protective mucosal immunity, J Clin Invest 109, 261-267.
Hart D N, Fabre J W. (1981). Demonstration and characterization of Ia-positive dendritic cells in the interstitial connective tissues of rat heart and other tissues, but not brain. J Exp Med 154(2), 347-361.
Hart D N, McKenzie J L. (1988). Isolation and characterization of human tonsil dendritic cells. J Exp Med 168(1), 157-170.
Hofer, S., Rescigno, M., Granucci, F., Citterio, S., Francolini, M., and Ricciardi-Castagnoli, P. (2001). Differential activation of NF-kappa B subunits in dendritic cells in response to Gram-negative bacteria and to lipopolysaccharide, Microbes Infect 3, 259-265.
Hill, A. D., Naama, H. A., Calvano, S. E. & Daly, J. M. (1995). The effect of granulocyte-macrophage colony-stimulating factor on myeloid cells and its clinical applications. J Leukoc Biol 58, 634-642.
Hollander, G. A., Castigli, E., Kulbacki, R., Su, M., Burakoff, S. J., Gutierrez-Ramos, J. C., and Geha, R. S. (1996). Induction of alloantigen-specific tolerance by B cells from CD40-deficient nice, Proc Natl Acad Sci USA 93, 4994-4998.
Huang, F. P., Platt, N., Wykes, M., Major, J. R., Powell, T. J., Jenkins, C. D., and MacPherson, G. G. (2000). A discrete subpopulation of dendritic cells transports apoptotic intestinal epithelial cells to T cell areas of mesenteric lymph nodes, J Exp Med 191, 435-444.
Jonuleit, H., Schmitt, E., Schuler, G., Knop, J., and Enk, A. H. (2000). Induction of interleukin 10-producing, non-proliferating CD4⁺ T cells with regulatory properties by repetitive stimulation with allogeneic immature human dendritic cells, J Exp Med 192, 1213-1222.
Kaisho, T. & Akira, S. (2002). Toll-like receptors as adjuvant receptors. Biochim Biophys Acta 1589, 1-13.
Kamath, A. T., Pooley, J., O'Keeffe, M. A., Vremec, D., Zhan, Y., Lew, A. M., D'Amico, A., Wu, L., Tough, D. F. & Shortman, K. (2000). The development, maturation, and turnover rate of mouse spleen dendritic cell populations. J Immunol 165, 6762-6770.
Kawabe, T., Naka, T., Yoshida, K., Tanaka, T., Fujiwara, H., Suematsu, S., Yoshida, N., Kishimoto, T., and Kikutani, H. (1994). The immune responses in CD40-deficient mice: impaired immunoglobulin class switching and germinal center formation, Immunity 1, 167-178.
Kirk, A. D. (2001). Hitting the reset button for immune tolerance, Nat Med 1, 24-25.
Koski, G. K., Lyakh, L. A., Cohen, P. A. & Rice, N. R. (2001). CD14+ monocytes as dendritic cell precursors: diverse maturation-inducing pathways lead to common activation of NF-kappab/RelB. Crit Rev Immunol 21, 179-189.
Kumanogoh, A., Wang, X., Lee, I., Watanabe, C., Kamanaka, M., Shi, W., Yoshida, K., Sato, T., Habu, S., Itoh, M., et al. (2001). Increased T cell autoreactivity in the absence of CD40-CD40 ligand interactions: a role of CD40 in regulatory T cell development, J Immunol 166, 353-360.
Lawlor, K. E., Campbell, I. K., O'Donnell, K., Wu, L., and Wicks, I. P. (2001). Molecular and cellular mediators of interleukin-1-dependent acute inflammatory arthritis, Arthritis Rheum 44, 442-450.
Lee, J. I., Ganster, R. W., Geller, D. A., Burckart, G. J., Thomson, A. W., and Lu, L. (1999). Cyclosporine A inhibits the expression of costimulatory molecules on in vitro-generated dendritic cells: association with reduced nuclear translocation of nuclear factor kappa B, Transplantation 68, 1255-1263.
Loudovaris, M., Hansen, M., Suen, Y., Lee, S. M., Casing, P. & Bender, J. G. (2001). Differential effects of autologous serum on CD34(+) or monocyte-derived dendritic cells. J Hematother Stem Cell Res 10, 569-578.
Lu L, Woo J, Rao A S, Li Y, Watkins S C, Qian S, Starzl T E, Demetris A J, and Thomson A W. (1994). Propagation of dendritic cell progenitors from normal mouse liver using granulocyte/macrophage colony-stimulating factor and their maturational development in the presence of type-1 collagen. J Exp Med 179, 1823-1834.
Lu, L., Li, W., Fu, F., Chambers, F. G., Qian, S., Fung, J. J., and Thomson, A. W. (1997). Blockade of the CD40-CD40 ligand pathway potentiates the capacity of donor-derived dendritic cell progenitors to induce long-term cardiac allograft survival, Transplantation 64, 1808-1815.
Lutz, M. B., Suri, R. M., Niimi, M., Ogilvie, A. L., Kukutsch, N. A., Rossner, S., Schuler, G., and Austyn, J. M. (2000). Immature dendritic cells generated with low doses of GM-CSF in the absence of IL-4 are maturation resistant and prolong allograft survival in vivo, Eur J Immunol 30, 1813-1822.
MacDonald, A. S., Patton, E. A., La Flamme, A. C., Araujo, M. I., Huxtable, C. R., Bauman, B., and Pearce, E. J. (2002a). Impaired Th2 development and increased mortality during Schistosoma mansoni infection in the absence of CD40/CD 154 interaction, J Immunol 168, 4643-4649.
MacDonald, A. S., Straw, A. D., Dalton, N. M., and Pearce, E. J. (2002b). Cutting edge: Th2 response induction by dendritic cells: a role for CD40, J Immunol 168, 537-540.
MacDonald, K. P. A., Nishioka, N., Lipsky, P. E., and Thomas, R. (1997). Functional CD40-ligand is expressed by T cells in rheumatoid arthritis., J Clin Invest 100, 2404-2414.
Mackensen, A., Herbst, B., Chen, J. L., Kohler, G., Noppen, C., Herr, W., Spagnoli, G. C., Cerundolo, V. & Lindemann, A. (2000). Phase I study in melanoma patients of a vaccine with peptide-pulsed dendritic cells generated in vitro from CD34(+) hematopoietic progenitor cells. Int J Cancer 86, 385-392.
Maloy, K. J., and Powrie, F. (2001). Regulatory T cells in the control of immune pathology, Nat Immunol 2, S 16-822.
Marriott, I., Thomas, E. K., and Bost, K. L. (1999). CD40-CD40 ligand interactions augment survival of normal mice, but not CD40 ligand knockout mice, challenged orally with Salmonella dublin, Infect Immun 67, 5253-5257.
McIlroy D, Troadec C, Grassi F, Samri A, Barrou B, Autran B, Debre P, Feuillard and J, Hosmalin A. (2001). Investigation of human spleen dendritic cell phenotype and distribution reveals evidence of in vivo activation in a subset of organ donors. Blood 97, 3470-3477
Mehling, A., Grabbe, S., Voskort, M., Schwarz, T., Luger, T. A., and Beissert, S. (2000). Mycophenolate mofetil impairs the maturation and function of murine dendritic cells, J Immunol 165, 2374-2381.
Mellman, I., and Steinman, R. M. (2001). Dendritic cells: specialised and regulated antigen processing machines, Cell 106, 255-258.
Metcalf, D. & Burgess, A. W. (1982). Clonal analysis of progenitor cell commitment of granulocyte or macrophage production. J Cell Physiol, 111, 275-283. Nakajima, A., Seroogy, C. M., Sandora, M. R., Tamer, 1. H., Costa, G. L., Taylor-Edwards, C., Bachmann, M. H., Contag, C. H., and Fathman, C. G. (2001). Antigen-specific T cell-mediated gene therapy in collagen-induced arthritis, J Clin Invest 107, 1293-1301.
Nestle, F. O., Alijagic, S., Gilliet, M., Sun, Y., Grabbe, S., Dummer, R., Burg, G. & Schadendorf, D. (1998). Vaccination of melanoma patients with peptide- or tumor lysate-pulsed dendritic cells. Nat Med 4, 328-332.
Neumann, M., Fries, H., Scheicher, C., Keikavoussi, P., Kolb-Maurer, A., Brocker, E., Serfling, E., and Kampgen, E. (2000). Differential expression of Rel/NF-kappaB and octamer factors is a hallmark of the generation and maturation of dendritic cells, Blood 95, 277-285.
O'Doherty, U., Peng, M., Gezelter, S., Swiggard, W. J., Betjes, M., Bhardwaj, N. & Steinman, R. M. (1994). Human blood contains two subsets of dendritic cells, one immunologically mature and the other immature. Immunology 82, 487-493.
O'Doherty, U., Steinman, R. M., Peng, M., Cameron, P. U., Gezelter, S., Kopeloff, I., Swiggard, W. J., Pope, M. & Bhardwaj, N. (1993). Dendritic cells freshly isolated from human blood express CD4 and mature into typical immunostimulatory dendritic cells after culture in monocyte-conditioned medium. J Exp Med 178, 1.067-1078.
O'Sullivan, B. J., MacDonald, K: P., Pettit, A. R., and Thomas, R. (2000). RelB nuclear translocation regulates B cell MHC molecule, CD40 expression, and antigen-presenting cell function, Proc Natl Acad Sci USA 97, 11421-11426.
O'Sullivan, B. J., and Thomas, R. (2002). CD40 Ligation conditions dendritic cell antigen-presenting function through sustained activation of NF-kappaB, J Immunol 168, 5491-5498.
Ouaaz, F., Arron, J., Zheng, Y., Choi, Y., and Beg, A. A. (2002). Dendritic cell development and survival require distinct T-kappaB subunits, Immunity 16, 257-270.
Ozaki, M. E., Coren, B. A., Huynh, T. N., Redondo, D. J., Kikutani, H., and Webb, S. R. (1999). CD4+ T cell responses to CD40-deficient APCs: defects in proliferation and negative selection apply only with B cells as APCs, J Immunol 163, 5250-5256.
Padigel, U. M., Perrin, P. J., and Farrell, J. P. (2001). The development of a Th1-type response and resistance to Leishmania major infection in the absence of CD40-CD40L costimulation, J Immunol 167, 5874-5879.
Pai, S., O'Sullivan, B. J., Cooper, L., Thomas, R., and Khanna, R. (2002). RelB nuclear translocation mediated by C-terminal activator regions of Epstein-Barr virus-encoded latent membrane protein 1 and its effect on antigen-presenting function in B cells, J Virol 76, 1914-1921.
Pavli P, Woodhams C E, Doe W F, Hume D A. (1990). Isolation and characterization of antigen-presenting dendritic cells from the mouse intestinal lamina propria. Immunology 70(1), 40-47
Peguet-Navarro, J., Dalbiez-Gauthier, C., Rattis, F. M., Van Kooten, C., Banchereau, J. & Schmitt, D. (1995). Functional expression of CD40 antigen on human epidermal Langerhans cells. J Immunol 1-55, 4241-4247.
Pettit, A. R., Cavanagh, L. & Thomas, R. (1998). Identification and isolation of synovial dendritic cells. Meth Molec Med In Press.
Pettit, A. R., MacDonald, K. P. A., O'Sullivan, B., and Thomas, R. (2000). Differentiated dendritic cells expressing nuclear RelB are predominantly located in rheumatoid synovial tissue perivascular mononuclear cell aggregates, Arthritis Rheum 43, 791-800.
Pettit, A. R., Quinn, C., MacDonald, K. P., Cavanagh, L. L., Thomas, G., Townsend, W., Handel, M., and Thomas, R. (1997). Nuclear localization of RelB is associated with effective antigen-presenting cell function, J Immunol 159, 3681-3691.
Pierce, J. W., Schoenleber, R., Jesmok, G., Best, J., Moore, S. A., Collins, T., and Gerritsen, M. E. (1997). Novel inhibitors of cytokine-induced IkappaBalpha phosphorylation and endothelial cell adhesion molecule expression show anti-inflammatory effects in vivo, J Biol Chem 272, 21096-21103.
Pulendran, B., Banchereau, J., Burkeholder, S., Kraus, E., Guinet, E., Chalouni, C., Caron, D., Maliszewski, C., Davoust, J., Fay, J. & Palucka, K. (2000). Flt3-ligand and granulocyte colony-stimulating factor mobilize distinct human dendritic cell subsets in vivo. J Immunol 165, 566-572.
Rawson P, Hermans I F, Huck S P, Roberts J M, Pircher H, Ronchese F. (2000). Immunotherapy with dendritic cells and tumor major histocompatibility complex class I-derived peptides requires a high density of antigen on tumor cells. Cancer Res 60(16), 4493-4498.
Rescigno, M., Martino, M., Sutherland, C. L., Gold, M. R., and Ricciardi-Castagnoli, P. (1998). Dendritic cell survival and maturation are regulated by different signaling pathways, J Exp Med 188, 2175-2180.
Ridge, J. P., Di Rosa, F., and Matzinger, P. (1998). A conditioned dendritic cell can be a temporal bridge between a CD4⁺ T-helper and T-killer cell, Nature 393, 474-478.
Rissoan, M. C., Soumelis, V., Kadowaki, N., Grouard, G., Briere, F., de Waal Malefyt, R. & Liu, Y. J. (1999). Reciprocal control of T helper cell and dendritic cell differentiation. Science 283, 1183-1186.
Roncarolo. M.-G., and Levings, M. K. (2000). The role of different subsets of T regulatory cells in controlling autoimmunity, Curr Opin Immunol 12, 676-683.
Robinson, S., Mosley, R. L., Parajuli, P., Pisarev, V., Sublet, J., Ulrich, A. & Talmadge, J. (2000). Comparison of the hematopoietic activity of flt-3 ligand and granulocyte-macrophage colony-stimulating factor acting alone or in combination. J Hematother Stem Cell Res 9, 711-720.
Romani, N., Gruner, S., Brang, D., Kampgen, E., Lenz, A., Trockenbacher, B., Konwalinka, G., Fritsch, P. O., Steinman, R. M. & Schuler, G. (1994). Proliferating dendritic cell progenitors in human blood. J Exp Med 180, 83-93.
Romani, N., Reider, D., Heuer, M., Ebner, O., Kampgen, E., Eibl, B., Niederwieser, D. & Schuler, G. (1996). Generation of mature dendritic cells from human blood. An improved method with special regard to clinical applicability. J Immunol Meth 196, 137-151.
Roncarolo, M.-G., Levings, M. K., and Traversari, C. (2001). Differentiation of T regulatory cells by immature dendritic cells, J Exp Med 193, F5-F9.
Sakata, A., Sakata, K., Ping, H., Ohmura, T., Tsukano, M., and Kakimoto, K. (1996). Successful induction of severe destructive arthritis by the transfer of in vitro-activated synovial fluid T cells from patients with rheumatoid arthritis (RA) in severe combined immunodeficient (SCID) mice, Clin Exp Immunol 104, 247-254.
Sander, B., Andersson, J., and Andersson, U. (1991). Assessment of cytokines by immunofluorescence and the paraformaldehyde-saponin procedure, Immunol Rev 119, 65-93.
Schulz, O., Edwards, D. A., Schito, M., Aliberti, J., Manickasingham, S., Sher, A., and Reis e Sousa, C. (2000). CD40 triggering of heterodimeric IL-12 p70 production by dendritic cells in vivo requires a microbial priming signal, Immunity 13, 453-462.
Sha, W. C., Liou, H. C., Tuomanen, E. I., and Baltimore, D. (1995). Targeted disruption of the p50 subunit of NF-kappa B leads to multifocal defects in immune responses, Cell 80, 321-330.
Sorg, R. V., Andres, S., Kogler, G., Fischer, J. & Wernet, P. (2001). Phenotypic and functional comparison of monocytes from cord blood and granulocyte colony-stimulating factor-mobilized apheresis products. Exp Hematol 29, 1289-1294.
Steinbrink, K., Wolfl, M., Jonuleit, H., Knop, J., and Enk, A. (1997). Induction of tolerance by IL-10-treated dendritic cells, J Immunol 159, 4772-4780.
Strobl, H., Bello-Fernandez, C., Riedl, E., Pickl, W. F., Majdic, O., Lyman, S. D. & Knapp, W. (1997a). flt3 ligand in cooperation with transforming growth factor-β1 potentiates in vitro development of Langerhans-type dendritic cells and allows single-cell dendritic cell cluster formation under serum-free conditions. Blood 90, 1425-1434.
Strobl, H., Riedl, E., Scheinecker, C., Bello-Fernandez, C., Pickl, W. F., Majdic, O. & Knapp, W. (1997b). TGF-beta 1 dependent generation of LAG+dendritic cells from CD34+progenitors in serum-free medium. Adv Exp Med Biol 417, 161-165.
Strobl, H., Riedl, E., Scheinecker, C., Bello-Fernandez, C., Pickl, W. F., Rappersberger, K., Majdic, O. & Knapp, W. (1996). TGF-beta 1 promotes in vitro development of dendritic cells from CD34+ hemopoietic progenitors. J Immunol 157, 1499-1507.
Subauste, C. S., Wessendarp, M., Sorensen, R. U., and Leiva, L. E. (1999). CD40-CD40 ligand interaction is central to cell-mediated immunity against Toxoplasma gondii: patients with hyper IgM syndrome have a defective type I immune response that can be restored by soluble CD40 ligand trimer, J Immunol 162, 6690-6700.
Thomas, R., Davis, L. S. & Lipsky, P. E. (1993a). Isolation and characterization of human peripheral blood dendritic cells. J Immunol 150, 821-834.
Thomas, R., Davis, L. S. & Lipsky, P. E. (1993b). Comparative accessory cell function of human peripheral blood dendritic cells and monocytes. J Immunol 151(12), 6840-6852.
Thomas, R., Davis, L. S. & Lipsky, P. E. (1994a). Rheumatoid synovium is enriched in mature antigen-presenting dendritic cells. J Immunol 152, 2613-2623.
Thomas, R. & Lipsky, P. E. (1994). Human peripheral blood dendritic cell subsets: Isolation and characterization of precursor and mature antigen-presenting cells. J Immunol 153, 4016-4028.
Thomas, R., McIlraith, M., Davis, L. S. & Lipsky, P. E. (1994b). Rheumatoid synovial T cells contribute to the pathogenesis of R-A: comment on the article by Matthews et al. (letter). Arthritis Rheum 37(4).
Thompson, A. G., Pettit, A. R., Padmanabha, J., Mansfield, H., Frazer, I. H., Strutton, G. M., and Thomas, R. (2002). Nuclear RelB+ cells are found in normal lymphoid organs and in peripheral tissue in the context of inflammation, but not under normal resting conditions. Immunol Cell Biol 80, 164-169.
Thurner, B., Roder, C., Dieckmann, D., Heuer, M., Kruse, M., Glaser, A., Keikavoussi, P., Kampgen, E., Bender, A. & Schuler, G. (1999). Generation of large numbers of full), mature and stable dendritic cells from leukapheresis products for clinical application. J Immunol Methods 223, 1-15.
van den Berg W B, van Beusekom H J, van de Putte L B, Zwarts W A, van der Sluis M. (1982). Antigen handling in antigen-induced arthritis in mice: an autoradiographic and immunofluorescence study using whole joint sections. An J Pathol 108, 9-16.
Vremec, D. & Shortman, K. (1997). Dendritic cell subtypes in mouse lymphoid organs. Cross-correlation of surface markers, changes with incubation, and differences among thymus, spleen, and lymph nodes. J Immunol 159, 565-573.
Vremec D, Pooley J, Hochrein H, Wu L, and Shortman K. (2000). CD4 and CD8 expression by dendritic cell subtypes in mouse thymus and spleen. J Immunol 164, 2978-2986.
Vuckovic, S., Fearnley, D. B., Mannering, S. I., Dekker, J., Whyte, L. F. & Hart, D. N. (1998). Generation of CMRF-44+ monocyte-derived dendritic cells: insights into phenotype and function. Exp Hematol 26, 1255-1264.
Weih, F., Carrasco, D., Durham, S. K., Barton, D. S., Rizzo, C. A., Ryseck, R. P., Lira, S. A., and Bravo, R. (1995). Multiorgan inflammation and hematopoietic abnormalities in mice with a targeted disruption of RelB, a member of the NF-kappa B/Rel family, Cell 80, 331-340.
Weih, F., Warr, G., Yang, H., and Bravo, R. (1997). Multifocal defects in immune responses in RelB-deficient mice, J Immunol 158, 5211-5218.
Wu, L., A., D. A., Winkel, K. D., Suter, M., Lo, D., and Shortman, K. (1998). RelB is essential for the development of myeloid-related CD8alpha− dendritic cells but not of lymphoid-related CD8alpha+ dendritic cells, Immunity 9, 839-847.
Yoshimura, S., Bondeson, J., Foxwell, B. M., Brennan, F. M., and Feldmann, M. (2001). Effective antigen presentation by dendritic cells in NF-kappaB dependent coordinate regulation of MHC, costimulatory molecules and cytokines, Int Immunol 13, 675-683.
Zheng, Z., Takahashi, M., Narita, M., Toba, K., Liu, A., Furukawa, T., Koike, T. & Aizawa, Y. (2000). Generation of dendritic cells from adherent cells of cord blood by culture with granulocyte-macrophage colony-stimulating factor, interleukin-4, and tumor necrosis factor-alpha. J Hematother Stem Cell Res 9, 453-464.
Zhou, P., and Seder, R. A. (1998). CD40 ligand is not essential for induction of type 1 cytokine responses or protective immunity after primary or secondary infection with histoplasma capsulatum, J Exp Med 187, 1315-1324.

1. An isolated antigen-presenting cell for modulating an immune response, which is characterised by producing CD40, or its equivalent, at a level or functional activity that is less than about 50% of that produced by an activated dendritic cell.

2. An antigen-presenting cell according to claim 1, wherein CD40, or its equivalent, is produced at a level or functional activity that is less than about 1% of that produced by an activated dendritic cell.

3. An antigen-presenting cell according to claim 1, which cannot be induced to express CD40, or its equivalent, at an equivalent level and/or functional activity as that produced by an activated antigen presenting cell.

4. An antigen-presenting cell according to claim 1, wherein CD40, or its equivalent, is produced at a level or functional activity that is lower than that produced by an immature dendritic cell.

5. An antigen-presenting cell according to claim 1, which cannot be induced to express CD40, or its equivalent, at a higher level and/or functional activity than that produced by an immature antigen-presenting cell.

6. An antigen-presenting cell according to claim 1, which is other than a B lymphocyte.

7. An antigen-presenting cell according to claim 1, which is selected from monocytes, macrophages, cells of myeloid lineage, dendritic cells or Langerhans cells.

8. An antigen-presenting cell according to claim 1, which is a dendritic cell.

9. An antigen-presenting cell according to claim 1, which is a macrophage.

10. An antigen-presenting cell according to claim 1, which produces NF-κB or a component thereof, at a level or functional activity which is lower than that produced by a mature or activated dendritic cell.

11. An antigen-presenting cell according to claim 1, which cannot be induced to express NF-κB or component thereof, at a higher level and/or functional activity than an immature antigen presenting cell.

12. An antigen-presenting cell according to claim 1, which produces NF-κB or a component thereof, at a level or functional activity that is lower than that produced by an immature dendritic cell.

13. An antigen-presenting cell according to any one of claims 10 to 12, wherein the component is RelB.

14. An antigen-presenting cell according to claim 1, which produces an immunostimulatory molecule.

15. An antigen-presenting cell according to claim 14, wherein the immunostimulatory molecule comprises CD86 or its equivalent.

16. An antigen-presenting cell according to claim 14, wherein the immunostimulatory molecule is produced at a level or functional activity which is at least about 10% of that produced by an activated dendritic cell.

17. An antigen-presenting cell according to claim 14, wherein the immunostimulatory molecule is produced at a level or functional activity which is the same as that produced by an activated dendritic cell.

18. An antigen-presenting cell according to claim 1, which is produced by a process comprising contacting a precursor of the antigen-presenting cell with an NF-κB inhibitor for a time and under conditions sufficient to differentiate an antigen-presenting cell from the precursor and to inhibit or otherwise reduce the level and/or functional activity of NF-κB in the cell.

19. An antigen-presenting cell according to claim 18, wherein the precursor is derived from monocytes or bone marrow.

20. An antigen-presenting cell according to claim 18, wherein the NF-κB inhibitor inhibits nuclear translocation of NF-κB, or a component thereof.

21. An antigen-presenting cell according to claim 18, wherein the NF-κB inhibitor inhibits nuclear translocation of RelB.

22. An antigen-presenting cell according to claim 18, wherein the NF-κB inhibitor is an antisense nucleic acid molecule or oligonuclectide, which is complementary or encodes at least a portion of a NF-κB subunit selected from p50, p65 or RelB.

23. An antigen-presenting cell according to claim 18, wherein the NF-κB inhibitor is an inhibitor of RelB or p50.

24. An antigen-presenting cell according to claim 18, wherein the NF-κB inhibitor is an inhibitor of RelB or p50, which is selected from a ribozyme that selectively destroys RNA encoding NF-κB or component thereof, an antisense molecule which prevents transcription of NF-κB or component thereof, or an antigen-binding molecule that blocks NF-κB action.

25. An antigen-presenting cell according to claim 18, wherein the NF-κB inhibitor is an indirect inhibitor of NF-κB selected from inhibitors of IκB degradation, inhibitors of IκB phosphorylation, inhibitors of IκB ubiquitination and inhibitors of proteolytic degradation of IκB.

26. An antigen-presenting cell according to claim 18, wherein the NF-κB inhibitor is an inhibitor of IκB phosphorylation.

27. An antigen-presenting cell according to claim 18, wherein the NF-κB inhibitor is BAY 11-7082.

28. An antigen-presenting cell according to claim 18, wherein the NF-κB inhibitor is an indirect inhibitor of NF-κB selected from inhibitors of proteolysis and inhibitors of nuclear translocation of NF-κB.

29. An antigen-presenting cell according to claim 18, wherein the NF-κB inhibitor is an inhibitor of nuclear translocation of NF-κB selected from deoxyspergualin or deoxyspergualin derivatives or analogues.

30. An antigen-presenting cell according to claim 18, wherein the NF-κB inhibitor is an inhibitor of proteolysis selected from proteosome inhibitors.

31. An antigen-presenting cell according to claim 18, wherein the NF-κB inhibitor is a proteosome inhibitor selected from PSI, ALLN, lactacystin, MG-132, C-LFF and calpain inhibitors.

32. An antigen-presenting cell according to claim 18, wherein the NF-κB inhibitor is an indirect inhibitor of NF-κB selected from caffeic acid phenethyl ester, pyrrolidine dithiocarbonate, lovastatin, aselastine HCL, tepaxalin, (−)-epi gallocatechin-3-gallate, phenyl-N-tert-butylnitrone, quercetin, cucumin or E330.

33. An antigen-presenting cell according to claim 18, wherein the NF-κB inhibitor inhibits proteolysis.

34. An antigen-presenting cell according to claim 18, wherein the NF-κB inhibitor is a proteosome inhibitor.

35. An antigen-presenting cell according to claim 1, which is produced by a process comprising contacting an antigen-presenting cell, or its precursor, with an inhibitor of CD40, or its equivalent, for a time and under conditions sufficient to produce a modified antigen-presenting cell that produces CD40, or its equivalent, at a reduced or abrogated level or functional activity relative to that of the antigen-presenting cell or its precursor.

36. An antigen-presenting cell according to claim 35, wherein the process further comprises contacting the antigen-presenting cell, or its precursor, or the modified antigen-presenting cell, with an agent that increases the level or functional activity of an immunostimulatory molecule for a time and under conditions sufficient to enhance or otherwise elevate the level or functional activity of the immunostimulatory molecule in the antigen-presenting cell, or its precursor, or the modified antigen-presenting cell.

37. An antigen-presenting cell according to claim 35, wherein the process further comprises contacting the antigen-presenting cell, or its precursor, or the modified antigen-presenting cell, with an agent that increases the level or functional activity of CD86 or its equivalent, for a time and under conditions sufficient to enhance or otherwise elevate the level or functional activity of CD86 or its equivalent in the antigen-presenting cell, or its precursor, or the modified antigen-presenting cell.

38. A method of producing antigen-presenting cells for modulating an immune response to a target antigen, comprising contacting an antigen-presenting cell, or its precursor, with an antigen that corresponds to the target antigen, or with a polynucleotide from which the antigen is expressible, for a time and under conditions sufficient for the antigen or a processed form thereof to be presented by the antigen-presenting cell, or its precursor, wherein antigen-presenting cell, or its precursor, is characterized by producing CD40, or its equivalent, at a level or functional activity that is less than about 50% of that produced by an activated dendritic cell.

39. A method according to claim 38, wherein the antigen presentation is restricted by major histocompatability (MHC) molecules.

40. An antigen-specific antigen-presenting cell for modulating an immune response to a target antigen, which is produced by contacting an antigen-presenting cell, or its precursor, with an antigen that corresponds to the target antigen, or with a polynucleotide from which the antigen is expressible, for a time and under conditions sufficient for the antigen or a processed form thereof to be presented by the antigen-presenting cell, or its precursor, wherein antigen-presenting cell, or its precursor, is characterised by producing CD40, or its equivalent, at a level or functional activity that is less than about 50% of that produced by an activated dendritic cell.

41. A method of producing antigen-presenting cells for modulating an immune response to a target antigen, comprising contacting a precursor of the antigen-presenting cell with an NF-κB inhibitor and with an antigen that corresponds to the target antigen, or with a polynucleotide from which the antigen is expressible, for a time and under conditions sufficient to differentiate an antigen-presenting cell from the precursor and to inhibit or otherwise reduce the level or functional activity of NF-κB in the cell, wherein the antigen or a processed form thereof is presented by the antigen-presenting cell so produced.

42. A method according to claim 41, wherein the immune response is mediated by T lymphocytes.

43. A method according to claim 41, wherein the T lymphocytes are selected from cytotoxic T lymphocytes (CTLs) and T helper lymphocytes.

44. A method according to claim 41, wherein the antigen is selected from a protein antigen, a particulate antigen, an alloantigen, an autoantigen, an allergen, a bacterial antigen, a viral antigen or a parasitic antigen or immune complex.

45. A method according to claim 41, wherein the modulation of the immune response is selected from inducing a tolerogenic response, or the suppression of a future or existing immune response, to a specified antigen or group of antigens.

46. A method for producing T lymphocytes that exhibit anergy for a target antigen, comprising contacting a population of T lymphocytes, or their precursors, with an antigen-specific antigen-presenting cell, which is characterised by producing CD40, or its equivalent, at a level or functional activity that is less than about 50% of that produced by an activated dendritic cell, for a time and under conditions sufficient to produce the anergic T lymphocytes.

47. A method according to claim 46, wherein the T lymphocytes are selected from cytotoxic T lymphocytes (CTLs) and T helper lymphocytes.

48. A method according to claim 46, wherein the antigen is selected from a protein antigen, a particulate antigen, an alloantigen, an autoantigen, an allergen, a bacterial antigen, a viral antigen or a parasitic antigen or immune complex.

49. A T lymphocyte that exhibits anergy for a target antigen, which is produced by contacting a T lymphocyte, or its precursor, with an antigen-specific antigen-presenting cell, which is characterised by producing CD40, or its equivalent, at a level or functional activity that is less than about 50% of that produced by an activated dendritic cell, for a time and under conditions sufficient to produce the anergic T lymphocyte.

50. A method for modulating the immune response to an antigen, comprising administering to a patient in need of such treatment an antigen-specific antigen-presenting cell for a time and under conditions sufficient to modulate the immune response, wherein the antigen-specific antigen-presenting cell is produced by contacting an antigen-presenting cell with an antigen that corresponds to the target antigen, or with a polynucleotide from which the antigen is expressible, for a time and under conditions sufficient for the antigen or a processed form thereof to be presented by the antigen-presenting cell, wherein antigen-presenting cell is characterised by producing CD40, or its equivalent, at a level or functional activity that is less than about 50% of that produced by an activated dendritic cell.

51. A method for modulating the immune response to an antigen, comprising administering to a patient in need of such treatment an anergic T lymphocyte for a time and under conditions sufficient to modulate the immune response, wherein the anergic T lymphocyte is produced by contacting a population of T lymphocytes, or their precursors, with an antigen-specific antigen-presenting cell, which is characterised by producing CD40, or its equivalent, at a level or functional activity that is less than about 50% of that produced by an activated dendritic cell, for a time and under conditions sufficient to produce the anergic T lymphocytes.

52-55. (canceled)

56. A method for treatment and/or prophylaxis of a disease or condition whose symptoms or aetiology are associated with the presence of an immune response, comprising administering to a patient in need of such treatment or prophylaxis an effective amount of one or both of an antigen-specific antigen-presenting cell according to claim 40 and/or of an anergic T lymphocyte according to claim 49.

57-58. (canceled)

59. A method of treating an allergy in a subject, comprising administering to said subject a cell according to claim 40.

60. A method of treating an allergy in a subject, comprising administering to said subject a cell according to claim 49.

61. A method of treating or preventing an autoimmune disease in a subject, comprising administering to said subject a cell according to claim 40.

62. A method of treating or preventing an autoimmune disease in a subject, comprising administering to said subject a cell according to claim 49.

63. A method of treating or preventing transplant rejection disease in a subject, comprising administering to said subject a cell according to claim 40.

64. A method of treating or preventing an autoimmune disease in a subject, comprising administering to said subject a cell according to claim 49.
